SEQUENCE LISTING (15 sequences)

SEQ ID NO: 1 - protein, 609 aa, Homo sapiens [sequence omitted]

SEQ ID NO: 2 - protein, 205 aa, Homo sapiens [sequence omitted]

SEQ ID NO: 3 - protein, 410 aa, Artificial Sequence
  LAMP2B
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 4 - protein, 478 aa, Artificial Sequence
  LIMP2
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 5 - protein, 455 aa, Artificial Sequence
  TNFR
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 6 - protein, 760 aa, Artificial Sequence
  TfR1
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 7 - protein, 433 aa, Artificial Sequence
  Control Construct hCD63-Nluc
MTGAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGASGGSGSGSFEANYTDWEKIPSMSKNRVPDSCCINVTVGCGINFNEKAIHKEGCVEKIGGWLRKNVLVVAAAALGIAFVEVLGIVFACCLVKSIRSGYEVMACGSSGGSGGGSGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - DNA, 1827 bp, Homo sapiens
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacttgtgttgctgatgagtcagctgaaaattgtgacaaatcacttcatacccttttcggtgacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccaggagacatccatatttctatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtgttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggatccaaatgttgtaaacatcctgaagcaaaaagaatgccctgtgcagaggactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 9 - DNA, 615 bp, Homo sapiens
gtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtggggtccaaatgttgtaaacatcctgaagcaaaaagaatgccctgtgcagaggactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 10 - DNA, 1230 bp, Artificial Sequence
  Lamp2B
atggtttgcttcaggttgtttcctgttccaggtagcggcttggttctcgtctgtcttgtactcggcgccgtaaggagctacgctttggagctgaatcttactgactctgaaaacgccacatgtttgtatgccaagtggcagatgaatttcaccgtcaggtacgagacgactaacaagacctacaagacggtcactatcagcgaccacggcactgtgacatataacgggtccatctgcggggacgatcagaacggcccaaagattgctgttcaatttggccccggcttcagttggattgccaactttaccaaagctgcgagcacttactctattgattcagtcagtttctcatacaacaccggcgataacacgacatttccggacgcagaagataaggggattctgactgttgatgagctgctcgctattcgcatcccccttaatgacctttttcggtgcaacagtctttccacactcgagaagaatgacgtcgttcagcactactgggacgttctggtccaagcatttgtgcaaaatggcacggtaagcactaacgagtttctgtgcgacaaggacaagactagcactgtcgccccgactatccatacaactgtcccaagccctacgaccactccgactcccaaggagaaaccagaggctgggacgtatagcgtcaacaatgggaatgatacatgcttgctggccacaatgggactccaactgaatatcacccaggacaaggtggcctcagttataaatataaatcctaatacaacacatagtactggaagttgtcgctcccataccgccttgttgaggttgaacagctcaacgataaagtatctcgatttcgtctttgccgtaaagaatgaaaaccgattctatttgaaggaggtcaatataagtatgtacctggtaaacggatcagtatttagtattgctaataacaatctctcttactgggacgctcctctcggaagtagttatatgtgtaacaaggaacagaccgtctctgtatccggagcgttccagatcaatactttcgacctgcgcgtacaacctttcaatgttacgcaagggaagtactccacagcccaggagtgttctcttgacgatgacaccattttgatcccgatcattgttggagcagggttgagtgggctgattattgttattgtaatcgcttatgttataggacgaaggaagagttacgccggctatcagacgctt

SEQ ID NO: 11 - DNA, 1434 bp, Artificial Sequence
  LIMP2
atggggcgatgttgcttttacacggcgggtaccctgagcttgttgctcctcgtgacaagtgtcactcttttggtagctagggtgtttcagaaggccgtagaccaatctatagaaaaaaagattgttcttagaaatggtaccgaagcgttcgattcttgggagaaaccaccactcccggtatatacccagttctacttcttcaatgtaaccaacccagaggagattcttcgaggtgagacccctcgcgtggaagaagtaggaccatacacctatcgggaacttcgcaacaaagctaatattcaattcggggataatggcaccacgataagcgcagtaagcaataaggcgtatgtgtttgaacgagatcagagtgtgggtgatcctaagatcgacctcatccgcactctcaatatccccgtattgacagttattgagtggtctcaagttcattttctccgagagattatagaagcgatgcttaaggcgtaccaacaaaagttgttcgtcacccatactgtggatgaactcctgtggggatataaggatgaaattttgagtttgatccacgtatttagaccagacatttcaccctattttggcttgttctatgagaaaaatggaacgaacgacggggattacgtattcctcaccggtgaggactcatacctgaacttcactaaaatagtcgagtggaacggaaagacatctctggactggtggattactgataagtgcaatatgataaacggcactgacggggattctttccatcccctgatcacgaaagatgaggtactctacgtattcccgagcgatttctgtcggagcgtctacatcacattttccgattacgagagcgtccaaggtctgccagccttccggtataaagtaccagctgaaatacttgctaacaccagtgacaatgccggattttgtataccagagggtaattgtttggggagtggagtgctcaacgtgtcaatttgcaagaacggggcacctataatcatgtcttttccacacttctatcaagctgatgagcggttcgtaagcgccatagaaggtatgcatccgaatcaagaggaccatgaaaccttcgtggatataaacccgctcactggcataattttgaaggccgcaaagagattccagattaatatatacgttaagaagcttgatgatttcgttgaaaccggagacattcggactatggtatttcctgtaatgtacttgaacgaatccgtacacatcgataaggagacagcgagtcgccttaaaagtatgataaatacgactcttataatcacaaacatcccttatattataatggccttgggtgtgttctttggattggtgtttacatggttggcatgtaagggccaagggtccatggatgaggggacggctgatgaaagagcccctttgattcgcact

SEQ ID NO: 12 - DNA, 1365 bp, Artificial Sequence
  TNFR
atgggccttagtacggttccggatttgttgctcccactcgtcttgctggagcttcttgtcggcatatatcccagcggagtcatcggcttggtgccgcatcttggcgaccgagagaaacgggatagtgtgtgcccgcaagggaaatatatccatccacagaacaattccatttgttgtacaaagtgtcacaagggaacatatttgtacaacgactgtcccggacctggacaagacaccgactgtcgggagtgtgaatccgggtcttttactgcgagcgaaaaccacctcaggcattgtctcagttgctccaaatgccggaaagaaatggggcaggtggaaatttcatcatgcactgtggaccgcgacactgtttgcggttgccggaagaaccaatatcggcattactggagcgaaaatctctttcaatgtttcaactgttcactgtgtctcaacggtactgtacatctctcttgtcaggagaagcagaatactgtttgtacttgtcacgccgggttttttttgagggaaaatgagtgcgttagctgcagtaattgtaagaagtcactcgaatgcacaaagctgtgcttgccccagatagaaaacgtaaagggaacggaagatagtgggacgacggtgctcctccctctggtgatatttttcggactttgcctcctgagcctgctttttatagggctcatgtatcgctaccagcggtggaagtccaagctgtacagtatagtgtgtggtaagtctacacctgaaaaggagggggaactcgaaggaaccacaacgaagcctttggcaccgaacccgtcattttcacccacgccgggtttcaccccgacactgggattctcccccgtaccttcttcaacattcacatcaagcagtacatacactcctggggactgtcccaatttcgcagctcctaggcgcgaggtggcacccccctaccaaggtgctgatcctattctcgcaactgcactggcgagcgatccaataccgaaccccctgcagaaatgggaggattcagcccacaagccgcaatctctggatactgacgacccagccactctctatgccgtagttgagaacgtaccgcctctcaggtggaaagaattcgtgcgaaggttgggcctgtctgatcatgaaatagaccgactcgaactccagaatggtcgctgccttcgagaggctcagtactcaatgctcgctacctggcgcaggaggacaccacggagggaggctacccttgaactgcttgggcgcgtactgcgggacatggatctgcttggctgccttgaggatatagaggaggcactgtgcggccctgccgcactgccaccagccccgagtttgctgcgg

SEQ ID NO: 13 - DNA, 2280 bp, Artificial Sequence
  TfR1
atgatggaccaggcgcgatccgccttttcaaacttgttcggaggagagcctctgagttatacaaggttcagtctggcccgacaagtggacggagacaattcacacgtcgagatgaaacttgctgttgatgaggaagagaatgcggacaataatacgaaagcaaacgtgacaaaacccaaaagatgtagcgggtccatctgctacggaacgatcgccgtcatcgtcttttttcttatagggttcatgatcggatatctcgggtattgtaaaggcgtcgagccgaaaactgaatgcgaacgactcgctggaacagagtctcctgttagagaggagcctggggaggatttccctgcagcgcgacggctgtattgggatgatttgaagaggaaactgtctgaaaagctggattccacggacttcacgggcacgatcaaattgcttaatgagaattcctatgtcccgcgggaggctggttcacaaaaagacgagaacctggcgttgtatgtcgaaaatcaattccgcgaattcaagttgagtaaggtatggcgagatcaacacttcgtaaaaatacaagtcaaggattccgcgcaaaacagtgtaatcatcgttgataaaaatggacggcttgtgtatttggtggaaaatccgggcgggtatgtggcatatagcaaggccgcaactgtaacaggcaagcttgtacacgccaatttcggcaccaagaaagattttgaggacctttacactcccgtcaatggaagtatagtgattgtgagagcgggcaagattacctttgcggaaaaagtagctaatgcagagtccttgaacgcgataggggtcctcatctatatggatcagacgaagttcccgattgtaaacgccgagctgagcttcttcggacacgcgcacctgggaacgggggatccttacacaccgggatttccaagtttcaatcacactcagtttccgccgagtcgatccagtggactccctaacattcctgtccagacgatatcccgagcagcggcagaaaaacttttcgggaatatggaaggcgactgccctagcgactggaagactgattcaacttgtagaatggtgacctctgagagcaagaatgtcaagctgaccgtctccaatgtccttaaggagattaagattctgaatatcttcggagtcataaaggggttcgtagagccggatcactacgtcgtggttggagcacagagggacgcgtggggacccggtgctgcaaagagtggagtgggcacagcgttgcttcttaagctcgcgcagatgttctccgacatggtgcttaaagatgggtttcaaccctctagaagcattattttcgcatcttggtctgcaggcgatttcgggtcagttggggcgacagaatggcttgagggctacctcagttctctgcatctcaaggccttcacttatattaacctggacaaagctgtattgggtacgtcaaacttcaaggtgagtgcttccccactcctgtacacactgatagaaaagactatgcaaaatgtaaagcaccccgtgacgggccaattcctttaccaggactcaaattgggcctcaaaggtggaaaaactcactctcgacaacgcagcgttccccttcctggcctatagcggcatcccggcggtgtctttctgcttttgtgaagacaccgactacccctatttgggtaccacgatggatacctataaagagctgatagagaggatcccggaactcaataaagttgccagggcagcggccgaggtggcaggacagttcgtgattaaactcactcacgatgtcgaactgaatctcgattatgaacggtataactctcaactcctgtcctttgtccgcgatctgaaccaatacagagcggacattaaagaaatgggactgtcactccagtggctctactctgctcgcggtgacttctttagggcaacgtccagactgacgactgatttcggtaatgctgagaaaaccgatcgcttcgtgatgaagaaactgaatgacagggtgatgcgggtcgaatatcacttcctctcaccttatgtttctccaaaggagtcccctttccggcatgtcttttggggcagcggaagccatacgctgcccgcattgcttgaaaatctgaaactgagaaagcagaataatggagcatttaatgaaacgttgtttcgcaaccagcttgccctcgcgacgtggactatacagggtgcggcgaatgctttgagcggagatgtttgggatatagacaatgaattt

SEQ ID NO: 14 - DNA, 1302 bp, Artificial Sequence
  Control Construct hCD63-Nluc
atgaccggtgctgtggaaggcggcatgaaatgcgtgaagtttctgctgtatgttctgctcttggcattttgtgcgtgtgccgttggccttattgcagtcggtgtaggtgctcagttggtactcagtcaaacaattattcagggcgcaacacccggctctttgttgccggtggtgattatcgctgttggtgtattcctttttttggtggcctttgttggttgctgcggggcgtgcaaggagaattactgtttgatgattacctttgccatatttctgtcattgattatgctggtagaggttgcagcggcgattgcagggtacgtgtttagagataaagtgatgtcagagtttaacaacaattttaggcagcagatggaaaattacccgaaaaacaaccacacagcttcaatcctggacagaatgcaagcggactttaaatgttgcggtgcctccggaggtagcggcagcggtagcttcgaagcgaactatactgattgggagaaaattccgagtatgtctaagaatcgagtaccagatagttgctgtataaatgtgacggtcggatgcggaataaacttcaatgaaaaggctattcacaaagagggatgtgtcgagaaaattggtggatggctgaggaagaatgtactggtggttgctgccgccgccctgggcatcgcattcgtagaggtcctcggaatcgtatttgcgtgttgtttggttaagtctatacggagcgggtatgaagtcatggcatgcggcagttctggaggctcaggcggcggatctggcgtcttcacattggaagacttcgttggggattggaggcagaccgccgggtataacttggatcaggtactcgaacaaggcggagtatcttccctgttccaaaatctgggggttagcgtcacgcccattcaacgcattgtgctctccggtgagaatggactgaagatcgatatacacgtaattatcccctatgaggggctgtctggtgatcagatggggcagatagagaaaatctttaaggtagtttaccccgtggatgatcaccacttcaaagtaatcctccattatggtacactcgtgattgacggggtcacaccaaacatgatcgattacttcggcagaccgtatgagggcatagcagtattcgacggaaagaagataacagtcacggggacattgtggaatggaaacaaaataattgatgaacgcctcatcaacccggacggaagtcttctttttcgcgtcacaatcaacggcgtgacggggtggcggctctgtgaaaggatcttggcgtag

SEQ ID NO: 15 - DNA, 510 bp, Artificial Sequence
  Nluc
gtcttcacattggaagacttcgttggggattggaggcagaccgccgggtataacttggatcaggtactcgaacaaggcggagtatcttccctgttccaaaatctgggggttagcgtcacgcccattcaacgcattgtgctctccggtgagaatggactgaagatcgatatacacgtaattatcccctatgaggggctgtctggtgatcagatggggcagatagagaaaatctttaaggtagtttaccccgtggatgatcaccacttcaaagtaatcctccattatggtacactcgtgattgacggggtcacaccaaacatgatcgattacttcggcagaccgtatgagggcatagcagtattcgacggaaagaagataacagtcacggggacattgtggaatggaaacaaaataattgatgaacgcctcatcaacccggacggaagtcttctttttcgcgtcacaatcaacggcgtgacggggtggcggctctgtgaaaggatcttggcg

(19) United States
(12) Patent Application Publication
(10) Pub. No.: US 2024/0197908 A1
(43) Pub. Date: Jun. 20, 2024

(54) MODIFIED EXTRACELLULAR VESICLES (EVS) WITH IMPROVED HALF-LIFE

(71) Applicant: Evox Therapeutics Limited, Oxford (GB)

(72) Inventors: Christopher DAVIES, Oxford (GB); Justin HEAN, Oxford (GB); Xiuming LIANG, Stockholm (SE)

(21) Appl. No.: 18/287,206

(22) PCT Filed: Apr. 26, 2022
(86) PCT No.: PCT/EP2022/061104

§ 371 Date: Oct. 17, 2023

(30) Foreign Application Priority Data
Apr. 26, 2021 (GB) 2105925.8

(51) Int. Cl. A61K 47/69; A61K 47/64; C12N 5/0775; C12P 21/02

(52) U.S. Cl. CPC A61K 47/6901; A61K 47/64; A61K 47/643; C12N 5/0662; C12P 21/02

(57) ABSTRACT

A modified Extracellular Vesicle (EV), preferably an exosome, comprising an albumin protein or fragment (e.g., an albumin domain) present on the surface of the EV membrane, preferably as part of a fusion protein (e.g., a transmembrane protein). The EV exhibits an improved circulating half-life, stability and shelf-life (both formulation and storage). Methods of making said EVs, as well as pharmaceutical compositions and their use.

### TECHNICAL FIELD

The present invention relates to modified Extracellular Vesicles (EVs), preferably exosomes, having an improved pharmacokinetic profile, increased half-life and stability, methods of making said EVs, as well as compositions thereof and their use as a medicinal agent.

### BACKGROUND ART

Extracellular Vesicles (EVs), such as exosomes, have several potential therapeutic uses and EVs are already being investigated as delivery vehicles for protein, nucleic acid, small molecule therapeutics and biologics.

However, the promising potential for clinical applications of EVs in the treatment of diseases is currently impacted by the rapid clearance of EVs, especially after intravenous administration. Due to their short half-life, the majority of untargeted EVs home to the liver and spleen, meaning that the therapeutic applications of untargeted EV therapeutics are predominantly focused on these target organs.

The short circulation time of EVs in vivo is one of the major limitations to exploiting their considerable therapeutic potential. Furthermore, EVs are known to be predominantly taken up by the liver and the spleen. This distinct biodistribution pattern means that targeting organs other than the hepatosplenic system requires large doses and/or the inclusion of specific targeting moieties to direct the EVs to other target organs. It is desirable to divert EVs away from uptake by the liver and the spleen so that this will increase delivery to other organs and thus improve biodistribution (especially of targeted EVs for instance to the brain).

Several commonly employed techniques to increase the half-life of drugs and small biologics exist. Typically, one of four general strategies for prolongation of half-life are used:
1) Noncovalent binding or genetic fusion of a pharmacologically active peptide or protein to a naturally long-half-life protein or protein domain.
2) Increasing the hydrodynamic radius by chemical conjugation of the pharmacologically active peptide or protein to repeat chemical moieties, e.g., to polyethylene glycol (PEG) (PEGylation) or hyaluronic acid.
3) Significantly increasing the negative charge of the pharmacologically active peptide or protein by polysialylation; or, alternatively, fusing a negatively charged, highly sialylated peptide (e.g., carboxy-terminal peptide [CTP; of chorionic gonadotropin (CG) β-chain]), known to extend the half-life of natural proteins such as human CG β-subunit, to the biological drug candidate.
4) Coating or conjugating the pharmacological agents (e.g., lipid or polymer nanoparticle or a therapeutic protein) with PEG of different lengths and structure, to decrease the electrostatic charge of a cargo molecule and shield it from immune systems cells, renal or liver clearance mechanisms.

These current approaches, except for PEGylation, have been used with limited success clinically with recombinant proteins and in some instances RNA therapeutics, but never with a large macromolecular assembly in the form of a nanoparticle, such as an EV, which is impacted by completely different biophysical and immunological considerations in vivo. In particular, EVs are much larger than a single protein or RNA therapeutic. EVs also carry a very different charge compared to single protein or RNA therapeutics, which makes the translation of existing methods for altering pharmacokinetics/pharmacodynamics into the EV context very unpredictable.

Existing methods described above have some significant drawbacks. The main concern with noncovalent binding or genetic fusion of a pharmacologically active peptide or protein to a naturally long-half-life protein or protein domain is the stability and aberrant glycosylation in linkers and fusion proteins. Fusion proteins can also be bulky and can lead to problems with interference of the fusion protein with the activity of the therapeutic.

PEGylation of EVs has been attempted in the past with little success. Firstly, it is a challenge to conjugate PEG to EVs without disturbing the EV topology and phenotype. Secondly, PEG is an artificial substance which may cause toxic side effects when injected. It is furthermore immunogenic, which can also cause toxicity as well as clearance of the drug product from circulation.

Sialylation in the context of EVs would be unsuitable because there are receptors that recognise sialylated moieties that drive uptake for certain EV subclasses. For example, B-cell exosomes are taken up by sialoadhesin in the spleen. Increasing the glycosylation pattern on EVs is not easy because EVs comprise a membrane, similar to the plasma membrane; hence they already have glycosylated proteins on their surface. Proteins commonly found on EVs are known to be heavily glycosylated and thereby negatively charged, so there would be no benefit to increase the sialylation of the EV surface.

There remains a need in the art for EVs with improved therapeutic potential, for example, EVs with longer circulation times in vivo, requiring lower doses of EVs to be delivered and/or more targeted delivery of EVs to organs, for example, other than the hepatosplenic system, thus improving biodistribution to organs such as the brain.

### SUMMARY OF INVENTION

An object of the invention is to overcome at least one of the afore-mentioned problems associated with half-life and biodistribution of EVs.

In a first aspect the present invention relates to an Extracellular Vesicle (EV) modified to comprise an albumin protein present on the surface of the EV. The albumin protein may form part of a fusion protein with an EV protein, optionally wherein the EV protein is a transmembrane EV protein or an EV protein associated with the outer surface of the EV membrane. The EV may comprise a cargo. Optionally, the EV is an exosome.

In a second aspect the present invention relates to a method for producing an EV according to the first aspect, comprising: (i) introducing into an EV-producing cell at least one polynucleotide construct encoding an albumin-EV protein fusion construct; and (ii) expressing said construct in the EV-producing cell, thereby generating an EV comprising an albumin protein present on the surface of the EV.

In a third aspect the present invention relates to a pharmaceutical composition comprising at least one EV according to the first aspect and a pharmaceutically acceptable excipient, diluent, vehicle, solvent, or carrier.

In a fourth aspect the present invention relates to an EV according to the first aspect and/or a pharmaceutical composition according to the third aspect, for use in medicine. In another aspect, the invention relates to a method of treating a disease or condition as described herein in a subject in need thereof, comprising administering to the subject an EV according to the first aspect and/or a pharmaceutical composition according to the third aspect.

### BRIEF DESCRIPTION OF FIGURES

The invention will be more clearly understood by reference to the accompanying Figures, in which:

FIG. 1 shows a schematic illustration of albumin engineered into an EV.

FIG. 2 shows a schematic illustration of albumin constructs for engineering albumin into an EV (2A) N-terminal display, (2B) C-terminal display and (2C) intraloop display.

FIG. 3 shows a western blot demonstrating expression of fusion constructs in a stable CAP cell line.

FIG. 4 shows a western blot demonstrating expression of fusion constructs in CAP cell derived EVs.

FIG. 5 shows a western blot demonstrating expression of fusion constructs in producer cell derived EVs.

FIG. 6 shows EV fraction specific nanoluc signalling after size exclusion confirming quality of engineering of the albumin-display EVs.

FIG. 7 shows the in vivo circulation of albumin-display EVs over time.

FIG. 8. shows the in vivo accumulation of albumin-display EVs in various target organs: (a) brain, (b) ILN, (c) liver, (d) lung, (e) spleen, (f) kidney.

### DETAILED DESCRIPTION

The present invention relates to EVs comprising an albumin protein present on the surface of the EVs. Suitably, the albumin is displayed on the surface and the disclosure relates to albumin-display EVs. The present invention also relates to methods of making and purifying the EVs as disclosed herein and their use in therapy.

The EVs of the present invention have several distinct advantages due to the presence of albumin on their surface. Principally, the albumin present on the surface of the EVs can extend the half-life of the EVs in circulation. This extension of half-life can be applied broadly across any, and all, engineered EVs. For example, EVs loaded with any cargo and any targeting moiety as described herein exhibit an improved half-life. Previous attempts to increase half-life of biologics have required specific tailoring to the biologic in question.

The present invention is remarkable in that it is applicable not to the therapeutic cargo, but to the delivery vesicle, thus making it extremely adaptable to increase the half-life of any cargo molecule that is capable of being loaded into or onto the EV. Without wishing to be bound by theory, the increased half-life of albumin-display EVs can also result in altered biodistribution of the albumin-display EVs. This alteration of EV biodistribution, and therefore the pharmacokinetics of the drug cargo carried in or on the EV, is critically important for prolonging the circulation time of the EV and improving the opportunities for targeted delivery of therapeutic EVs. Without wishing to be bound by theory, this results in greater therapeutic efficacy, due to higher payloads being delivered to the target organ. For instance, albumin-display EVs are ideally suited for targeting the brain. Albumin-display EVs are also believed to have improved storage stability because of the protection afforded by the albumin on the surface of the EV. By exploiting a protein such as albumin, all of the aforementioned drawbacks of known methods to increase the half-life of EVs, such as PEGylation, can also be avoided.

The use of an EV according to the disclosure, wherein albumin is fused to an EV protein, is particularly advantageous in the context where the cargo carried is an oligonucleotide, such as an siRNA, with a cholesterol tag. There is a known problem in the EV field caused by dissociation of cholesterol tagged oligonucleotides from EVs due to competition of binding with serum albumin. Utilising albumin-fusion EVs, such as those described herein, means that the cholesterol tagged oligonucleotide will be more stably loaded into/onto the EV because the most proximal albumin to the EV will not exchange with the serum due to it being part of the Alb-EV protein fusion protein. Thus, the issue of serum albumin causing dissociation of the oligonucleotide cargo is reduced and more cargo remains bound to the EV (both in storage and once delivered to the patient) meaning that each EV has greater therapeutic effect when albumin is present as part of an albumin-EV protein fusion protein.

For convenience and clarity, certain terms employed herein are collected and described below. Unless otherwise defined, all technical and scientific terms used herein have the same meaning as commonly understood by one of ordinary skill in the art to which this invention belongs.

In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

“About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “at least one” as used herein can mean at least two, at least three, at least four, at least five, at least six, at least seven, at least eight, at least nine, at least ten or more.

As used herein, the terms, “half-life” and “T½”, can mean the time it takes for the blood plasma concentration of a substance to halve its steady-state (plasma half-life) when circulating in the full blood of an organism.

The terms “extracellular vesicle” or “EV” are used interchangeably herein and shall be understood to relate to any type of vesicle that is obtainable from a cell in any form. The size of EVs may vary considerably, but an EV typically has a nano-sized hydrodynamic radius, i.e., a radius below 1000 nm. Evs can be broadly divided into two categories, (1) ectosomes and (2) exosomes. Some examples of EVs include for instance a microvesicle, an exosome, an apoptotic body, ARMMs, a microparticle, an ectosome, or a cardiosome, etc. Essentially, the terms ‘extracellular vesicle’ and/or ‘EV’ may relate to any type of lipid-based structure (with vesicular morphology or with any other type of suitable morphology) that can act as a delivery or transport vehicle or that has native therapeutic or pharmacological effects.

Furthermore, the said terms shall be understood to also relate to, in some embodiments, extracellular vesicle mimics, cellular membrane vesicles obtained through membrane extrusion, sonication or other techniques, etc.

EVs may be derived from any cell type, whether in vivo, ex vivo or in vitro (further details of suitable source or producer cells are herein described below).

Exosomes, microvesicles and ARRDC1-mediated microvesicles (ARMMs) are just some examples of the different subtypes that fall under the hereinbefore broader description of EVs and represent particularly preferable EVs, but it will be appreciated that other EVs may also be advantageous in certain circumstances.

The terms “apoptotic body” or “apoptotic bodies” are used interchangeably herein and shall be understood to relate to any type of vesicle that is obtainable or derivable from apoptotic cells. Typically, an apoptotic body has a size range of from 1 μm to 5 μm.

The terms “cardiosome” or “cardiosomes” are used interchangeably herein and shall be understood to relate to any type of vesicle that is obtainable or derivable from cardiac cells.

The terms “ectosome” or “ectosomes” are used interchangeably herein and shall be understood to relate to any type of vesicle that is obtainable or derivable from outward budding of the plasma membrane and/or cell membrane of a cell, preferably from neutrophils and monocytes in serum. Examples of Ectosomes include but are not limited to microvesicles, microparticles and large vesicles. Typically, ectosomes range in size from about 50 nm to 1 μm.

The terms “microparticle” or “microparticles” are used interchangeably herein and shall be understood to relate to any type of vesicle that is obtainable or derivable from platelets.

The terms “microvesicles”, “micro-vesicles” and “micro vesicles” are used interchangeably herein and shall be understood to relate to any type of vesicle that is obtainable or derivable or shed from the plasma membrane or cell membrane of a cell.

The terms “ARRDC1-mediated microvesicles” and “ARMMs” are used interchangeably herein and shall be understood to relate to any type of vesicle that is obtainable or derivable from the plasma membrane or cell membrane of a cell, from which they bud directly. Such microvesicles are mediated by the arrestin domain containing protein 1 [ARRDC1] and typically lack known late endosomal markers. As such, ARMMs are distinct from exosomes hereinbefore described.

The terms “exosome” or “exosomes” are used interchangeably herein and shall be understood to relate to any type of vesicle that is obtainable or derivable from the endosomal, lysosomal and/or endo-lysosomal pathway and/or from the plasma membrane, cell membrane or any other membrane of a cell. Exosomes often have a size of from about 30 and 300 nm, typically in the range from 40 and 250 nm, and sometimes from about 40 to 160 nm, which is a highly suitable size range.

The term “modified” indicates that the vesicle has been modified either using genetic or chemical approaches, for instance via genetic engineering of the EV-producing cell, preferably an exosome-producing cell or via e.g., chemical conjugation, for instance to attach moieties to the EV, preferably the exosome surface.

The terms “genetically modified” and “genetically engineered” are used interchangeably herein and indicates that the EV, preferably an exosome is derived from a genetically modified/engineered cell or is otherwise genetically engineered to express, as part of the EV, preferably an exosome, a recombinant fusion protein product which is typically incorporated into the EVs, preferably exosomes, produced by those cells.

Furthermore, the said terms shall be understood to also relate to, in some embodiments, extracellular vesicle mimics, cellular membrane vesicles obtained through membrane extrusion, sonication or other techniques, etc.

The terms “EV protein” “EV polypeptide”, “EV carrier protein” are used interchangeably herein and shall be understood to relate to any suitable protein naturally derived and/or expressed and enriched in an EV as herein defined. The term shall be understood as comprising any polypeptide that enables transporting, trafficking, or shuttling of a fusion protein construct to a vesicular structure, such as an EV. Where embodiments relate to EVs it will understood that the corresponding EV proteins will apply. An EV protein as described herein can therefore be engineered to form one part of a fusion protein capable of transporting another part of the same fusion protein (here, an albumin protein, optionally with a cargo and/or targeting moiety) to the extravesicular membrane of an EV. Examples of EV proteins include transmembrane proteins, preferably multi-pass transmembrane protein and other hallmark EV membrane associated proteins, such as, but not limited to MMP2 and CK18.

The terms “exosome protein”, “exosomal protein”, “exosomal polypeptide”, “exosomal carrier protein” are used interchangeably herein and shall be understood to relate to any suitable protein naturally derived and/or expressed and enriched in an exosome compared to other vesicles/organelles/parent cell. The terms shall be understood as comprising any polypeptide that enables transporting, trafficking or shuttling of a fusion protein construct to an exosome. Where preferred embodiments relate to exosomes it will understood that the corresponding exosomal proteins will apply. An exosomal protein as described herein can therefore be engineered to form one part of a fusion protein capable of transporting another part of the same fusion protein (here, an albumin protein, optionally with a cargo and/or targeting moiety) to the extravesicular membrane of an exosome. Examples of exosomal proteins include transmembrane proteins, preferably multi-pass transmembrane proteins and other hallmark exosomal membrane associated proteins, such as, but not limited to CD81, CD9 and CD63.

The terms “N-terminus”, “N terminal”, “N-terminal domain” and “NTD” are used interchangeably herein and shall be understood to be accorded the conventional meaning in the field unless otherwise indicated.

The terms “C-terminus”, C-terminal”, C-terminal domain” and “CTD” are used interchangeably herein and shall be understood to be accorded the conventional meaning in the field unless otherwise indicated.

The term “Albumin-display EV” shall be understood to refer to EVs as described herein modified to comprise an albumin protein present on the surface of the EV.

A “nucleic acid” refers to a polynucleotide and includes polyribonucleotides and poly-deoxyribonucleotides. Nucleic acids according to the present invention may include any polymer or oligomer of pyrimidine and purine bases, e.g., cytosine (C), thymine (T) and uracil (U), and adenine (A) and guanine (G), respectively (see Albert L. Lehninger, Principles of Biochemistry, at 793-800 (Worth Pub. 1982) and G. Michael Blackburn, Michael J. Gait, David Loakes and David M. Williams, Nucleic Acids in Chemistry and Biology 3rd edition, (RSC publishing 2006), which are herein incorporated in their entirety for all purposes). Indeed, the present invention contemplates any deoxyribonucleotide or ribonucleotide component, and any chemical variants thereof. The polymers or oligomers may be heterogeneous or homogeneous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” can mean a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present invention may be a peptide nucleic acid (PNA; see U.S. Pat. No. 6,156,501, which is hereby incorporated by reference in its entirety.) The invention also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

The term “polynucleotide” includes, for instance, cDNA, RNA, DNA/RNA hybrid, antisense RNA, siRNA, mRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also contemplated are alterations of a wild-type or synthetic gene, including, but not limited to, deletion, insertion, substitution of one or more nucleotides, or fusion to other polynucleotide sequences.

The term “PUF proteins” encompass all related proteins and domains of such proteins (which may also be termed “PUM proteins”), for instance, human Pumilio homolog 1 (PUM1), PUM×2 or PUF×2, which are duplicates of PUM1, etc., or any NA-binding domains obtainable from any PUF (PUM) proteins.

The term “cargo” shall be understood to relate to either a diagnostic cargo or a therapeutic cargo or any combination thereof. Diagnostic cargo and therapeutic cargo are hereinafter defined.

The term “diagnostic cargo” shall be understood to relate to any cargo that might suitably employed in a diagnostic application of the EVs according to the present invention. Examples of suitable diagnostic cargo include but is not limited to radiolabels, fluorescent labels, (bio)luminescent labels and reporters.

The term “therapeutic cargo” and “drug cargo” are used interchangeably herein and shall be understood to relate to any large molecule or small molecule cargo designed for the treatment and/or prophylaxis of a condition, disease and/or disorder. Large molecules and small molecules are hereinafter defined.

The term “therapeutic protein” shall be understood to relate to cargo that is specifically of protein or peptide origin and designed for the treatment and/or prophylaxis of a condition, disease and/or disorder. In addition to including large molecules and small molecules as hereinafter defined, therapeutic proteins may also include proteins and peptides that are not directly therapeutic per se but are endogenously active in the subject, such that they impart an indirect therapeutic effect. Endogenous activity is hereinafter defined.

The terms “large molecule”, “large molecule cargo”, “large molecule drug” or “biologic” and “biologics” are used interchangeably herein and shall be understood to relate to any large molecular agent (as can be defined by MW), nucleic acid, protein, polypeptide, or polysaccharide, which may be used for the treatment, prophylaxis and/or diagnosis of a condition, disease and/or disorder. For the purposes of this invention, a large molecule is typically larger than 900 g/mol, for instance 1500 g/mol, 3000 g/mol, or occasionally even larger and possibly up to about 150.00 Da. Typically, the traditional route of administration to a subject for such large molecules is by injection.

The terms, “small molecule”, “small molecule cargo”, “small molecule drug” and “small molecule therapeutic” are used interchangeably herein and shall be understood to relate to any chemical or small molecular agent (as can be defined by MW), short peptide chain, monosaccharides, disaccharides, other small chain saccharides with a MW of less than 900 Da, sdAbs and Ab fragments with a MW of less than 900 Da, or amino acids, which may be used for the treatment, prophylaxis and/or diagnosis of a condition, disease and/or disorder. Small molecule agents are normally synthesized via chemical synthesis means, but may also be naturally derived, for instance via purification from natural sources, or may be obtained through any other suitable means or combination of techniques. A brief definition of a “small molecule” is any organic compound with a molecular weight of less than 900 g/mol (Dalton) that may help to regulate a biological process. The route of administration to a subject for such small molecules vary but typically include oral administration.

The terms “producer cell”, “cell source”, EV-producing cells” and “EV-producing cell source” are used interchangeably herein and shall be understood to relate to any cell from which the EVs of the present invention may be obtainable or derivable. Generally, EVs may be derived from essentially any cell source, be it a primary cell source or an immortalized cell line. The EV source cells may thus be any embryonic, fetal, or adult somatic stem cell types, including induced pluripotent stem cells (iPSCs) and other stem cells derived by any method, as well as any adult cell source. The source cells per the present invention may be selected from a wide range of cells and cell lines, for including but not limited to mesenchymal stem or stromal cells (obtainable from e.g., bone marrow, adipose tissue, Wharton's jelly, perinatal tissue, chorion, placenta, tooth buds, umbilical cord blood, skin tissue, etc.), fibroblasts, amnion cells and more specifically amnion epithelial (AE) cells optionally expressing various early markers, myeloid suppressor cells, M2 polarized macrophages, adipocytes, endothelial cells, fibroblasts, etc.

The term “endogenously loaded” and “endogenous loading” are used interchangeably herein and shall be understood to relate to any means of loading a desirable cargo, and/or fusion construct into an EV (e.g., exosome) by utilising existing internal mechanisms of a biological system, such as a producer cell, to produce EVs comprising the cargo and/or fusion protein of interest inside the cell. It will be appreciated that nucleic acid constructs might be exogenously loaded into a producer cell, but the resulting polypeptide derived from the engineered nucleic acid construct is made utilising the existing internal mechanisms of said biological system (e.g., cell) using material naturally available within the cell to generate the engineered fusion protein by natural means.

The term “exogenously loaded” and “exogenous loading” are used interchangeably herein and shall be understood to relate to any means of loading a desirable cargo and/or construct into an EV utilising a means that is external to the EV (e.g., exosome). Examples of exogenous loading include, but are not limited to, passive co-incubation, electroporation and transfection.

The terms “population of EVs”, and “EV population” are used interchangeably and shall be understood to encompass a homogenous set of individual EVs. A population of EV may therefore share, for example, the same albumin-display profile, the same therapeutic cargo and/or the same EV protein. In other words, individual EVs, when present in a plurality and having a shared characteristic in common, constitute an EV population.

The terms “population of exosomes”, “exosomal population” and “exosome population” are used interchangeably and shall be understood to encompass a homogenous set of individual exosomes. A population of exosomes may therefore share, for example, the same albumin-display profile, the same therapeutic cargo and/or the same exosome protein. In other words, individual exosomes, when present in a plurality and having a shared characteristic in common, constitute an exosome population.

The term “subject” refers to any animal to which the therapeutic EVs according to the present invention are administered. Typically, the subject is afflicted with or susceptible to be afflicted with a condition, disease and/or disorder, the treatment/prophylaxis or diagnosis of which would benefit from a therapeutic EV according to the present invention. Preferably the subject is a mammal, more preferably human.

The term “endogenously active” and “endogenous activity” is used interchangeably herein and shall be understood to relate to the therapeutic activity of the therapeutic EVs and/or their cargo, specifically protein cargo, according to the present invention in a subject wherein the therapeutic EVs cause the tissues and/or cells of a subject as hereinbefore described to generate their own means for the treatment or prophylaxis of a condition disease and/or disorder utilising the internal mechanisms of the tissues and/or cells of the subject to produce said means.

It will be appreciated that said means could be a drug cargo, large molecule or small molecule based therapeutic, or protein cargo as herein described.

Additionally, it should be noted that embodiments and features described in connection with one of the aspects and/or embodiments of the present invention also apply mutatis mutandis to all the other aspects and/or embodiments of the invention. For example, the albumin proteins, fragments and domains thereof and albumin based fusion proteins/polypeptides described herein in connection with the EVs are to be understood to be disclosed, relevant and compatible with all other aspects, teachings and embodiments herein, for instance aspects and/or embodiments relating to the methods for producing or purifying the EVs or relating to the corresponding polynucleotide constructs described herein or the engineered EV-producing cells from which the EVs derive. Furthermore, certain embodiments described in connection with certain aspects, for instance the administration routes of the EVs comprising the therapeutic cargo molecule and optionally the fusion polypeptides, as described in relation to aspects pertaining to treating certain medical indications, may naturally also be relevant in connection with other aspects and/or embodiments, such as those pertaining to the pharmaceutical compositions comprising such EVs. Furthermore, all polypeptides and proteins identified herein can be freely combined in fusion proteins using conventional strategies for fusing polypeptides. As a non-limiting example, albumin protein described herein may be freely combined in any combination with one or more EV proteins, optionally combined with all other polypeptide domains, regions, sequences, peptides, groups herein, e.g., any multimerization domains, linker sequences, release domains, therapeutic cargo molecules, endosomal escape domains and/or targeting moieties. Moreover, any and all, features can be freely combined with any and all other features, e.g., any albumin may be combined with any EV protein. Furthermore, when teachings herein refer to EVs in singular and/or to EVs as discrete natural nanoparticle-like vesicles, it shall be understood that all such teachings are equally relevant for and applicable to a plurality of EVs and populations of EVs. As a general remark, albumin binding domains, the EV proteins, the EV-producing cell sources, the additional domains and moieties, the therapeutic cargo molecules, the targeting moieties and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides, and sequences, provided any given molecule retains the ability to carry out the desired technical effect associated therewith. Provided their biological properties are maintained, the polypeptide and/or polynucleotide sequences according to the present application may deviate with typically as much as 50% and in some instances as much as 30% (calculated using, for instance, BLAST or ClustalW) as compared to the native sequence, although a sequence identity or similarity that is as high as possible is preferable (for instance at least 60%, at least 70%, at least 80%, or e.g., at least 90% or higher).

Standard methods in the art may be used to determine sequence identity or homology. For example, PILEUP and BLAST algorithms can be used to calculate homology or line up sequences. The combination (fusion) of e.g., several polypeptides implies that certain segments of the respective polypeptides may be replaced and/or modified and/or that the sequences may be interrupted by insertion of other amino acid stretches, meaning that the deviation from the native sequence may be considerable provided the key properties (e.g., ability to extend half-life, ability to traffic a fusion construct to an EV, targeting capabilities, etc.) are conserved. Similar reasoning thus naturally applies to the polynucleotide sequences encoding for such polypeptides. Any SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein, but also variants and derivatives thereof. All proteins, polypeptides, peptides, nucleotides, and polynucleotides mentioned herein are to be construed according to their conventional meaning as understood by a skilled person, unless otherwise defined.

An EV according to the invention comprises an albumin protein as described herein present on the surface of the EV.

Albumins are a family of globular proteins, the most common of which are the serum albumins. All the proteins of the albumin family are water-soluble. Typically, albumin is produced by the liver and serves to retain bodily fluids in the bloodstream so that it does not leak into the surrounding tissue. Albumins may be engineered by specific mutagenesis to achieve increased stability, lower immunogenicity or an improved binding affinity. One advantage of albumin protein is that improves the half-life of the therapeutic EV, preferably exosome. The inventors have surprisingly found that albumin protein protects the therapeutic EV or exosome from degradation on in vivo circulation within a subject. Another advantage of albumin is that it is generally well tolerated by a subject in vivo post administration. Certain albumin proteins are also capable of binding to the recycling receptor FcRN, which is involved in receptor mediated endocytosis. As such, uptake of the EV or exosome into target cells and/or tissue is enabled.

An albumin protein as described herein may be a recombinant albumin protein or an albumin protein derived from any species or a fragment, variant, domain or derivative thereof as described herein. In a preferred aspect, the albumin protein is human serum albumin (HSA), for example an albumin protein having the amino acid sequence of SEQ ID NO:1; or a fragment of HSA, such as human serum albumin domain three (DIII), for example an albumin protein having the amino acid sequence of SEQ ID NO:2.

Albumin has a heart-like structure meaning that it is capable of tessellating with another albumin. Albumin is also capable of oligomerising. It will be appreciated that this capability would also extend to albumin variants that have a similar shape or another structure that is capable of being tessellated such that oligomer formation is enabled.

Exemplary Albumin sequences used in the present invention include: SEQ ID NO:1 (human serum albumin) and SEQ ID NO:2 (human serum albumin domain three (DIII)), nevertheless other albumin proteins may equally be employed. It will be appreciated that sequences may be either immature or mature. Immature sequences may comprise additional sequences useful in the processing of the polypeptide, such as signal protein (SP) and propeptide.

Mature sequences comprise the polypeptide in its final form (i.e., without the processing sequences such as the SP and propeptide). The present invention encompasses fragments, domains, variants or derivatives of these sequences, which have at least a 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity or homology to these sequences but retain the biological activity of the albumin protein. Suitably, certain fragments, domains, variants or derivatives of the albumin proteins described herein are capable of binding to the recycling receptor FcRN enabling uptake of the EV or exosome into target cells and/or tissue. Suitably, certain fragments, variants or derivatives of the albumin proteins described herein are capable of tessellation and/or oligomerization with another albumin protein as described herein. One advantage of using a fragment of albumin protein is that it is sufficiently small (for example DIII) so that addition of further constructs (e.g., another albumin protein, cargo and/or targeting moiety) is more readily loaded into the same fusion protein. Another advantage of using a fragment of an albumin protein (such as a specific domain) is that the fusion protein construct can be designed as small as possible so it will be well tolerated by cells transfected to express the fusion construct. This reduces the stress on the cells and allows the generation of single and even double stable cell lines. Stable cells lines are essential to the production of clinical grade pharmaceutical products.

The present invention relates to EVs, preferably an exosome comprising an albumin protein present on the surface of the EVs or preferably an exosome, wherein the albumin has about 30%, 40%, 50%, 60%, 70%, 80%, 90% or 95% sequence identity or homology to any of SEQ ID NOs:1-2. The albumin may be synthesized using standard peptide synthesis methods known in the art. In one aspect, an albumin protein as described herein has an amino acid sequence having at least 33.7%, at least 34.1% or at least 35.5% sequence identity with SEQ ID NO:1.

The albumin protein as disclosed herein is displayed on the surface of the EVs. The albumin protein may be presented on the surface of the EV in any number of ways known to the skilled person, provided that the albumin protein is exposed on the outer surface of the EV. Full-length albumin proteins have been used for increasing the half-life of biologics. However, such an approach has never been utilised for EVs, preferably an exosome. EVs (or exosomes) incorporating an albumin protein as described herein and methods for successfully incorporating an albumin protein into the surface of an EV, or preferably an exosome, such that the albumin protein is displayed on the surface of an EV, preferably an exosome, have not been described previously. The inventors have successfully incorporated an albumin protein such that it imparts a protective effect to an EV (such as an exosome), despite the challenges posed by the differing size and/or charge profile and/or lipid profile of EVs, preferably an exosome. The Inventors have surprisingly found that incorporating an albumin protein into an EV, or exosome, improves the half-life of the EV or exosome in circulation and positively impacts the biodistribution profile of said EV or exosome. The inventors teach how to produce such an advantageous albumin-display EV and/or exosome.

Commonly the albumin will form part of a fusion protein with an EV protein.

One advantage of including an EV protein as part of a fusion with the albumin protein is that the EV protein enables the albumin protein to be actively loaded into each EV because the presence of the EV protein actively drives loading of the fusion construct into each EV.

Another advantage of including an EV protein as part of a fusion with the albumin protein is that the EV protein enables the albumin protein to be displayed on the surface of the EV (i.e., to be present on the outside of the EV).

Another advantage of including an EV protein as part of a fusion protein with the albumin protein is that natural incorporation of the albumin into the EV is enabled without any need for conjugation steps (i.e., complexity of incorporation is reduced) after the EV is purified.

Another advantage of including an EV protein as part of a fusion protein with the albumin protein is that the EV retains all the benefits of being naturally derived such as retaining the corona proteins and natural morphology.

An EV according to the invention can comprise an EV protein as part of a fusion protein with the albumin protein as described herein. An EV protein according to various aspects can be an EV transmembrane protein or an EV membrane associated protein. Inclusion of an EV protein as part of a fusion with an albumin protein enables the albumin protein to be actively loaded into the EVs because the presence of the EV protein actively drives loading of the fusion construct into the EV.

In one aspect, the EV protein is an EV protein associated with the outer surface of the membrane. In one aspect, the EV protein is a transmembrane protein, such as a single-pass transmembrane protein or a multi-pass transmembrane protein. Preferably, the EV protein is an exosomal protein.

The EV protein which is comprised in a fusion protein as per the present invention may be selected from the group consisting of the following non-limiting examples: CD9, CD53, CD63, CD81, CD54, CD50, FLOT1, FLOT2, CD49d, CD71, CD133, CD138, CD235a, AAAT, AT1B3, AT2B4, ALIX, Annexin, BASI, BASP1, BSG, Syntenin-1, Syntenin-2, Lamp2, Lamp2a, Lamp2b, TSN1, TSN3, TSN4, TSN5 TSN6, TSN7, TSPAN8, TSN31, TSN10, TSN11, TSN12, TSN13, TSN14, TSN15, TSN16, TSN17, TSN18, TSN19, TSN2, TSN4, TSN9, TSN32, TSN33, TNFR, TfR1, syndecan-1, syndecan-2, syndecan-3, syndecan-4, CD37, CD82, CD151, CD224, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11 b, CD11c, CD18/ITGB2, CD41, CD49b, CD49c, CD49e, CD51, CD61, CD104, CLIC1, CLIC4, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD53, CD86, CD110, CD111, CD115, CD117, CD125, CD135, CD184, CD200, CD279, CD273, CD 274, CD362, COL6A1, AGRN, EGFR, FPRP, GAPDH, GLUR2, GLUR3, GP130, GPI anchor proteins, GTR1, HLAA, HLA-DM, HSPG2, ITA3, Lactadherin, L1 CAM, LAMB1, LAMC1, LIMP2, MYOF, ARRDC1, ATP2B2, ATP2B3, ATP2B4, BSG, IGSF2, IGSF3, IGSF8, ITGB1, ITGA4, ATP1A2, ATP1A3, ATP1A4, ITGA4, SLC3A2, ATP transporters, ATP1A1, ATP1B3, ATP2B1, LFA-1, LGALS3BP, Mac-1 alpha, Mac-1 beta, MFGE8, a member of the myristoylated alanine rich Protein Kinase C substrate (MARCKS) protein family such as MARCKSL1, matrix metalloproteinase-14 (MMP14), PDGFR, PTGFRN, PRPH2, ROM1, SLIT2, SLC3A2, SSEA4, STX3, TCRA, TCRB, TCRD, TCRG, TFR1, UPK1A, UPK1B, VTI1A, VTI1B, and any other EV proteins, and any combinations, derivatives, domains, variants, mutants, or regions thereof. Mutations may be introduced into the wild-type sequence of the EV protein to alter its function. A preferred mutant according to the present invention is CD63(Y235A). Without wishing to be bound by theory, the use of EV proteins has the effect of driving loading of the albumin into EVs, such that albumin is actively loaded into EVs as hereinbefore described. This process of actively loading is often referred to as endogenous loading and has several benefits. For example, it allows the natural incorporation of the albumin into the EV without any need for conjugation steps after the EV is purified and ensures the EV retains all the benefits of being naturally derived such as retaining the corona proteins and natural morphology. As a result of their ability to actively load other components into the EV, the EV proteins are sometimes referred to as carrier proteins.

Particularly advantageous EV proteins include tetraspanins, such as TSPAN2, TSPAN3, CD63, CD81, CD9, CD82, as well as CD44, CD47, CD55, LAMP2B, LIMP2, ICAMs, integrins, ARRDC1, syndecan, syntenin, TNFR, TfR1, and Alix, as well as derivatives, domains, variants, mutants, or regions thereof.

In one aspect, the EV protein is selected from one or more of, CD82, CD44, CD47, CD55, LAMP2B, TNFR, Tfr1, LIMP2, ICAMs, ARRDC1, and derivatives, domains, variants, mutants, or regions thereof. In a preferred aspect, the EV protein is LAMP2B or LIMP2.

In one aspect, the EV protein is a transmembrane protein comprising at least two, three, four, five, six, seven or eight transmembrane domains. In one aspect, the transmembrane protein comprises four transmembrane domains and two extracellular loops. In one aspect, the transmembrane protein is a tetraspanin. In one aspect, the tetraspanin is selected from one or more of CD36, CD9, CD53, CD63, CD81, CD151 or any one or more of TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, TSPAN20, TSPAN21, TSPAN22, TSPAN23, TSPAN24, TSPAN25, TSPAN26, TSPAN27, TSPAN28, TSPAN29, TSPAN30, TSPAN31, TSPAN32, TSPAN33 and derivatives, domains, variants, mutants, or regions thereof.

One advantage of utilising a transmembrane protein, particularly a multi-pass transmembrane protein (such as a tetraspanin), is that more than one albumin protein may be incorporated into the fusion protein without affecting the expression of the transmembrane protein as the transmembrane protein is still able to correctly fold and form, meaning that the transmembrane protein is more stable and thus the albumin protein is more likely to remain stably introduced into the EV protein. This is especially the case with multi-pass transmembrane proteins such as tetraspanins which possess multiple loops on the surface of the EV that allow multiple points for engineering the albumin protein into the EV protein and by extension into the EV (e.g., an exosome).

Another advantage expressing more than one albumin in the transmembrane EV protein is that more albumin protein can be bound to the EV per fusion protein expressed, thus increasing the amount of albumin coating the EV and thus increasing the shielding effect of the albumin. This, in turn, increases the half-life of the albumin-display EV.

An EV of the present invention can comprise an albumin protein present on the surface of the EV, wherein the albumin protein is fused or engineered into an extravesicular loop or loops of a multi-pass transmembrane protein, for example Limp2. In one aspect, the multi-pass transmembrane protein is preferably a tetraspanin such as CD36, CD9, CD81 or any of TSPAN1-TSPAN33. The albumin protein may be fused into the first, second, third, fourth or any subsequent loop of the multi-pass transmembrane EV protein or into more than one of the loops. Suitably the albumin protein is fused into the first, second or any subsequent loop of the multi-pass transmembrane EV protein. Advantageously, albumin proteins may be incorporated into more than one of the loops of said multi-pass transmembrane protein and/or more than one albumin protein i.e., a plurality of albumin proteins, may be incorporated into each loop of said multi-pass transmembrane protein. The plurality of albumin proteins present in the loop or loops may be the same or different albumin proteins.

An advantage of utilizing the various loop portions of the EV protein as herein described is that the albumin protein may be incorporated into more than one of the loops of said multi-pass transmembrane protein and/or more than albumin may be incorporated into each loop of said multi-pass transmembrane protein.

Another advantage of utilizing the various loop portions of the EV protein as hereinabove described is that, where an albumin fragment or variant is used, more than one albumin protein may be incorporated into the transmembrane fusion protein without affecting the expression of the transmembrane protein itself or the folding and form, meaning that the transmembrane protein is more stable and the albumin protein is more likely to remain stably introduced into the extravesicular loop or loops of the EV protein.

In a preferred embodiment, an EV as disclosed herein comprises an albumin protein engineered into one or more extravesicular loop of a multi-pass transmembrane protein, and the multi-pass transmembrane protein is a tetraspanin or Limp2.

In another aspect, the transmembrane protein is a single-pass transmembrane protein and display of the albumin protein is by terminal display, such as C-terminal display or N-terminal display. In one aspect, the albumin protein may be fused to the N terminal domain (NTD) or C terminal domain (CTD) of the EV protein.

The presence of more than one albumin protein per transmembrane EV protein means that more albumin molecules can be bound to the EV per fusion protein expressed. This is advantageous as it increases the amount of albumin coating the EV and thus increases the shielding effect of the albumin protein. This, in turn, increases the half-life of the albumin-display EVs, whilst allowing the additional constructs to be expressed in the same EV, for instance, additional constructs may comprise therapeutic cargos and/or targeting moieties.

The EV may comprise more than one albumin, i.e., a plurality of albumins. The plurality of albumins may be the same or different from one another.

One advantage of the presence of more than one albumin protein on a single EV is that it increases the amount of albumin protein coating the EV and thus increases the shielding effect of the albumin protein, which in turn can increase the half-life of the albumin-display EV. The albumin protein can form oligomers on the surface of the EV, thus adding strength to the albumin shield.

One advantage of the plurality of albumins being different to one another is that different structural arrangements are enabled, such that the shielding effect of the albumin protein can be tailored and thus increase the extent of the shielding effect of the albumin protein in discrete positions about the surface of the EV, preferably exosome. This in turn can increase the half-life of the albumin-display EV through protecting the most vulnerable parts of the EV (e.g., exosome) that are most susceptible to being degraded.

Engineered EVs according to the present invention may be produced in a single stable or double stable cell line. Stable cells lines are essential to the production of clinical grade pharmaceutical products. The generation of single stable cells lines is simpler and more reliable. As such the ability to employ single stable cells lines is desirable. The ability to introduce a second construct (i.e., generate a double stable cell line) means that other constructs encoding for cargo or targeting moieties can be added to the cell line and thus incorporated into the resulting EV. The present invention includes the full albumin protein or an albumin fragment in the fusion construct. Albumin has a MW of 66.5 kDa but can be modified to be smaller. Without wishing to be bound by theory, the smaller size of the albumin protein (e.g., DIII) allows for simpler generation of stable cell lines, as the transmembrane EV protein is still able to correctly fold and form, meaning that the transmembrane EV protein is more stable and thus the albumin protein is more likely to remain stably introduced into the EV protein. A smaller albumin protein also enables more copies of the albumin protein to be fused into the fusion polypeptides of the invention. Smaller albumins are also beneficial because the small size is better for allowing other proteins, such as cargo proteins and/or targeting moieties, to be included within the same fusion construct. Without wishing to be bound by theory, this can allow the albumin-display EVs to act as a highly flexible platform from which any number of different therapeutic EVs can be produced because the albumin-display EVs are able to easily be modified to accommodate more cargo/s and/or targeting moieties.

The EVs of the present invention can comprise additional sequences or domains within the fusion protein construct as described herein comprising an albumin protein and an EV protein (also referred to herein as an “albumin EV fusion protein”). In one advantageous embodiment, the albumin EV protein further comprises at least one multimerization domain.

Multimerization domains according to the present invention may be homomultimerization domains or heteromultimerization domains. The multimerization domains of the present invention may be dimerization domains, trimerization domains, tetramerization domains, or any higher order of multimerization domain. Without wishing to be bound by theory, multimerization domains can enable dimerization, trimerization, or any higher order of multimerization of the fusion polypeptides, which increases the sorting and trafficking of the fusion polypeptides into EVs and may also contribute to increasing the yield of vesicles produced by EV-producing cells. Exemplary multimerization domains include but are not limited to the following: leucine zipper, fold-on domain, fragment X, collagen domain, 2G12 IgG homodimer, mitochondrial antiviral-signaling protein CARD filament, Cardiac phospholamban transmembrane pentamer, parathyroid hormone dimerization domain, Glycophorin A transmembrane, human immunodeficiency virus (HIV) Gp41 trimerisation domain, HPV45 oncoprotein E7 C-terminal dimer domain, and any combination thereof.

In another advantageous embodiment, the albumin EV protein fusion protein further comprises at least one endosomal escape domain. Endosomal escape domains can include: HA2, VSVG, GALA, B18. Other exemplary endosomal escape domains include, but are not limited to: HIV TAT PDT (peptide/protein transduction domain), HIV Gp-120, KALA, GALA and INF-7 (derived from the N-terminal domain of influenza virus hemagglutinin HA-2 subunit), endosomal escape moieties that act by causing membrane fusion such as Diphtheria toxin T domain, proton sponge type endosomal escape moieties such as peptides or lipids with histidine or imidazole moieties and cell penetrating peptides (CPPs) and other moieties that enable endosomal escape. As would be appreciated by the skilled person, CPPs are typically less than 50 amino acids, but may also be longer, are typically highly cationic and rich in arginine and/or lysine amino acids and have the ability to gain access to the interior of virtually any cell type. Exemplary CPPs are transportan, transportan 10, penetratin, MTS, VP22, CADY peptides, MAP, KALA, PpTG20, proline-rich peptides, MPG peptides, PepFect peptides, Pep-1, L-oligomers, calcitonin peptides, various arginine-rich CPPs, such as poly-Arg, tat and combinations thereof. The presence of endosomal escape domains advantageously assists to drive endosomal escape and thereby enhance the bioactive delivery of the EV per se. Use of endosomal escape strategies can be used in the treatment of diseases where the cargo carried within the EV is required to be delivered into the cytosol of the recipient cell or within any other compartment that is outside of the endo-lysosomal system.

In one aspect, the albumin EV protein fusion protein further comprises at least one linker, spacer and/or scaffold sequence. Advantageously, the presence of linkers, spacers and/or scaffold sequences allows flexibility and enables the albumin protein to be positioned optimally for display on the surface of the EV.

Linkers according to the invention are useful in providing increased flexibility, improving pharmacokinetics (PK), increasing expression and improving biological activity of the fusion polypeptide constructs, and also to the corresponding polynucleotide constructs, and may also be used to ensure avoidance of steric hindrance and maintained functionality of the fusion polypeptides. Exemplary linkers according to the present invention include glycine or serine linkers, which increase stability or flexibility, such as (GGGGS)n (n=1, 2, 4) or (Gly)6, (Gly)8, rigid linkers such as (EAAAK)n (n=1-3, and A(EAAAK)4ALEA(EAAAK)4A, bending linkers (XP)n or cleavable linkers such as disulphide, protease sensitive sequences.

As hereinbefore described, multiple albumin proteins per EV may result from more than one albumin protein being present in a single fusion protein. Alternatively, it may result from multiple fusion proteins being loaded into a single EV. Said multiple fusion proteins may comprise the same or different EV protein. Without wishing to be bound by theory, the presence of more than one albumin on a single EV can be advantageous as it increases the amount of albumin coating the EV and thus increases the shielding effect of the albumin, which in turn can increase the half-life of the albumin EV. In some embodiments, the EVs of the invention comprises at least one albumin EV fusion protein, wherein the at least one albumin EV fusion protein comprises at least one, or at least two, or at least three, or at least four, or at least five, or at least six, or at least seven, or at least eight, or at least nine, or at least ten albumins.

EVs comprising albumin proteins may be produced using any of the methods disclosed herein.

EVs can be further loaded with a cargo, such as a diagnostic cargo, preferably a reporter protein, more preferably either GFP or nanoluc, or a therapeutic cargo. The therapeutic cargo to be loaded according to the present invention may be essentially any type of drug cargo, such as for instance: a nucleic acid such as an RNA molecule, a DNA molecule or a mixmer, mRNA, an antisense or splice-switching oligonucleotide, siRNA, shRNA, miRNA, plasmid DNA (pDNA), a supercoiled or unsupercoiled plasmid, or a mini-circle; a peptide or protein including: a transporter, enzyme, receptor such as a decoy receptor, membrane protein, cytokine, antigen or neoantigen, ribonuclear protein, nucleic acid binding protein, antibody, nanobody or an antibody fragment; an antibody-drug conjugate; a small molecule drug; gene editing technology such as CRISPR-Cas9, a TALEN, meganuclease; or a vesicle-based cargo such as a virus (e.g. an AAV, lentivirus, etc.). In one embodiment, the cargo may be a mixture of protein, nucleic acid, virus, viral genome, antigen, small molecule and/or biologic. In a preferred aspect, the cargo is an oligonucleotide, preferably an siRNA. In another preferred aspect, the cargo is an oligonucleotide with a cholesterol tag, preferably an siRNA with a cholesterol tag. The siRNA can be any of the siRNAs disclosed herein.

In more detail, the nucleic acid cargo molecule of the invention may be selected from the group comprising shRNA, siRNA, saRNA, miRNA, an anti-miRNA, mRNA, gRNA, pri-miRNA, pre-miRNA, circular RNA, piRNA, tRNA, rRNA, snRNA, lncRNA, ribozymes, mini-circle DNA, plasmid DNA, RNA/DNA vectors, trans-splicing oligonucleotides, splice-switching oligonucleotides, CRISPR guide strands, morpholinos (PMO) antisense oligonucleotides (ASO), peptide-nucleic acids (PNA), a viral genome and viral genetic material (for instance, a naked AAV genome), but essentially any type of nucleic acid molecule can be delivered by the EVs of the present invention. Both single-stranded and double-stranded nucleic acid molecules are within the scope of the present invention, and the nucleic acid molecule may be naturally occurring (such as RNA or DNA) or may be a chemically synthesised RNA and/or DNA molecule, which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

Indeed, the present invention contemplates any deoxyribonucleotide or ribonucleotide component, and any chemical variants thereof. The polymers or oligomers may be heterogeneous or homogeneous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The present invention specifically relates to albumin EVs which are further loaded with nucleic acids such as siRNAs, which target oncogenes known to be involved with the development of cancer. The genes targeted by the nucleic acids according to the present invention may be ABL, AF4/HRX, AKT-2, ALK, ALK/NPM, AML1, AML1/MTG8, AXL, BCL-2, 3, 6, BCR/ABL, c-MYC, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HER2/neu, HOX11, HST, IL-3, INT-2, JUN, KIT, KS3, K-SAM, LBC, LCK, LMO1, LMO2, L-MYC, LYL-1, LYT-10, LYT-10/Cα1, MAS, MDM-2, MLL, MOS, MTG8/AML1, MYB, MYH11/CBFB, NEU, N-MYC, OST, PAX-5, PBX1/E2A, PIM-1, PRAD-1, RAF, RAR/PML, RAS-H, RAS-K, RAS-N, REL/NRG, RET, RHOM1, RHOM2, ROS, SKI, SIS, SET/CAN, SRC, TAL1, TAL2, TAN-1, TIAM1, TSC2, TRK. In a preferred aspect, the disclosure relates to albumin EVs which are loaded with nucleic acids, preferably an oligonucleotide, preferably an siRNA. In another preferred aspect, the disclosure relates to albumin EVs which are loaded with nucleic acids, preferably an oligonucleotide with a cholesterol tag, preferably an siRNA with a cholesterol tag. The siRNA can be any of the siRNAs disclosed herein.

In more detail, the therapeutic protein cargos according to the present invention include: antibodies, intrabodies, nanobodies, scFvs, affibodies, bi- and multi-specific antibodies or binders including bispecific T-cell engagers (BiTEs), receptors, ligands, transporters, enzymes for e.g. enzyme replacement therapy (ERT) or gene editing, tumour suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance, pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription-related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins such as cytokines and inhibitors of such cytokines (cytokines may include: CXCL8, GMCSF, interleukins including: IL-1 family, IL-2, IL-4, IL-6, IL-6-like, IL-9, IL-10, IL12, IL-13, IL-17, interferons including INF-alpha/beta/gamma, TNF family members, CD40 and CD40L, TRAIL, and TGF-beta family), mitochondrial proteins, and heat shock proteins, etc. The cargo protein may be a reporter protein such as green fluorescent protein (GFP) or nanoLuc. In one preferred embodiment, the encoded protein is a CRISPR-associated (Cas) polypeptide (such as Cas9) with intact nuclease activity, which is associated with (i.e., carries with it) an RNA strand that enables the Cas polypeptide to carry out its nuclease activity in a target cell once delivered by the peptide. Alternatively, in another preferred embodiment, the Cas polypeptide may be catalytically inactive, to enable targeted genetic engineering. Yet another alternative may be any other type of CRISPR effector such as the single RNA guided endonuclease, Cpf1. The inclusion of Cpf1 is a particularly preferred embodiment of the present invention, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression.

The present invention also relates, in some embodiments, to nucleic acid cargos, which are loaded by fusion of an EV protein to a nucleic acid binding protein (NA-binding protein), which then binds to the nucleic acid cargo molecule and causes loading of the nucleic acid cargo into the EV. Non-limiting examples of NA-binding proteins are hnRNPA1, hnRNPA2B1, DDX4, ADAD1, DAZL, ELAVL4, IGF2BP3, SAMD4A, TDP43, FUS, FMR1, FXR1, FXR2, EIF4A13, the MS2 coat protein, as well as any domains, parts or derivates, thereof. More broadly, specific subclasses of RNA-binding proteins and domains, e.g., mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins, and transcription factors (TFs). Furthermore, various domains and derivatives may also be used as an NA-binding domain to transport a nucleic acid cargo into EVs. Non-limiting examples of RNA-binding domains include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae). Such RNA-binding domains may be present in a plurality, alone or in combination with others, and may also form part of a larger RNA-binding protein construct as such, provided their key function (i.e., the ability to transport a nucleic acid cargo of interest, e.g., an mRNA or a short RNA) is maintained.

In preferred embodiments, the present invention relates to two groups of NA-binding domains, namely PUF proteins and CRISPR-associated polypeptides (Cas), specifically Cas6 and Cas13, as well as various types of NA-binding aptamers.

PUF proteins and regions and derivatives thereof that may be used as NA-binding domains as per the present invention include the following non-limiting list of PUF proteins: FBF, FBF/PUF-8/PUF-6,-7,-10, all from C. elegans; Pumilio from D. melanogaster; Puf5p/Mpt5p/Uth4p, Puf4p/Yg1014wp/Yg1023p, Puf5p/Mpt5p/Uth4p, Puf5p/Mpt5p/Uth4p, Puf3p, all from S. cerevisiae; PufA from Dictyostelium; human PUM1 (Pumilio 1, sometimes known also as PUF-8R) and any domains thereof, polypeptides comprising NA-binding domains from at least two PUM1, any truncated or modified or engineered PUF proteins, such as for instance PUF-6R, PUF-9R, PUF-10R, PUF-12R, and PUF-16R or derivatives thereof; and X-Puf1 from Xenopus. Particularly suitable NA-binding PUFs as per the present invention include the following: PUF 531, PUF mRNA loc (sometimes termed PUFengineered or PUFeng), and/or PUF×2, (sequences of which are available in International Patent Publication No. WO 2019/092145) and any derivatives, domains, and/or regions thereof. The PUF/PUM proteins are highly advantageous as they may be selected to be of human origin.

Furthermore, as is the case with the PUF proteins, Cas proteins such as Cas6 and Cas13 are examples of releasable NA-binding domains, which bind with suitable affinity to nucleic acid cargo molecules, thereby enabling a releasable, reversible attachment of the Cas protein to the nucleic acid cargo. As with the PUF-based NA-binding domains, the Cas proteins represent a releasable, irreversible NA-binding domain with programmable, modifiable sequence specificity for the target nucleic acid cargo molecule, enabling higher specificity at a lower total affinity, thereby allowing for both loading of the nucleic acid cargo into EVs and release of the nucleic acid cargo in a target location.

Additional preferred embodiments include therapeutic protein cargos selected from the group comprising enzymes or transporters for lysosomal storage disorders, for instance, glucocerebrosidases such as imiglucerase, alpha-galactosidase, alpha-L-iduronidase, iduronate-2-sulfatase and idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2 and hexoaminidase.

Additional preferred embodiments include therapeutic protein cargos selected from the group comprising enzymes associated with urea cycle disorders including: N-acetylglutamate synthase, carbamoyl phosphate synthetase, ornithine transcarbamoylase (OTC), argininosuccinic acid synthase, argininosuccinic acid lyase, arginase, mitochondrial ornithine transporter, citrin, y+L amino acid transporter 1 and uridine monophosphate synthase (UMPS).

In other preferred embodiments, the therapeutic protein cargo may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g., myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The therapeutic protein cargos are typically proteins or peptides of human origin unless indicated otherwise by their name, any other nomenclature, or as known to a person skilled in the art, and they can be found in various publicly available databases such as Uniprot, RCSB, etc.

In a more preferred embodiment, the therapeutic cargo is selected from one or more of CD24 and CD52.

In another preferred embodiment, the therapeutic cargo is an antigen/neoantigen, optionally wherein the antigen/neoantigen is suitable for use in cancer immunotherapy.

Any antigen/neoantigen may be incorporated into the EVs of the present invention. The antigens may be suitable for raising immune responses against pathogens, such as bacteria, viruses and funguses, or the antigen may be a tumor antigen useful in eliciting an immune response against a tumor for cancer immunotherapy. There may be one or more antigens/neoantigens present in any EV according to the invention. The one or more antigens/neoantigens may be endogenous/autologous (coming from the subject itself) or exogenous/allogenic (coming from another subject) or, in the case of more antigens/neoantigens being incorporated into/onto the EVs, the antigens/neoantigens may be any mix of autologous/allogenic antigens. Preferably the antigens are autologous. Moreover, the one or more antigens/neoantigens may have any origin, such as e.g., viral or bacterial, or may be a tumour antigen and, furthermore, may be immunostimulatory or immunosuppressive or a combination thereof. The antigen/neoantigen maybe be useful in the treatment of any disease by immunotherapy. The treatment of cancer by immunotherapy is a particularly preferred embodiment. Where the antigen is a neo-antigen, it may be identified by sequencing of a tumour to identify the neo-antigen.

Exemplary tumour antigens include, but are not limited to: alphafetoprotein (AFP), carcinoembryonic antigen (CEA), CA-125, MUC-1, epithelial tumor antigen (ETA), melanoma-associated antigen (MAGE), WT-1, NY-ESO-1, LY6K, IMP3, DEPDC1, CDCA-1, abnormal products of ras, p53, KRAS, or NRAS, CTAG1B, peptides derived from chromosomal translocations such as BCR-ABL or ETV6-AML1, viral antigens such as peptides from HPV-related cancers, peptides derived from proteins such as tyrosinase, gp100/pmelI17, Melan-A/MART-1, gp75/TRP1, or TRP2, and overexpressed antigens such as MOK (RAGE-1), ERBB2 (HER2/NEU).

Where the therapeutic cargo is an antigen or neoantigen, the EV or pharmaceutical composition comprising the EV (discussed further below) may optionally further comprise at least one adjuvant. Where the antigen is administered with an adjuvant to stimulate the immune response, the adjuvant may be, but is not limited to: an inorganic compound, such as aluminium hydroxide, aluminium phosphate, calcium phosphate hydroxide, a mineral oil such as paraffin oil, bacterial products such as killed bacteria Bordetella pertussis, Mycobacterium bovis, toxoids, a nonbacterial organic such as squalene, a detergent such as Quil A, a plant saponin, a cytokine such as IL-1, IL-2, IL-12, or Ribi Adjuvants (muramyl dipeptides) or immunostimulating complexes (ISCOM) such as stimulator of interferon genes (STING) agonists, which can include cyclic dinucleotides. Such adjuvants may protect the therapeutic EV from rapid dispersal by sequestering it in a local deposit, or they may contain substances that stimulate the host to secrete factors that are chemotactic for macrophages and other components of the immune system. Adjuvants that can be incorporated into a vaccine are well-known by a person skilled in the art and will be selected in such a way that they do not negatively affect the immunological activity of the EV.

The present invention also relates to albumin EVs which are loaded with viral cargos, optionally wherein the albumin EV further comprises one or more immune effector molecules that provide immune effector functions. Exemplary viral cargos include, but are not limited to, a viral vector, which is an adeno-associated viral (AAV) vector or a lentiviral vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12.

In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9 or AAV10. In some embodiments, the AAV capsid and the AAV ITR are from the same serotype or from different serotypes.

In some embodiments of the above aspects, the viral vector is a lentiviral vector. In some embodiments, the lentiviral vector is derived from HIV, a simian immunodeficiency virus or a feline immunodeficiency virus. In some embodiments, the lentiviral vector is non-replicating. In some embodiments, the lentiviral vector is non-integrating.

In some embodiments, the viral vector comprises a viral capsid and a viral genome, the viral genome comprising one or more heterologous transgenes. In preferred embodiments, the heterologous transgene encodes a polypeptide or protein. The protein encoded within the viral genome may be any one of the protein cargos according to the invention allowing the viral cargo to act as a gene replacement therapy.

In some embodiments, the cargo-loaded albumin EV may additionally comprise one or more molecules that provide immune effector functions. Immune effector molecules are particularly useful in the case of albumin EVs loaded with a viral (e.g., AVV or lentiviral) cargo, but may equally be used where the albumin EV is loaded with any cargo according to the invention. The immune effector may act to reduce immunogenicity of the albumin EV. In some embodiments, the immune effector functions stimulate immune inhibitors. In other embodiments, the immune effector functions inhibit immune stimulating molecules. In some embodiments, albumin-EV comprises molecules that stimulate immune inhibitors and molecules that inhibit immune stimulating molecules.

Exemplary immune effector molecules include, but are not limited to, one or more of CTLA4, B7-1, B7-2, PD-I, PD-L1, PD-L2, CD28, or VISTA. In some embodiments, the EV further comprises CTLA4 and PD-L1, CTLA and PD-L2, CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-LI and VISTA.

The immune effector molecule may form part of the albumin EV fusion construct, the cargo, the targeting moiety or may form part of an entirely separate fusion protein construct comprising an immune effector molecule fused to any EV protein according to the present invention.

The present invention also relates to albumin EVs which are loaded with small molecule cargos. Although many small molecules exhibit good oral bioavailability, many small molecule drugs and biologics need to be given intravenously or via some other route of administration, be it for pharmacokinetic, pharmacodynamic, toxicity and/or stability reasons. Examples of small molecules include anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or non-steroidal anti-inflammatory drugs (NSAIDs) such as naproxen, aspirin, or celecoxib, antibiotics such as heracillin, or antihypertensives such as angiotensin-converting enzyme (ACE) inhibitors such as enalapril, ARBs such as candesartan, etc. The present invention is naturally applicable also to other small molecules without departing from the gist of the invention, as would be clear to a person skilled in the art.

In certain embodiments, the therapeutic cargo carried by the albumin EVs may be present on the inside of the EV, on the outside of the EV or in the membrane of the EV. The desired location of the therapeutic cargo will depend on the nature of the cargo and its mechanism of action, for instance, a membrane protein will preferably be located in the membrane of the EV, a decoy receptor will preferably be present on the surface of the EV, but a cargo designed to be delivered into the cytosol or nucleus of the recipient cell, such as a silencing RNA, will preferably be located inside the lumen of the EV.

The therapeutic cargo may be loaded passively into the EVs by the therapeutic cargo being present in the cytosol of the EV producing cells. Such passive loading applies, for instance, to nucleic acids, small molecules, viruses, soluble proteins or membrane proteins that are naturally loaded into the EVs.

In certain embodiments, the therapeutic cargo is actively loaded into the albumin EVs of the present invention. One form of active loading of cargos involves exogenous active loading, which involves cargo being loaded using any known exogenous loading method including: electroporation, transfection with transfection reagents such as cationic transfection agents, Lipofectamine®, conjugation of the cargo to a membrane-anchoring moiety, such as a lipid or cholesterol tail or tag, or loading by means of a CPP, either in the form of a CPP-cargo conjugate or in the form of a CPP-cargo non-covalent complex. Again, this type of active loading may result in the therapeutic cargo protein being located on the inside of the EV, on the outside of the EV or located within the membrane of the EV.

The therapeutic cargo may alternatively be loaded by another form of active loading into the albumin EVs of the present invention through use of fusion proteins.

This process of actively loading is often referred to as endogenous loading and has several benefits, significantly it allows the natural incorporation of the cargo into the EV without any need for conjugation steps after the EV is purified and ensures the EV retains all the benefits of being naturally derived such as retaining the corona proteins and natural morphology.

In this case, the therapeutic cargo carried by the EV forms part of the EV protein-albumin fusion protein or alternatively the therapeutic cargo carried by the EV forms part of an additional fusion protein with an EV-protein separate to the EV-protein-albumin fusion protein. In either case the therapeutic cargo protein may be fused into the fusion protein such that it is located on the inside of the EV, on the outside of the EV or located within the membrane of the EV. The presence of the EV protein in the fusion protein actively loads the therapeutic protein into the EV. The therapeutic cargo protein may be engineered to be fused to a single or multi-pass transmembrane protein at either the C or N terminus, to display the therapeutic protein on the surface of the EV or protect the therapeutic cargo within the EV. Any EV protein, as defined above, may be employed as a fusion partner for loading therapeutic protein cargos. EV proteins which may be employed to load cargo proteins into EVs may be transmembrane but need not be. Where the therapeutic cargo forms part of an additional fusion protein with an EV-protein separate to the EV-protein-albumin fusion protein. In this embodiment the fusion protein may include an EV protein which is membrane associated, rather than transmembrane. For instance, the fusion protein may employ EV proteins, which associate with the luminal/intravesicular surface of the EV membrane, ensuring that the therapeutic cargo is loaded into the lumen of the EV. Alternatively, the cargo may be fused to an EV protein, which associates with the outer surface of the EV membrane. Particularly advantageous EV proteins include CD63, CD81, CD9, CD82, CD44, CD47, CD55, LAMP2B, ICAMs, integrins, ARRDC1, syndecan, syntenin, and Alix, as well as derivatives, domains, variants, mutants, or regions thereof.

The therapeutic protein may also be engineered into an extravesicular loop or loops of a multi-pass transmembrane protein, optionally wherein the multi-pass transmembrane protein is a tetraspanin, such as CD36, CD9, CD81 or any of TSPAN1-TSPAN33. Advantageously, cargo proteins may be incorporated into more than one of the loops of said multi-pass transmembrane protein and/or more than one cargo protein may be incorporated into each loop of said multi-pass transmembrane protein. More than one cargo protein may be incorporated into the transmembrane fusion protein without affecting the expression of the transmembrane protein as the transmembrane protein is still able to correctly fold and form, meaning that the transmembrane protein is more stable and thus the cargo is more likely to remain stably introduced into the EV protein. The cargo may be in the same fusion protein as the albumin (aka the albumin fusion protein). Alternatively, the cargo may be incorporated into a second fusion protein separate the albumin fusion protein.

Without wishing to be bound by theory, the presence of more than one therapeutic cargo protein per transmembrane EV protein means that more cargo molecules can be bound to the EV per fusion protein expressed, which can increase the therapeutic potency of the EV and allows for different therapeutic cargos to be incorporated into the same EV, significantly increasing the versatility of the EVs produced.

In embodiments where the therapeutic cargo is actively loaded (i.e., endogenously loaded) into the EVs utilizing a separate fusion protein construct, i.e., on an additional fusion construct, which does not comprise the albumin, said additional fusion construct may also further comprise: (i) at least one multimerization domain; (ii) at least one endosomal escape domain; (iii) at least one linker/spacer/scaffold sequence; (iv) at least one release domain or releasable linker capable of cleavage to release the therapeutic cargo; (v) at least one immune effector molecule; and/or (vi) at least one targeting moiety.

Suitable release domains according to the present invention include, but are not limited to, cis-cleaving sequences such as inteins, light induced monomeric or dimeric release domains such as Kaede, KikGR, EosFP, tdEosFP, mEos2, PSmOrange, the GFP-like Dendra proteins, Dendra and Dendra2, CRY2-CIBN, etc. Alternatively, nuclear localization signal (NLS)-nuclear localization signal-binding protein (NLSBP) (NLS-NLSBP) release system may be employed. Protease cleavage sites may also be incorporated into the fusion proteins for spontaneous release etc., depending on the desired functionality of the fusion polypeptide. In the case of nucleic acid cargos, specific nucleic acid cleaving domains may be included. Non-limiting examples of nucleic acid cleaving domains include, but are not limited to, endonucleases such as Cas6, Cas13, engineered PUF nucleases, site specific RNA nucleases etc.

Without wishing to be bound by theory, the inclusion of release domains can enable release of particular parts or domains from the original fusion polypeptide. This is particularly advantageous when the release of parts of the fusion polypeptide would increase bioactive delivery of the cargo and/or when a particular function of the fusion polypeptide works better when part of a smaller construct.

The EVs as per the present invention may comprise at least one targeting moiety. Targeting moieties may be proteins, peptides, single chain fragments or any other derivatives of antibodies, obtainable from either humans or from non-human animals etc. The targeting moiety may form part of the albumin EV fusion construct or alternatively it may form part of a separate polypeptide construct which is comprised in the EV. EVs comprising targeting moieties may be produced using any of the methods disclosed herein.

The presence of the targeting moiety, combined with the improved biodistribution of albumin-display EVs, makes them especially useful in organ targeting. Without wishing to be bound by theory, the albumin-display EV can remain in circulation much longer, avoiding uptake by the liver or cells of the immune systems, and thus is able to reach the desired target organ.

The targeting moiety may be fused to any EV protein according to the invention. The targeting moiety-EV protein fusion may be engineered to display the targeting moiety on the surface of the EV by fusion to the extravesicular portion of a single pass transmembrane protein. Alternatively, the targeting moiety may be engineered into an extravesicular loop or loops of a multi-pass transmembrane protein, optionally wherein the multi-pass transmembrane protein is a tetraspanin, such as CD36, CD9, CD81 or any of TSPAN1-TSPAN33.

Where the targeting moiety is present on a separate fusion protein construct, i.e., on an additional fusion construct which does not comprise albumin, said additional fusion construct may also further comprise: (i) at least one multimerization domain; (ii) at least one endosomal escape domain; (iii) at least one immune effector molecule; and/or (iv) at least one linker/spacer/scaffold sequence.

Targeting moieties may be used to target the EVs to cell, subcellular locations, tissues, organs or other bodily compartments. Organs and cell types that may be targeted include, but are not limited to, the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells.

As would be appreciated by the skilled person, targeting can be achieved by a variety of means, for instance, the use of targeting peptides. Such targeting peptides may be anywhere from a few amino acids in length to several 100s of amino acids in length, e.g., anywhere in the interval of about 3-200, 3-150, 3-100 amino acids, about 50-175, 50-125, 50-75, 3-30 amino acids, about 5-25 amino acids, e.g., about 7 amino acids, about 12 amino acids, about 20 amino acids, etc. Targeting peptides of the present invention may also include full length proteins, such as receptors, receptor ligands, etc. Furthermore, the targeting peptides as per the present invention may also include antibodies and antibody derivatives, e.g., monoclonal antibodies, scFvs, other antibody domains, such as nanobodies, etc. Exemplary targeting moieties include brain targeting moieties such as rabies virus glycoprotein (RVG), nerve growth factor (NGF), melanotransferrin and the FC5 Peptide and muscle targeting moieties such as Muscle Specific Peptide (MSP).

Populations of EVs in accordance with the present invention are also envisaged, wherein the EV population comprises at least one albumin present on the surface of the EV. In certain embodiments, the average number of albumins per EV in the population of EVs according to the invention is above one albumin per EV, but it may also be below one albumin per EV. Furthermore, in certain embodiments, in the population of EVs according to the invention, the average number of cargo molecules per EV is above or below one cargo molecule per EV. In another embodiment, in the population of EVs according to the invention, at least 5%, at least 10%, at least 20%, at least 50%, at least 70%, at least 75%, at least 80%, at least 85%, at least 90%, and/or at least 95% of all EVs comprise at least one albumin and optionally also at least one cargo molecule.

The present invention also relates to fusion proteins comprising at least one albumin and at least one EV protein, and polynucleotide constructs encoding such fusion proteins, as well as vectors, EVs and cells comprising such constructs.

The EV protein may optionally be a transmembrane EV protein or an EV protein associated with the outer membrane of the EV. Where the EV protein is a multi-pass transmembrane EV protein, the albumin may be engineered into an extravesicular loop or loops of the multi-pass transmembrane protein. In preferred embodiments, the multi-pass transmembrane EV protein of the fusion protein is a tetraspanin. The fusion proteins of the invention may advantageously comprise more than one albumin i.e., a plurality of albumins. The plurality of albumins may the same or different to one another.

Fusion proteins according to the present invention may further comprise: (i) at least one multimerization domain; (ii) at least one endosomal escape domain; (iii) at least one linker/spacer/scaffold sequence; (iv) at least one therapeutic cargo protein, optionally further comprising a release domain or releasable linker capable of cleavage to release the therapeutic cargo; (v) at least one immune effector molecule; and/or (vi) at least one targeting moiety.

For clarity, the structures of a few albumin-EV protein fusion proteins are illustrated below:
EV protein-albumin
EV protein domain I-albumin-EV protein domain II
EV protein domain I-albumin-albumin-EV protein domain II
EV protein domain I-albumin-EV protein domain II-albumin-EV protein domain III
EV protein domain I-albumin-EV protein domain II-therapeutic cargo
Therapeutic cargo-EV protein domain I-albumin-EV protein domain II
EV protein domain I-albumin-EV protein domain II-targeting moiety

The present invention relates to EVs comprising the above identified fusion proteins.

Generally, EVs may be derived from essentially any cell source, be it a primary cell source or an immortalized cell line. The EV source cells may be any embryonic, fetal, or adult somatic stem cell types, including induced pluripotent stem cells (iPSCs) and other stem cells derived by any method, as well as any adult cell source. The source cells per the present invention may be selected from a wide range of cells and cell lines, for instance mesenchymal stem or stromal cells (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial (AE) cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, mesenchymal stromal cells (MSCs) of different origin, amnion cells, AE cells, CEVEC's CAP® cells, any cells obtained through amniocentesis or from the placenta, airway or alveolar epithelial cells, fibroblasts, endothelial cells, etc. Also, immune cells, such as B cells, T cells, NK cells, macrophages, monocytes, dendritic cells (DCs) are also within the scope of the present invention, and essentially any type of cell which is capable of producing EVs is also encompassed herein. The source cell may be either allogeneic, autologous, or even xenogeneic in nature to the patient to be treated, i.e., the cells may be from the patient him/herself or from an unrelated, matched or unmatched donor.

Particularly, useful are cells which have been stably modified to comprise at least one monocistronic, bicistronic or multicistronic polynucleotide construct according to the invention as hereinbefore defined encoding a fusion protein of an EV protein and at least one albumin protein. Such cells may be stably or transiently transfected with the polynucleotides according to the present invention to render them albumin-EV producing cells. Such cells may also be stably or transiently modified to include a construct encoding for a therapeutic cargo protein which optionally may form part of a fusion protein with an EV protein. Such cells may also be stably or transiently modified to include a construct encoding for a targeting moiety which comprises a fusion protein of the targeting moiety and an EV protein. The cells of the present invention may be of a monoclonal cell or a polyclonal cell line.

Preferred producer cells according to the present invention may include, but are not limited to, a HEK cell, a HEK293 cell, a HEK293T cell, an MSC, in particular a WJ-MSC cell or a BM-MSC cell, a fibroblast, an amnion cell, an AE cell, CEVEC's CAP® cells, a placenta-derived cell, a cord blood cell, an immune system cell, an endothelial cell, an epithelial cell or any other cell type, wherein said cells may be, for instance, adherent cells, suspension cells, and/or suspension-adapted cells.

The present invention relates to a method for producing the EVs according to the invention. The method for producing EVs comprises:
(i) introducing into an EV-producing cell at least one polynucleotide fusion construct encoding an albumin-EV protein fusion protein; and
(ii) expressing said construct in the EV-producing cell, thereby generating an EV comprising albumin protein present on the surface of the EV.

The method for producing the EVs may further comprise a step of loading the EV with at least one cargo molecule. Said cargo loading step may be by endogenous loading or exogenous loading. Where the cargo is loaded by endogenous means the cargo is either loaded by the same fusion construct as the albumin protein or the cargo is loaded by a second fusion construct that encodes for a Cargo-EV fusion protein.

Where the cargo loading step is an exogenous loading step, the loading step may comprise: loading of the cargo by any exogenous loading method including electroporation, microfluidics, transfection with transfection reagents such a cationic transfection agents, Lipofectamine®, conjugation of the cargo to a membrane anchoring moiety such as a lipid or cholesterol tail or tag or loading by means of a CPP, either in the form of a CPP-cargo conjugate or in the form of a CPP-cargo non-covalent complex.

Where the cargo loading step is endogenous, the endogenous loading step may comprise introducing the cargo protein into the albumin EV protein fusion construct or introducing into the EV-producing cell a further nucleic acid construct encoding the therapeutic cargo. It is also possible that a single nucleic construct might encode separately both the albumin-EV protein fusion protein as well as the cargo protein using a bidirectional plasmid. Said therapeutic cargo construct may simply be expressed by the EV-producing cell and passively loaded into the EVs or the therapeutic cargo may be comprised as a fusion protein with an EV protein, so that the cargo protein, when translated, is endogenously and actively loaded into the EVs produced by the EV-producing cell.

The method for producing the EVs may further comprise a step of loading the EV with at least one targeting moiety. In this embodiment, the targeting moiety loading step is an endogenous loading step and may comprise introducing into the EV-producing cell a further nucleic acid construct encoding the targeting moiety, which is comprised as a fusion protein with an EV protein, so that, when translated, the targeting moiety is endogenously and actively loaded into the EVs produced by the EV-producing cell.

Without wishing to be bound by theory, the benefit of generating the cells as double or multiple stable cells is that a large library of producer cell lines can be generated quickly and easily by swapping out the cargo and/or targeting construct. Additionally, each separate construct can be placed under the control of a different promoter and thus the expression levels of the albumin, cargo and/or targeting moiety can be carefully and individually controlled. Alternatively, when the therapeutic cargo and/or the targeting moiety forms part of the albumin-EV protein fusion construct, the advantage of a single albumin-EV protein-cargo/targeting-moiety construct is that it only requires the cells to be made single stable, which results in the generation of simple and robust cell lines. The choice of a single, double, or multiple stable cell line will depend upon the cargo and targeting moieties desired, their size and desired location on the EV.

Purification of EVs is achieved by any method including, but not limited to, techniques comprising liquid chromatography (LC), high-performance liquid chromatography (HPLC), bead-eluate chromatography, ionic exchange chromatography, spin filtration, tangential flow filtration (TFF), hollow fiber filtration, centrifugation, immunoprecipitation, flow field fractionation, dialysis, microfluidic-based separation, etc., or any combination thereof. In an advantageous embodiment, the purification of the EVs is carried out using a sequential combination of filtration (preferably ultrafiltration (UF), TFF or hollow fibre filtration) and affinity chromatography, optionally also including size exclusion LC or bead-eluate LC. Combining purification steps normally enhances the purity of the resulting samples and, in turn, leads to superior therapeutic activity. Further, as compared to ultracentrifugation (UC), which is routinely employed for purifying exosomes, sequential filtration-chromatography is considerably faster and possible to scale to higher manufacturing volumes, which is a significant drawback of the current UC methodology that dominates the prior art. Another advantageous purification method is TFF, which offers scalability and purity, and which may be combined with any other type of purification technique.

The EVs of the present invention, in some embodiments, are isolated EVs. Accordingly, the present invention provides isolated EVs comprising at least one albumin protein present on the surface of the EV, wherein the albumin protein forms part of a fusion protein with an EV protein.

The present invention also relates to pharmaceutical compositions comprising at least one EV according to the invention and a pharmaceutically acceptable excipient, diluent, vehicle, solvent or carrier.

Exemplary excipients include degradation or loss of activity stabiliser excipients including proteins such as HSA, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca2+, Zn2+ and EDTA, cyclodextrin-based excipients such as hydroxypropyl ß-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol.

The EVs as per the present invention may be administered to a human or animal subject via various different administration routes, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the cargo molecule in question, or the EV population as such.

It will be clear to the skilled artisan that, when describing medical and scientific uses and applications of the EVs, the present invention normally relates to a plurality of EVs, i.e., a population of EVs which may comprise thousands, millions, billions or even trillions of EVs. EVs may be present in concentrations such as about 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume or per unit of weight (for instance per ml or per L or per kg of body weight), or any other population number that is larger, smaller, or anywhere in between. In the same vein, the term “population”, which may e.g., relate to an EV or exosome comprising a certain albumin or cargo, shall be understood to encompass a plurality of entities constituting such a population. In other words, individual EVs or exosomes, when present in a plurality, constitute a population. Thus, naturally, the present invention pertains both to individual EVs or exosomes and populations comprising EVs or exosomes, as will be clear to the skilled person. The dosages of EVs when applied in vivo may naturally vary considerably depending on the disease to be treated, the administration route, the activity and/or effects of the cargo of interest, the albumin protein, any targeting moieties present on the EVs, the pharmaceutical formulation, etc.

It is envisaged that any dosage regime would be applicable to the albumin EVs of the invention. The dosage regime chosen will depend on the cargo being delivered by the albumin EVs and the disease to be treated and any additional therapies being administered which will be determined by the skilled physician. The EV (or exosome) may be modified to display a select fragment and/or number of albumin proteins adaptable to the cargo, such that the longevity of the EV becomes more predictable. Meaning that the frequency and schedule of repeat dosages may be modified and thus side-effects avoided and/or reduced.

It is envisaged that the albumin EVs of the present invention will be administered multiple times, i.e., more than once, but normally more than two times or potentially for chronic, long-term treatment (i.e., administered tens to hundreds to thousands of times). Preferably, if the cargo is an antigen that is being administered as a vaccine, the immunization schedule will involve two or more administrations of the polypeptide, spread out over several weeks. Similarly, if the cargo is e.g., an RNA agent such as an siRNA or mRNA or a protein such as an antibody or an enzyme or a transporter, or a viral cargo such as an AAV or lentivirus, the albumin EVs comprising the cargo in question will likely be administered more than once, normally multiple times as part of a chronic treatment regimen.

The present invention also relates to EVs according to the invention for use in medicine. The present invention also relates to a pharmaceutical composition according to the invention for use in medicine. The present invention also relates to a method of treatment comprising administering to a patient in need thereof, at least one effective amount of the EVs according to the invention or at least one effective amount of a pharmaceutical composition of the invention. The present invention also relates to a method of treating at least one disease, disorder and/or condition in a patient, the method comprising administering to the patient at least one effective amount of the EVs according to the invention or at least one effective amount of a pharmaceutical composition of the invention.

The medical use or method of treatment may be by delivery of any kind of cargo according to the invention. For instance, the medical use or treatment may be by delivery of functional proteins as protein replacement therapy, delivery of mRNA encoding functional proteins to also act as a protein replacement therapy. Such a protein replacement therapy may, for instance, be ERT for diseases caused by inborn errors in metabolism, such as phenylketonuria (PKU), urea cycle disorders, or lysosomal storage disorders. The medical use or treatment may be by delivery of gene silencing RNAs, splice switching RNAs, or CRISPR-Cas9 for gene editing. The medical use or treatment may be gene therapy by delivery of plasmid DNA, mini-circles or viral gene therapies such as AAVs or lentiviruses. The medical use or treatment may be by presentation of an antigen or neoantigen for immunotherapy, in effect acting as a vaccine to induce an immune response. For instance, the EV may act by delivery and/or presentation of a tumour antigen for cancer immunotherapy, or viral, bacterial or fungal antigens for immunization against pathogens. The medical use or treatment may be by delivery of small molecules, biologics, antibodies or antibody-drug conjugates capable of mediating a therapeutic effect once delivered into a cell or the extracellular matrix. In one embodiment, the medical use or treatment may be affected by the EVs comprising more than one type of therapeutic cargo, i.e., the therapeutic cargo may be a mixture of protein, nucleic acid, virus, viral genome, antigen, small molecule and/or biologic.

Importantly, the present invention relates to use of the EVs or pharmaceutical composition described herein in the prophylaxis and/or treatment and/or alleviation of a variety of diseases, typically via the delivery of essentially any type of drug cargo, such as for instance: a nucleic acid such as an RNA molecule, a DNA molecule or a mixmer, mRNA, antisense or splice-switching oligonucleotides, siRNA, shRNA, miRNA, pDNA, supercoiled or unsupercoiled plasmids, mini-circles, peptides or proteins including transporters, enzymes, receptors such as decoy receptors, membrane proteins, cytokines, antigens and neoantigens, ribonuclear proteins, nucleic acid binding proteins, antibodies, nanobodies, antibody fragments, antibody-drug conjugates, small molecule drugs, gene editing technology such as CRISPR-Cas9, TALENs, meganucleases, or vesicle-based cargos such as viruses (e.g. AAVs, lentiviruses, etc.). The cargo may be a mixture of protein, nucleic acid, virus, viral genome, antigen, small molecule and/or biologic.

Non-limiting examples of diseases and conditions that are suitable targets for treatment using the EVs and pharmaceutical compositions described herein include the following non-limiting examples: autoimmune diseases (such as celiac disease, Crohn's disease, diabetes mellitus type 1, Graves' disease, inflammatory bowel disease, multiple sclerosis, psoriasis, rheumatoid arthritis, systemic lupus erythematosus, ulcerative colitis, ankylosing spondylitis, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis), stroke, acute spinal cord injury, vasculitis, Guillain-Barré syndrome, acute myocardial infarction, acute respiratory distress syndrome (ARDS), sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, haemophilia type A, B or C, Duchenne muscular dystrophy (DMD) and other muscular dystrophies, in-born errors of metabolism including disorders of carbohydrate metabolism, e.g., G6PD deficiency galactosemia, hereditary fructose intolerance, fructose 1,6-diphosphatase deficiency and the glycogen storage diseases, disorders of organic acid metabolism (organic acidurias), such as alkaptonuria, 2-hydroxyglutaric acidurias, methylmalonic or propionic acidemia, multiple carboxylase deficiency, disorders of amino acid metabolism, such as PKU, maple syrup urine disease, glutaric acidemia type 1, aminoacidopathies, e.g., hereditary tyrosinemia, nonketotic hyperglycinemia, and homocystinuria, hereditary tyrosinemia, Fanconi syndrome, Primary Lactic Acidoses e.g., pyruvate dehydrogenase, pyruvate carboxylase and cytochrome oxidase deficiencies, disorders of fatty acid oxidation and mitochondrial metabolism, such as short, medium, and long-chain acyl-CoA dehydrogenase deficiencies also known as Beta-oxidation defects, Reye's syndrome, medium-chain acyl-coenzyme A dehydrogenase deficiency (MCADD), mitochondrial encephalopathy lactic acidosis and stroke-like episodes (MELAS), myoclonic epilepsy with ragged red fibers (MERRF), pyruvate dehydrogenase deficiency, disorders of porphyrin metabolism such as acute intermittent porphyria, disorders of purine or pyrimidine metabolism such as Lesch-Nyhan syndrome, disorders of steroid metabolism such as lipoid congenital adrenal hyperplasia, congenital adrenal hyperplasia, disorders of mitochondrial function such as Kearns-Sayre syndrome, disorders of peroxisomal function such as Zellweger syndrome and neonatal adrenoleukodystrophy, congenital adrenal hyperplasia or Smith-Lemli-Opitz, Menkes syndrome, neonatal hemochromatosis, urea cycle disorders such as N-acetylglutamate synthase deficiency, carbamoyl phosphate synthetase deficiency, OTC deficiency, citrullinemia (deficiency of argininosuccinic acid synthase), argininosuccinic aciduria (ASA; deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1), orotic aciduria (deficiency in the enzyme, UMPS), all of the lysosomal storage diseases, for instance alpha-mannosidosis, beta-mannosidosis, aspartylglucosaminuria, cholesteryl ester storage disease, cystinosis, Danon disease, Fabry disease, Farber disease, fucosidosis, galactosialidosis, Gaucher disease Type I, Gaucher disease Type II, Gaucher disease Type III, GM1 gangliosidosis Type I, GM1 gangliosidosis Type II, GM1 gangliosidosis Type III, GM2—Sandhoff disease, GM2—Tay-Sachs disease, GM2—gangliosidosis, AB variant, mucolipidosis II, Krabbe disease, lysosomal acid lipase deficiency, metachromatic leukodystrophy, MPS I—Hurler syndrome, MPS I—Scheie syndrome, MPS I—Hurler-Scheie syndrome, MPS II—Hunter syndrome, MPS IIIA—Sanfilippo syndrome Type A, MPS IIIB—Sanfilippo syndrome Type B, MPS IIIB—Sanfilippo syndrome Type C, MPS 111B—Sanfilippo syndrome Type D, MPS IV Morquio Type A, MPS IV—Morquio Type B, MPS IX—hyaluronidase deficiency, MPS VI—Maroteaux-Lamy, MPS VII—Sly syndrome, Mucolipidosis I—sialidosis, mucolipidosis IIIC, mucolipidosis Type IV, mucopolysaccharidosis, multiple sulfatase deficiency, neuronal ceroid lipofuscinosis T1, neuronal ceroid lipofuscinosis T2, neuronal ceroid lipofuscinosis T3, neuronal ceroid lipofuscinosis T4, neuronal ceroid lipofuscinosis T5, neuronal ceroid lipofuscinosis T6, neuronal ceroid lipofuscinosis T7, neuronal ceroid lipofuscinosis T8, neuronal ceroid lipofuscinosis T9, neuronal ceroid lipofuscinosis T10, Niemann-Pick disease Type A, Niemann-Pick disease Type B, Niemann-Pick disease Type C (NPC), Pompe disease, pycnodysostosis, Salla disease, Schindler disease and Wolman disease, etc., cystic fibrosis, primary ciliary dyskinesia, pulmonary alveolar proteinosis, arthrogryposis-renal dysfunction-cholestasis (ARC) syndrome, Rett syndrome, neurodegenerative diseases including Alzheimer's disease, Parkinson's disease, GBA associated Parkinson's disease, Huntington's disease and other trinucleotide repeat-related diseases, prion diseases, dementia including frontotemporal lobe dementia, amyotrophic lateral sclerosis (ALS), motor neuron disease, multiple sclerosis, cancer-induced cachexia, anorexia, diabetes mellitus type 2, and various cancers.

The present invention provides EVs comprising at least one albumin, wherein the EVs accumulate within a tumor and/or the lymph nodes at a level greater than the level of accumulation exhibited by otherwise identical EVs that lack the at least one albumin. Specifically, the level of tumor and/or lymph node accumulation as compared to EVs lacking albumin may be at least 2-fold, or at least 3-fold, or at least 4-fold, or at least 5-fold, or at least 6-fold, or at least 7-fold or at least 8-fold, or at least 9-fold, or at least 10-fold, or at least 20-fold or more.

The present invention is advantageous for the treatment of cancers; and especially advantageous, in the treatment of cancer by immunotherapy, due to accumulation in the lymph nodes.

Specifically, it is envisaged that the present invention is useful in the treatment of cancer by immunotherapy, i.e., the presentation of cancer antigens on the surface of albumin EVs so that those antigens raise an immune response against the cancer antigen. Virtually all types of cancer are relevant disease targets for the present invention, for instance, acute lymphoblastic leukemia (ALL), acute myeloid leukemia, adrenocortical carcinoma, AIDS-related cancers, AIDS-related lymphoma, anal cancer, appendix cancer, astrocytoma, cerebellar or cerebral, basal-cell carcinoma, bile duct cancer, bladder cancer, bone tumor, brainstem glioma, brain cancer, brain tumor (cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumor (childhood, gastrointestinal), carcinoma of unknown primary, central nervous system lymphoma, cerebellar astrocytoma/malignant glioma, cervical cancer, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, extracranial germ cell tumor, extragonadal germ cell tumor, extrahepatic bile duct cancer, eye Cancer (intraocular melanoma, retinoblastoma), gallbladder cancer, gastric (stomach) cancer, gastrointestinal carcinoid tumor, gastrointestinal stromal tumor (GIST), germ cell tumor (extracranial, extragonadal, or ovarian), gestational trophoblastic tumor, glioma (glioma of the brain stem, cerebral astrocytoma, visual pathway and hypothalamic glioma), gastric carcinoid, hairy cell leukemia, head and neck cancer, heart cancer, hepatocellular (liver) cancer, Hodgkin lymphoma, hypopharyngeal cancer, intraocular melanoma, islet cell carcinoma (endocrine pancreas), Kaposi sarcoma, kidney cancer (renal cell cancer), laryngeal cancer, leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia)), lip and oral cavity cancer, liposarcoma, liver cancer (primary), lung cancer (non-small cell, small cell), lymphomas, AIDS-related lymphoma, Burkitt lymphoma, cutaneous T-cell lymphoma, Hodgkin lymphoma, non-Hodgkin, medulloblastoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic/myeloproliferative diseases, yelogenous leukemia, chronic myeloid leukemia (acute, chronic), myeloma, nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic islet cell cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, pleuropulmonary blastoma, prostate cancer, rectal cancer, renal cell carcinoma (kidney cancer), retinoblastoma, rhabdomyosarcoma, salivary gland cancer, sarcoma (Ewing family of tumors sarcoma, Kaposi sarcoma, soft tissue sarcoma, uterine sarcoma), Sézary syndrome, skin cancer (nonmelanoma, melanoma), small intestine cancer, squamous cell, squamous neck cancer, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine cancer, uterine sarcoma, vaginal cancer, vulvar cancer, Waldenström macroglobulinemia, and/or Wilm's tumor.

The present invention is also specifically advantageous for the treatment of brain and CNS disorders due to the increased biodistribution of albumin EVs to the brain. The present invention provides EVs comprising at least one albumin, wherein the EVs accumulate within the brain at a level greater than the level of accumulation exhibited by otherwise identical EVs that lack the at least one albumin. Specifically, the level of tumor accumulation as compared to EVs lacking albumin may be at least 2-fold, or at least 3-fold, or at least 4-fold, or at least 5-fold, or at least 6-fold, or at least 7-fold or at least 8-fold, or at least 9-fold, or at least 10-fold, or at least 20-fold or more. The present invention specifically relates to EVs with albumin present on the surface of the EV, which are loaded with any type of cargo according to the invention, but preferably a nucleic acid cargo, such as silencing RNAs, such as siRNAs, which target RNAs known to be involved with neurodegenerative diseases such as Alzheimer's disease, Parkinson's disease, Huntington's disease, spinocerebellar ataxia, ALS, frontal temporal dementia, motor neuron disease, multiple sclerosis, Wallerian degeneration and bullous retinoschisis, Goldberg-Shprintzen syndrome, kuru, autoimmune GFAP astrocytopathy, methyl CpG binding protein 2 (MECP2) duplication syndrome, aquaporin-4 (AQP4)-astrocytopathy, familial pain syndromes such as erythromelalgia, paroxysmal extreme pain disorder and congenital insensitivity to pain, Pelizaeus-Merzbacher disease, prion diseases including Creutzfeldt-Jakob disease (CJD), Gerstmann-Sträussler-Scheinker syndrome (GSS), and fatal familial insomnia (FFI), leukodystrophies including demyelinating, adult-onset, autosomal dominant and leukodystrophy.

The present invention specifically relates to EVs with albumin present on the surface, which are further loaded with any therapeutic cargo according to the invention, the therapeutic cargo may be both protein and/or nucleic acid cargos, for the treatment of DMD, diseases caused by in-born errors of metabolism including antisynthetase syndrome (ASSd), lysosomal storage disorders including NPC and Pompe disease, urea cycle disorders such as ASA and citrullinemia and OTC deficiency, metachromatic leukodystrophy and PKU.

Increasing the shelf-life of complex products, such as therapeutic exosomes, is vitally important to enabling the enormous therapeutic benefits of EVs to be realised. Furthermore, EVs are known to be structurally susceptible to damage due to the exposure of vulnerable phosphatidylserine to repeated freeze-thaw cycles. The present invention aims to overcome these problems.

The present invention also relates to a method of producing EVs with increased storage stability and/or shelf-life, wherein the method comprises: a) obtaining EVs according to the invention, and b) formulating said EVs in a storage or formulation buffer which comprises albumin. Any form of albumin may be utilised, optionally the albumin is recombinant albumin, human albumin, serum albumin, HSA, recombinant serum albumin, recombinant HSA, Albunorm® (a human-serum derived albumin solution) or any fragment or domain therefore that binds to the albumin of the invention. HSA has a particularly long half-life, so is preferred. This fact also means that the half-life extensions observed in the mouse data in this application can be expected to be improved still further when the albumin-EVs of the present invention are tested in humans.

Without wishing to be bound by theory, the presence of albumin on the surface of the EVs has the advantage of creating a protective shield around the EVs. This possibly prevents aggregation of EVs, but also encourages the formation of larger heterogenous nanoparticles, which protect the EVs from damaging freeze-thaw cycles and shear stress during processing. This results in a much more robust EV population with a long shelf life. Without wishing to be bound by theory, it is believed that the albumin coat prevents unwanted interaction of the EV membrane with the walls of the containers, meaning that more EVs are retrieved after storage and additionally those that are retrieved are of high quality.

The additional benefit of using albumin directly displayed on the EV for extending the shelf life of these compositions is that the albumin-display EVs can be directly administered to patients and the albumin protein will then function to increase the half-life of the EV in circulation. Thus, by only one modification to the EVs, (i.e., the inclusion of albumin), the EVs are made more robust during both storage and formulation. This means they have not only greater therapeutic efficacy but loss of EVs due to adherence to container surfaces is reduced and, importantly, when those EVs are then administered to a patient, they also have increased circulation time in vivo.

The present invention provides EVs comprising at least one albumin, wherein the EVs exhibit a shelf-life that is at least 5%, at least 10%, at least 20%, at least 30%, at least 50%, at least 70%, at least 90% or more than the shelf-life exhibited by otherwise identical EVs that lack at the least one albumin. The EVs may exhibit a shelf-life that is weeks, months, or years greater than the shelf-life exhibited by otherwise identical EVs that lack at least one albumin, for instance, the shelf-life may be increased by 5 weeks, 10 weeks, 20 weeks, 1 month, 3 months, 6 months, 9 months, 1 year, 2 years or more.

It is important to note that albumin does not contain blood-derived impurities that otherwise could activate unwanted cell pathways and result in a product which is unable to meet regulatory approval requirements regarding host cell protein levels. Recombinant albumin can help maintain the safety and efficacy of final drug products during storage by avoiding the presence of host cell proteins.

Displaying albumin protein on the surface of EVs, such as recombinant albumin, has the additional benefit that it can be administered to a patient and there be no delay in said genetically modified EV acquiring extension of half-life post administration. This is due to the genetically modified EV already displaying albumin protein on the surface of the EV which acts as a protective layer to prevent elimination and excretion of the EV from the patient. Such albumin-display EVs will also exhibit significantly reduced uptake of the genetically modified EVs at the injection site (i.e., a reduction in off-target uptake). This, again, results in more of the administered dose of albumin-display EVs being immediately present in circulation and for longer, thus increasing incidence rate of the albumin-display EVs localising at a desired site of action, engaging and/or binding a desired target tissue/cell etc., and in turn increasing the therapeutic efficacy of the albumin-display EVs. Minimising off-target uptake also has the effect of reduced toxicity and undesirable side-effects associated with off-target uptake.

Where the albumin-display EV comprises a drug cargo an advantage of said albumin-display EVs is that the surface albumin provides a population of EVs that serves as a drug cargo reservoir.

Another advantage of albumin-display EVs is that, where the EV also comprises a drug cargo, the surface albumin competes with circulating albumin in the vasculature of a patient preventing the circulating albumin from binding to the EV and/or drug cargo contained therein. The effect being that a more predictable and stable PK/PD profile results due to lower incidence of secondary interaction with free circulating albumin present in the subject(s) microvasculature.

The present invention also relates to pharmaceutical compositions comprising the nanoparticle complexes of the invention combined with a pharmaceutically acceptable excipient or carrier.

In further aspects, the present invention thus also relates to EVs, EV-protein complexes, and/or pharmaceutical compositions comprising such EVs for use in medicine, preferably in the treatment of diseases which would benefit from antibody- or Fc domain-containing protein-based treatment, antibody-drug conjugate, and/or antibody-mediated targeting.

As described above, the albumin-display EVs has the additional benefit of a more immediate protective effect of albumin, due to the albumin being present on administration (i.e., at time 0). In turn, this increases the half-life and thus increases the therapeutic efficacy of the albumin-display EVs.

The present invention also pertains to affinity chromatography isolation and purification of the EVs of the invention, wherein the EVs are engineered to enable highly specific binding to e.g., chromatography matrices, and optionally subsequent elution. EVs according to the present invention comprise albumin proteins on the surface of the EV. Purification of said EVs by affinity purification is possible utilising affinity of the albumin present on the EVs for a corresponding binding partner, for example FcRN.

Conventional methods to prepare and isolate EVs (e.g., exosomes) involve a series of differential centrifugation steps to separate the vesicles from cells or cell debris present in the culture medium into which the EVs are released by EV-producing cells. Typically, series of centrifugations at e.g., 300 g, 10,000 g and 70,000 g or 100,000 g are applied, upon which the resulting pellet at the bottom of the tube is resuspended to a fraction of its original volume with a saline solution to constitute a concentrated EV or exosome solution. However, these methods are essentially unsuitable for clinical applications for a number of reasons: (1) the extended length of time needed for the entire process, (2) issues around scale-up and validation in a GMP environment, (3) significant risk of contamination by cell debris, resulting in host cell protein contamination at levels unacceptable for regulatory approval, (4) poor reproducibility due to operator variability, (5) aggregation of EVs/exosomes resulting from pelleting of the vesicles, (6) low recovery at end of processing, and (7) negative impact on vesicle morphology and thereby biodistribution and activity. There is therefore a need for improved methods of preparing membrane vesicles, suitable with industrial constraints and allowing production of vesicle preparations of therapeutic quality. To that end, International Patent Publication No. WO 2000/044389 discloses methods for preparing membrane vesicles from biological samples through chromatographic techniques, such as anion exchange chromatography and/or gel permeation chromatography. However, there is room for significant improvement over said disclosures, especially as the EV therapeutics field advances toward clinical translation and impact of EV-based therapies. The previously known methods for purifying exosomes are not ideally suited to large scale production and scale up that would be necessary for commercial production of EV therapeutics. The present invention allows much larger scale purification of engineered exosomes with high affinity than would be achievable with previously known methods.

The present invention achieves these, and other, objectives by utilizing chromatography matrices comprising albumin domains, which have affinity for the EVs of the present invention (which are engineered to comprise albumin binding polypeptides on the surface of the EVs). As such, the present invention thus relates to various aspects and embodiments surrounding processes for isolating and/or purifying EVs. Advantageously, the surface albumin protein is used to purify the EVs. Once the albumin-display-EVs are produced and purified, the albumin protein present on the surface endows the EVs with increased shelf-life and a prolonged half-life. Addition of albumin to the EVs has the benefit of multi-functionality of allowing not only purification but also improved half-life and shelf-life. The addition of albumin therefore generates extremely versatile EVs with only a single genetic engineering step.

The present invention provides EVs comprising at least one albumin, wherein the EVs exhibit a half-life in a human that is at least 5%, at least 10%, at least 20%, at least 30%, at least 50% or more than the half-life exhibited by otherwise identical EVs that lack at least one albumin. For instance, the EVs may exhibit a half-life in a human/animal that is at least 10 minutes, at least 20 minutes, at least 30 minutes, at least one hour, at least two hours, at least three hours greater than the half-life exhibited by otherwise identical EVs that lack at least one albumin.

The affinity purification method of the present invention comprises the steps of: (i) contacting a medium comprising the albumin-display EVs with a chromatography matrix comprising a corresponding receptor, for example FcRN, (ii) allowing the albumin-display EVs of the invention to adsorb to the FcRN, and (iii) eluting the albumin-display EVs by passing across the chromatography matrix a medium that releases the albumin-display EVs from the FcRN. As hereinbefore described, the EVs of the present invention are engineered to comprise and display on their surface albumin(s) such as, but not limited to the albumins given in SEQ ID NOs:1-2.

The principle of this process is what is known as affinity purification and/or affinity chromatography, i.e., purification of a particular target solute (in this case EVs, such as exosomes) from a complex biological fluid containing various types of solutes, based on the specific interaction between a generic ligand and a generic corresponding receptor, in this case an albumin (ligand) and a corresponding receptor, for example FcRN (receptor). The FcRN thereof is thus attached to a stationary phase, whereas the albumin protein is present on the EVs, which are comprised in the liquid phase, e.g., cell culture medium. The processes and methods of the present invention are easily applied to any type of cell culture medium, and various cell culture medium used for both adherent and suspension cells have been tested in the affinity chromatography methods of the present invention, for instance, RPMI, EMEM, DMEM, MEM, PMEM, PEM, Opti-MEM, IMDM, Advanced DMEM, McCoy's medium, medium with or without additives such serum, antibiotics, nutrients, etc.

In a further embodiment, the process may comprise triggering release of the EVs from the FcRN thereof by exposing the albumin-FcRN bond to a medium with a suitable pH. This is achieved by running the EV-containing medium (i.e., the liquid phase) through e.g., a chromatography column comprising, as stationary phase, a chromatography matrix having attached to it FcRN, letting the albumin protein of the EVs adsorb to the FcRN present on the matrix, and then running a solution with a suitable pH through the chromatography column. The pH of the solution that is intended to trigger release of the EVs from the column may be below pH 8, below pH 7, or below pH 6. Both the process of capturing the EVs and the process of releasing the EVs may be repeated multiple times, e.g., anywhere from once to up to e.g., 500 times.

In further embodiments, the FcRN or other suitable receptor may be attached to the chromatography matrix via different types of chemical and biochemical linkages and bonds. Covalent bonds between the matrix and the protein comprising the albumin protein, e.g., HSA, may be conventional amide bonds, disulfide bonds, ether bonds, esther bonds, thio-ether bonds, thio-esther bonds, glutathione-GST interactions, streptavidin-biotin interaction, etc. The matrix may be chemically activated to facilitate binding to the albumin protein or fragment/domain thereof using chemical conjugation moieties such as N-hydroxysuccinimide (NHS; for NHC-EDC/EDAC coupling), thiols, cyanogen bromide (CNBr), epoxy, thiopropyl, primary amines, sulfhydryls, carboxylic acids, aldehydes, iodoacetyl, azlactones, carbonyldiimidazole (CDI), maleimide, etc., as is well known to a person skilled in the art.

In preferred embodiments, the processes of the present invention are carried out in chromatography columns, which comprise the chromatography matrix comprising the albumin or fragment/domain thereof.

The chromatography matrix for use in capturing albumin-display EVs may consist of essentially any type of material suitable as a stationary chromatography phase. Non-limiting examples include one or more of agarose, dextran, lectin, heparin, cellulose, starch, dextran, agar, agarose, poly(meth)acrylate, polyacrylamide, polysulfone, a polyvinyl polymer, polystyrene, silica, alumina, zirconium oxide, titanium oxide, polysaccharide-mineral structure, polysaccharide-synthetic polymer, synthetic polymer-mineral structure, or any combination thereof. The matrix may be in the form of beads, fibers, irregularly shaped particles, membranes, flat structure, porous mineral materials or essentially any type of suitable stationary phase. Naturally, the albumin or fragment/domain thereof attached to the matrix may also be directly attached to various surfaces using chemical bonds and linkers. This could be particularly useful for methods such as e.g., surface plasmon resonance.

The affinity purification methods as per the present invention may further comprise additional EV purification step(s), which may be carried out prior to the affinity capture step(s) of the present invention. Suitable purification methods are as disclosed herein in connection with the second aspect of the invention.

Importantly, as above-mentioned, the affinity purification of albumin-display EVs may be run multiple times, essentially indefinitely, but at least anywhere between 2 and 500 times. Sequential purification of albumin-binding EVs enables exogenous drug loading between purification steps. For instance, albumin-binding EVs may be purified directly from the conditioned medium (CM) of the EV-producing cell source, followed by an exogenous drug loading step, and yet another round of purification. Schematically, this can be illustrated as follows:
Secretion of EVs into cell culture medium by EV-producing cells;
Affinity purification of EVs using the processes and methods of the present invention;
Drug loading (for instance, loading of siRNA into EVs/onto the surface of EVs by electroporation or transfection reagent);
Affinity purification of EVs using the processes and methods of the present invention.

The additional exogenous loading step may involve cargo being loaded using any known exogenous loading method including, but not limited to, the following: electroporation, transfection with transfection reagents such a cationic transfection agent, Lipofectamine®, conjugation of the cargo to a membrane-anchoring moiety, such as a lipid or cholesterol tail or tag, or loading by means of a CPP, either in the form of a CPP-cargo conjugate or in the form of a CPP-cargo non-covalent complex.

Any of the above aspects can be combined with any other aspect of the present invention.

Other features and advantages of the disclosure will be apparent from the following detailed description and claims.

The invention will be more clearly understood from the following description of some embodiments thereof, given by way of example only, and with reference to the Figures where appropriate.

All publications, patent applications, patents, and other references mentioned herein are incorporated by reference in their entirety. The references cited herein are not admitted as prior art to the claimed invention. In the case of conflict, the present specification, including definitions, will control.

### EXAMPLES

The materials, methods, and examples described hereinbelow detail embodiments according to the present invention and support the understanding thereof. In addition, the materials, methods, and examples are illustrative only and are not intended to be limiting in any way.

Although alternative methods and materials similar or equivalent to those described herein can be used in the practice or testing of the present disclosure, suitable methods and materials are described below.

### Example 1: Process of Engineering Albumin into an EV

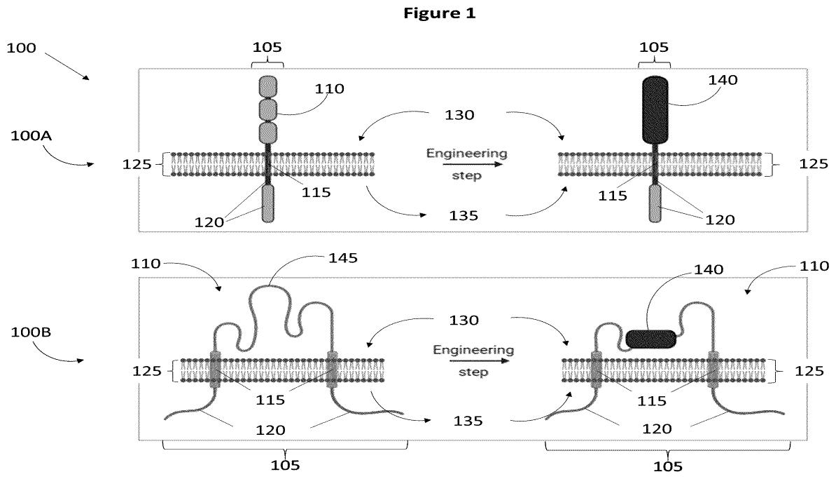

Referring to FIG. 1 there is there is provided an overview of engineering process for incorporating albumin into an EV to produce an albumin-display EV according to the embodiments of the present invention, indicated generally by reference numeral 100.

In one embodiment, there is provided terminal display of the albumin on the EV surface is shown in FIG. 1, indicated generally by 100A.

In this embodiment, there is shown a transmembrane protein 105 comprising an external domain 110, a transmembrane domain 115 and an internal domain 120. The transmembrane protein 105 spans the bilipid membrane of an EV 125, so that the external domain 110 of said protein 105 is on the outside of the EV 130 and the internal domain 120 of said protein 105 is on the inside of the EV 135.

Some examples of suitable proteins that could be utilised as a transmembrane protein 105 in the terminal display of albumin as herein described include but are not limited to Lamp2, TNFR and TfR1.

In this embodiment the EV undergoes an engineering step such that the external domain 110 of the transmembrane protein 105 is replaced with albumin 140. Display of albumin 140 on the surface of the EV (i.e., on the outside of the EV 130) is thus enabled.

In an alternative embodiment, an intraloop display of said albumin 130 in the external domain 110 of the transmembrane protein 105 on the EV surface is shown in FIG. 1, indicated generally by 100B. Parts similar to features hereinbefore described are accorded the same reference numeral.

In this embodiment, there is shown a transmembrane protein 105 comprising an external domain 110, a transmembrane domain 115 and an internal domain 120. The transmembrane protein 105 of this embodiment is a multi-pass transmembrane protein 105. The transmembrane protein 105 spans the bilipid membrane of an EV 125, so that the external domain 110 of said protein is on the outside of the EV and the internal domain 120 of said protein 105 is on the inside of the EV 135.

The external domain 110 of said protein 105 is shown to comprise an extravesicular loop 135 positioned on the outside of the EV between two transmembrane domains 115 spanning the bilipid membrane of the EV 125.

Some examples of suitable proteins that could be utilised as a multi-pass transmembrane protein in intraloop display of albumin as herein described include but are not limited to Limp2 and CD63.

In this embodiment the EV undergoes an engineering step such that a portion of the external domain 110 of the transmembrane protein 105 is replaced with albumin 140, wherein the albumin 140 is positioned about the extravesicular loop 145 of the external domain 110 of the transmembrane protein 105. Display of albumin 140 on the surface of the EV (i.e., on the outside of the EV 130) is thus enabled.

It will be appreciated that in alternative embodiments where a multi-pass transmembrane protein is utilised the transmembrane protein may pass the bilipid membrane of the EV at least 2, 3, 4, 5, 6, 7 or more times. With each of the passes 2, 4, 6 etc., of the transmembrane protein a further extravesicular loop is provided.

The albumin may be independently fused into each of a first, second, third, fourth or any subsequent loop of the multi-pass transmembrane protein or into more than one of said extravesicular loops. Alternatively, more than one albumin may be incorporated into one extravesicular loops.

It will be appreciated that while albumin is described as incorporated in the above example in other embodiments other albumin partial sequences, fragments and/or domains thereof may equally be employed provided they are capable of binding to any transmembrane protein of choice utilized in the EV of the present invention.

### Example 2: Albumin-Based Constructs for Incorporation into an EV

Nucleic acid fusion constructs encoding the desired fusion proteins were designed. According to the present invention, the nucleic acid fusion construct, and the corresponding protein sequence encoded for by said afore-mentioned nucleic acid fusion construct, comprises at least a signal peptide (SP), an albumin protein and a transmembrane polypeptide (TMP) in whole or in-part. In another embodiment, the nucleic acid fusion construct, and the corresponding protein sequence encoded for by said afore-mentioned nucleic acid fusion construct, comprises an exosome colocalization signal (ECS). In a further embodiment, the nucleic acid fusion construct, and the corresponding protein sequence encoded for by said afore-mentioned nucleic acid fusion construct, comprises a reporter. In a further embodiment, the nucleic acid fusion construct, and the corresponding protein sequence encoded for by said afore-mentioned nucleic acid fusion construct, comprises a propeptide (PP). It will be appreciated that a propeptide has utility in relocation and secretion of the final polypeptide produced. While polypeptides according to the present invention may comprise a propeptide sequence, it will be appreciated that this is strictly not a necessary component since EVs are employed. As such, a polypeptide absent the propeptide portion of said sequence would also equally work as part of the EVs as herein described. Optionally, additional motif/domain/protein sequences may also be included.

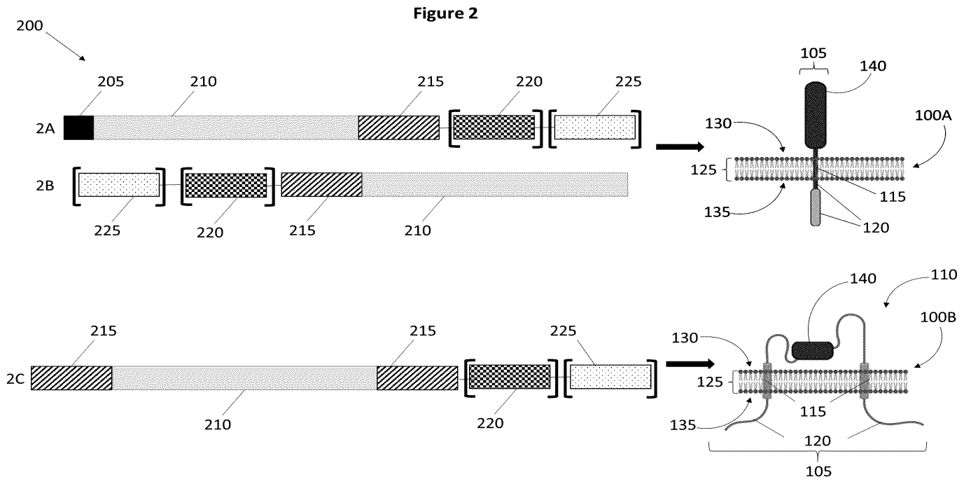

Referring to FIG. 2 there is there is provided an overview of exemplary nucleic acid fusion constructs and component parts according to the embodiments of the present invention, indicated generally by reference numeral 200. Parts similar to features hereinbefore described are accorded the same reference numeral.

Two alternative nucleic acid fusion constructs suitable for terminal display (FIGS. 2, 2A and 2B) of an albumin as described herein.

Referring now to FIG. 2, 2A there is provided display of the albumin on the N-terminal domain (NTD). A signal peptide (SP) sequence 205 is adjacent an albumin (Alb) sequence 210, the Alb sequence 210 is adjacent a transmembrane polypeptide (TMP) sequence 215. The TMP 215 is adjacent an exosome co-localization signal (ECS) sequence 220. The ECS 220 is adjacent a reporter 225. Both the ECS 220 and the reporter 225 are shown in parenthesis, meaning that these features are considered optional in the overall design of the nucleic acid fusion construct as hereinbefore described. In this embodiment, as shown, the core structure of the nucleic acid fusion construct thus comprises SP 205, Alb 210 and TMP 215. Optionally, ECS 220 and reporter 225 may be added as required.

While not shown, it will be appreciated that additional motif/domain/protein sequences may be included.

In use, the hereinbefore described nucleic acid fusion construct localises at the endoplasmic reticulum and the SP initiates synthesis of the corresponding polypeptide sequence as encoded for by the hereinbefore described nucleic acid fusion construct. The signal peptide and/or the propeptide, if present, may be cleaved off as part of post translational modification to arrive at the finished polypeptide sequence.

The resulting corresponding polypeptide sequence from the hereinbefore described nucleic acid fusion construct, will share a corresponding structure to the structure as defined by said nucleic acid fusion construct.

Some exemplary examples of constructs include, but are not limited to the following:
SP-HSA-LAMP2B
SP-PP-HSA-LAMP2B
SP-PP-HSA-LAMP2B-ECS
SP-PP-HSA-LAMP2B-ECS-NLuc
SP-DIII-LAMP2B
SP-PP-DIII-LAMP2B
SP-PP-DIII-LAMP2B-ECS
SP-PP-DIII-LAMP2B-ECS-NLuc
SP-HSA-TfR1
SP-PP-HSA-TfR1
SP-PP-HSA-TfR1-ECS
SP-PP-HSA-TfR1-ECS-NLuc
SP-DIII-TfR1
SP-PP-DIII-TfR1
SP-PP-DIII-TfR1-ECS
SP-PP-DIII-TfR1-ECS-NLuc

The NTD is not shown in the context of the vesicular membrane in FIG. 2, 2A but it will be appreciated that the NTD in this construct would be intended to present the albumin protein on the surface (outside) of the EV in the resulting polypeptide sequence derived from this construct.

Referring now to FIG. 2, 2B there is provided display of the albumin on the C-terminal domain (CTD). Parts similar to features hereinbefore described are accorded the same reference numeral.

A reporter 225 is shown as adjacent an ECS sequence 220, the ECS 220 is adjacent a TMP sequence 215. The TMP 215 is adjacent an albumin (Alb) sequence 210.

Both the ECS 220 and the reporter 225, in this embodiment of the present invention, as shown, are in parenthesis, meaning that these features are considered optional in the overall design of the nucleic acid fusion construct as hereinbefore described. In this embodiment, as shown, the core structure of the nucleic acid fusion construct thus comprises Alb 210 and TMP 215. Optionally, ECS 220 and reporter 225 may be added as required.

While not shown, it will be appreciated that additional motif/domain/protein sequences may be included.

In use, the hereinbefore described nucleic acid fusion construct localises at the endoplasmic reticulum and initiates synthesis of the corresponding polypeptide sequence as encoded for by the hereinbefore described nucleic acid fusion construct.

The resulting corresponding polypeptide sequence from the hereinbefore described nucleic acid fusion construct, will share a corresponding structure to the structure as defined by said nucleic acid fusion construct.

Some exemplary examples of constructs include, but are not limited to the following:
LAMP2B-HSA
ECS-LAMP2B-HSA
ECS-NLuc-LAMP2B-HAS
LAMP2B-DIII
ECS-LAMP2B-DIII
ECS-NLuc-LAMP2B-DIII
TfR1-HSA
ECS-TfR1-HSA
ECS-NLuc-TfR1-HSA
TfR1-DIII
ECS-TfR1-DIII
ECS-NLuc-TfR1-DIII

The CTD is not shown in the context of the vesicular membrane in FIG. 2, 2B but it will be appreciated that the CTD in this construct would be intended to present the albumin protein on the surface (outside) of the EV in the resulting polypeptide sequence derived from this construct.

The inventors envisage certain embodiments of the present invention, wherein both albumin termini are associated with an extravesicular loop of a TM polypeptide that is displayed on the outer surface of an EV in accordance with the present invention.

Referring now to FIG. 2, 2C there is provided display of an albumin protein by means of intraloop display. Parts similar to features hereinbefore described are accorded the same reference numeral.

As shown and reading from left to right of the schematic, a TMP sequence 215 is adjacent an albumin (Alb) sequence 210, the Alb sequence 210 is adjacent another TMP sequence 215. Such that Alb 210 is sandwiched between the two TMP sequences 215.

Only two TMP 215 are shown in FIG. 2, 2C, one at each termini of the Alb 210, to sandwich the Alb sequence 210 as hereinbefore described. However, the inventors envisage that two or more TMP 215 at each termini position of the Alb sequence 210 may be incorporated into the nucleic acid fusion construct during the design phase.

One advantage of using TMP's in whole or in-part is that variation in the way albumin is displayed on the surface of the EV is enabled. As such the exact display topography is characterised by the length and portion of the TMP the placement of said length and portion of the TMP relative to the albumin in the fusion construct.

Another advantage of using TMP's in whole or in-part is that use of multi-pass TM proteins such as Lysosomal membrane protein type-2 (Limp2; 2 TMD) and tetraspanins (four TMD) are enabled in that the length and portion of the TMP may relate to

A further advantage of two or more TMP's is that stability of the transmembrane protein may be maintained by retaining the original TMPs of that protein.

Both the ECS 220 and the reporter 225 are shown in parenthesis, meaning that these features are considered optional in the overall design of the nucleic acid fusion construct as hereinbefore described. In this embodiment, as shown, the core structure of the nucleic acid fusion construct thus comprises TMP 215, Alb 210 and TMP 215. Optionally, ECS 220 and reporter 225 may be added as required.

These optional components are shown in FIG. 2, 2C as positioned to the right of the other components of the nucleic acid fusion construct and adjacent the second TMP 215. However, it will be appreciated that the nucleic acid fusion construct may either start and/or end with a reporter 225.

While not shown, it will be appreciated that additional motif/domain/protein sequences may be included.

In use, the hereinbefore described nucleic acid fusion construct localises at the endoplasmic reticulum and initiates synthesis of the corresponding polypeptide sequence as encoded for by the hereinbefore described nucleic acid fusion construct.

The resulting corresponding polypeptide sequence from the hereinbefore described nucleic acid fusion construct, will share a corresponding structure to the structure as defined by said nucleic acid fusion construct.

It will be appreciated that, where the hereinbefore described nucleic acid fusion construct either starts, ends, or both starts and ends with a reporter 225, the entire sequence resulting from said nucleic acid fusion construct may be either initiated or terminated with a polypeptide sequence that corresponds to the reporter 225 of the hereinbefore described nucleic acid fusion construct.

The hereinbefore described nucleic acid fusion constructs are exemplary and should not be considered to limit the invention in any way. In particular, and while it is not shown, it will be appreciated that other alternative arrangements of the component parts of said nucleic acid fusion constructs, other than that hereinbefore described, are readily envisaged by the inventors. Such alternative arrangements may be routinely designed and may be suitably employed, without departing from the present invention as hereinbefore described.

Some exemplary examples of constructs include, but are not limited to the following:
LIMP2-HSA-LIMP2
LIMP2-HSA-LIMP2-ECS
LIMP2-HSA-LIMP2-ECS-NLuc
LIMP2-DIII-LIMP2
LIMP2-DIII-LIMP2-ECS
LIMP2-DIII-LIMP2-ECS-NLuc

### Example 3: Expression of Fusion Constructs in CAP Cells

Nucleic acid fusion constructs encoding the desired fusion proteins were designed. Said nucleic acid fusion constructs are described in the Key below.

### Key:

hCD63-Nluc (SEQ ID NO: 14): encodes for a control fusion protein formed of an exosome protein (hCD63) and expressing nanoluc reporter (lacking albumin protein).
hAlb-LAMP2B-Nluc (fusion comprising SEQ ID No: 8, SEQ ID No: 10 and 1 SEQ ID No: 5): encodes a fusion protein formed of exosomal protein (LAMP2B) with human albumin engineered to be terminally displayed on the surface of the EVs and expressing nanoluc reporter.
Trunc.LIMP2-hAlb-Nluc (fusion comprising a truncated version of SEQ ID No: 11, SEQ ID No: 8 and SEQ ID No: 15): encodes for a fusion protein formed of exosomal protein (Trunc.LIMP2) with human albumin engineered for intraloop display on the surface of the EVs and expressing nanoluc reporter.

The above nucleic acid fusion constructs were then introduced into EV producing cells (i.e., producer cell/cell source), such as CAP cells, using technology as disclosed herein, so that those cells expressed the fusion protein. This fusion protein was then incorporated into the EVs produced by the CAP cell, due to the presence of the EV protein in the fusion construct that is introduced to the CAP cells. Expression of said constructs in stably transduced CAP cells was tested using western blot.

### Cell Culture of CAP Cells

In this example CAP cells were used as the producer cell/cell source for the EVs according to the present invention. CAP cells were cultured employing conventional methods. For example, the CAP cells were suspended in media at 37° C., 5% CO₂ with shaking in a humidified incubator.

While not described, it will be appreciated that conventional methods used are well known and other producer cells/cell sources may equally be employed. Inventors envisage that said methods can be routinely adapted to any suitable producer cell type used by routine modification of, for example, the media composition and the conditions under which the cells are grown.

### Transfection of CAP Cells

One day prior to the transfection, CAP cells were seeded to 1E6/ml.

For EV production, 1.6E9 of the seeded CAP cells were initially washed in PBS and then resuspended in 120 ml transfection medium.

CAP cells were then transfected with 600 ug DNA lipolexed in 1200 ug PEI and topped up to 600 ml with any one of several suitable conventional media available.

Exosome Preparation from CAP Cells

CAP cells were then subjected to differential centrifugation to clear cellular debris from the conditioned medium. The conditioned medium was concentrated by tangential flow filtration (TFF) and cleared using a multimodal resin. Material underwent a final concentration step by TFF.

### Western Blot Methodology

Conventional western blots (WB) were performed to assess various fusion constructs comprising albumin or its DIII domain (data not shown). A second WB confirmed the choice of preferred constructs. Details of said WB, specific to the present invention are hereinafter described.

Samples comprising 1E10 EVs or 30 ug whole cell lysate (WCL) were prepared for SDS-PAGE. Following SDS-PAGE proteins were transferred onto a PVDF membrane and blocked and probed with primary antibodies for the markers Nanoluc (R&D systems MAB100261) and syntenin (Abcam Ab133267). Secondary antibodies to the primary antibodies were obtained from Licor (216778 and 216775). WCLs were subject to a Western Blot using anti-NanoLuc antibody (Anti-NLuc Ab) to determine the expression of NanoLuc, which indicates the loading levels of albumin in CAP cells. Syntenin was used as internal control.

While not described in the present method, it will be appreciated that other suitable markers may equally be probed for and employed.

EV species are defined by their nucleic acid fusion constructs as follows: hCD63-Nluc (SEQ ID NO: 14), hAlb-LAMP2B-Nluc (SEQ ID No: 8 and SEQ ID No: 10), Trunc.LIMP2-hAlb-Nluc (SEQ ID No: 11, SEQ ID No: 8 and SEQ ID No: 15). Detailed descriptions hereinabove described.

The corresponding fusion proteins are as follows: hCD63-Nluc (SEQ ID NO: 7), hAlb-LAMP2B-Nluc (fusion protein comprising SEQ ID No: 1 and SEQ ID No: 3), Trunc.LIMP2-hAlb-Nluc (fusion protein comprising truncated version of SEQ ID No: 4 and SEQ ID No: 1).

The constructs and resulting fusion proteins of Example 3 include nanoluc. The bioluminenscent label, Nanoluc, is included in this embodiment, to measure the various fusion proteins in the WB. Fusion proteins are also envisaged where no nanoluc is included.

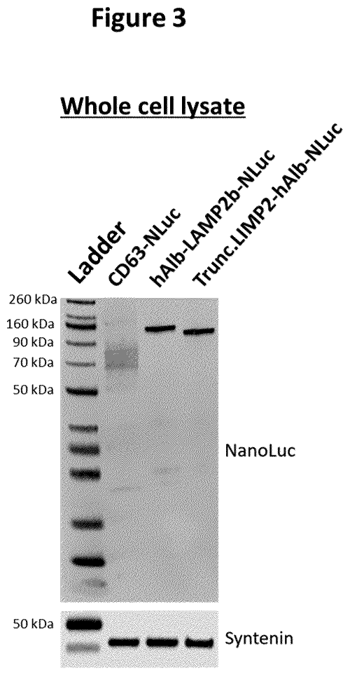

Referring to FIG. 3 human albumin protein expression is shown across different EV species according to the present invention compared to control fusion protein (CD63-Nluc; SEQ ID No: 7).

As shown in FIG. 3, syntentin fluorescence observed confirmed a good protein transfer to the WB membrane. Nanoluc fluorescence observed confirmed that constructs comprising human albumin fused to the terminus of a single-pass transmembrane protein (LAMP2B; SEQ ID NO: 3) was incorporated well into the CAP cells. Nanoluc fluorescence observed also confirmed that constructs comprising albumin fused to an intraloop portion of an extravesicular loop of a multi-pass transmembrane protein (trunc.LIMP2; SEQ ID NO: 4) was incorporated well into the CAP cells.

### Example 4: Expression of Fusion Constructs in EV Fraction

Once the expression of the nucleic acid fusion constructs in stably transduced CAP cells was established, the expression of the nucleic acid fusion constructs in the EV fraction after purification was then tested by western blot. Fusion constructs are described in the Key below.

### Key:

hCD63-Nluc (SEQ ID NO: 14): encoding for a control fusion protein of an EV protein (hCD63) and nanoluc reporter (i.e., lacking albumin).
hAlb-TNFR-Nluc (fusion construct comprising SEQ ID No: 8, SEQ ID No: 12 and SEQ ID No: 15): encoding for a fusion protein of an EV protein (TNFR) with human albumin engineered to be terminally displayed on the surface of the EVs and expressing nanoluc reporter.
DIII-LAMP2B-Nluc (fusion construct comprising SEQ ID No: 9, SEQ ID No: 10 and SEQ ID No: 15) encoding for a fusion protein of EV protein (LAMP2B) with albumin domain DIII engineered to be terminally displayed on the surface of the EVs and expressing nanoluc reporter.
hAlb-LAMP2B-Nluc (fusion construct comprising SEQ ID No: 8, SEQ ID No: 10 and SEQ ID No: 15): encoding for a fusion protein of EV protein (LAMP2B) with human albumin engineered to be terminally displayed on the surface of the EVs and expressing nanoluc reporter.
LIMP2-DIII-Nluc (fusion construct comprising SEQ ID No: 11, SEQ ID No: 9 and SEQ ID No: 15): encoding for a fusion protein of EV protein (LIMP2) with albumin domain DIII engineered for intraloop display on the surface of the EVs and expressing nanoluc reporter.
Trunc.LIMP2-DIII-Nluc (fusion construct comprising truncated version of SEQ ID No: 11, SEQ ID No: 9 and SEQ ID No: 15): fusion protein of EV protein (Trunc.LIMP2) with albumin domain DIII engineered for intraloop display on the surface of the EVs and expressing nanoluc reporter.
Trunc.LIMP2-hAlb-Nluc (fusion construct comprising a truncated version of SEQ ID No: 11, SEQ ID No: 8 and SEQ ID No: 15): fusion protein of EV protein (Trunc.LIMP2) with human albumin engineered for intraloop display on the surface of the EVs and expressing nanoluc reporter.

Samples were prepared and WB performed according to the methods of Example 3 adapted as follows:

Nanoluc, TSG101, CD81 and GN130 primary antibodies were used respectively for detection. Both TSG101 and CD81 are exosomal marker proteins, Nanoluc is a bioluminescent tag and GN130 is a golgi marker as a negative control. The albumin used in the protein fusion constructs of Example 4 were hAlb (SEQ ID NO: 1) and DIII (SEQ ID NO: 2).

EV species are defined by their nucleic acid fusion constructs as follows: hCD63-Nluc (SEQ ID NO: 14), hAlb-TNFR-Nluc (SEQ ID NOs: 8, 12, 15), DIII-LAMP2B-Nluc (SEQ ID NOs: 9, 10, 15) hAlb-LAMP2B-Nluc (SEQ ID Nos: 8, 10, 15), LIMP2-DIII-Nluc (SEQ ID NOs: 11, 9, 15), Trunc.LIMP2-DIII-Nluc (SEQ ID NOs: 11, 9, 15), Trunc.LIMP2-hAlb-Nluc (SEQ ID Nos: 11, 8, 15). Detailed descriptions hereinbefore described.

The corresponding fusion proteins are as follows: hCD63-Nluc (SEQ ID NO: 7), hAlb-TNFR-Nluc (SEQ ID NOs: 1 and 5), DIII-LAMP2B-Nluc (SEQ ID NOs: 2 and 3) hAlb-LAMP2B-Nluc (SEQ ID Nos: 1 and 3), LIMP2-DIII-Nluc (SEQ ID NOs: 4 and 2), Trunc.LIMP2-DIII-Nluc (truncated version of SEQ ID NO: 4 and SEQ ID NO: 2), Trunc.LIMP2-hAlb-Nluc (truncated version of SEQ ID No: 4, and SEQ ID No: 2).

The constructs and resulting fusion proteins of Example 4 include nanoluc. The bioluminenscent label, Nanoluc, is included in this embodiment, to trace and measure the various fusion proteins in the WB. Fusion proteins are also envisaged where no nanoluc is included.

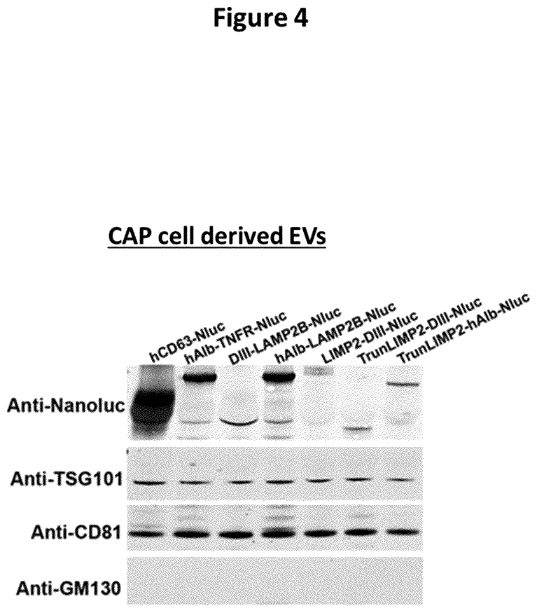

Referring to FIG. 4, all the constructs were shown to be expressed well in EVs purified from stable CAP cells. The expression of the constructs did not decrease after several passages.

A further WB was performed wherein EVs were subject to anti-NanoLuc antibody to determine the protein expression of NanoLuc which indicates the loading levels of Albumin in each engineered species. Syntenin was used as a loading control.

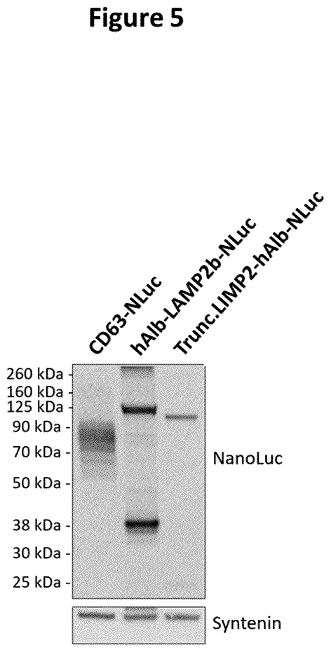

Referring now to FIG. 5, which shows the output from the secondary WB. Constructs were confirmed to be expressed well in EVs purified from stable CAP cells.

### Example 5: Quality of Engineered Albumin-Display EVs

Albumin-display EVs in accordance with the present invention, were prepared from CAP cells according to the methods of Example 3. The CAP-cell derived albumin-display EVs were then lysed and the contents subject to conventional size exclusion.

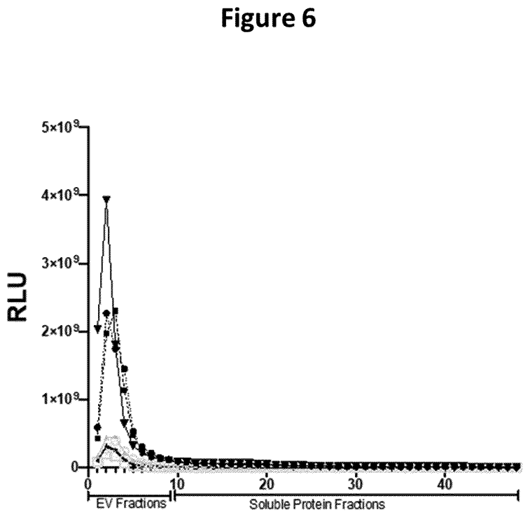

Referring to FIG. 6 a plot of Relative Light Units (RLUs) for EV fractions and other soluble protein fractions within the EV lysate. The lysate of several albumin-display EVs including (hAlb-TNFR-Nanoluc ( [symbol] ), DIII-LAMP2B-Nanoluc ( [symbol] ), hAlb-LAMP2B-Nanoluc ( [symbol] ), LIMP2-DIII-Nanoluc ( [symbol] ), trunc.LIMP2-DIII-Nanoluc ( [symbol] ) and trunc.LIMP2-hALb-Nanoluc ( [symbol] )) and control EVs (hCD63-Nanoluc ( [symbol] )) is shown.

The CAP-derived albumin-display EVs were well-engineered, as shown by FIG. 6, where the nanoluc signals (expressed as RLU) were only detected in the EV fractions after size exclusion.

### Example 6: Improved Half-Life of Albumin-Display EVs In-Vivo

Albumin-display EVs in accordance with the present invention, were prepared from CAP cells according to the methods of Example 3. The half-life of the albumin-display EVs was then tested in vivo in NMRI mice compared to EVs lacking albumin display.

EVs that were engineered to express human albumin or albumin DIII on the surface plus the luminescent nanoluc reporter (hAlb-TNFR-Nanoluc (SEQ ID NOs: 1 and 5), DIII-LAMP2B-Nanoluc (SEQ ID NOs: 2 and 3), hAlb-LAMP2B-Nanoluc (SEQ ID NOs: 1 and 3), LIMP2-DIII-Nanoluc (SEQ ID NOs: 4 and 2), trunc.LIMP2-DIII-Nanoluc (truncated SEQ ID NO: 4 and SEQ ID NO: 2) and trunc.LIMP2-hALb-Nanoluc (truncated SEQ ID NO:4 and SEQ ID NO: 1)) and control EVs engineered to only express nanoluc (hCD63-Nanoluc (SEQ ID NO:7)) were compared. The albumin or albumin DIII used in the constructs of Example 4 were hAlb (SEQ ID NOs: 1, 8) and DIII (SEQ ID NOs: 2, 9).

The constructs and resulting fusion proteins of Example 6 include nanoluc. The bioluminenscent label, Nanoluc, is included in this embodiment, to trace and measure the various fusion proteins in vivo. Fusion proteins are also envisaged where no nanoluc is included.

Five NMRI mice per group were used and EVs were injected intravenously at a dose of 1E11/mouse. Bioluminescence (Nluc) in the plasma was measured with raw data expressed as. Relative Light Units (RLU). The number of EVs remaining in circulation at a certain time point is calculated based on Relative Light Unit (RLU)/injected EV number (Total RLU/1E11). From this, the percentage of injected EVs in plasma is derived.

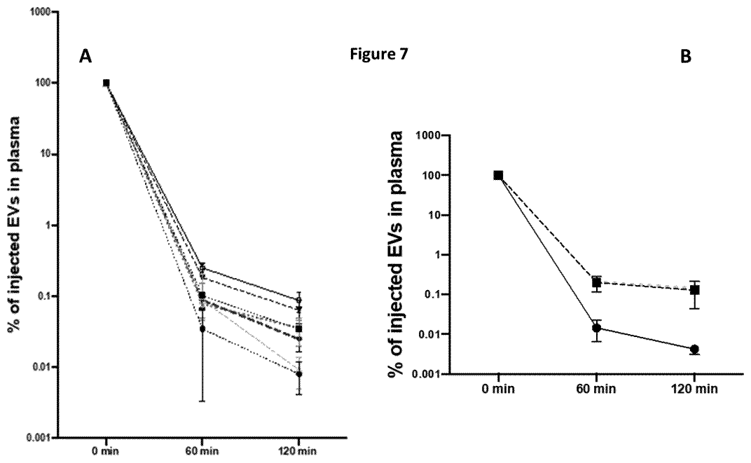

Referring to FIG. 7A, the percentage of injected EVs in plasma is plotted against time (0 m, 60 m, 120 m post injection). The half-life of several albumin-display EVs (hAlb-TNFR-Nanoluc ( [symbol] ), DIII-LAMP2B-Nanoluc ( [symbol] ), hAlb-LAMP2B-Nanoluc ( [symbol] ), LIMP2-DIII-Nanoluc ( [symbol] ), trunc.LIMP2-DIII-Nanoluc ( [symbol] ) and trunc.LIMP2-hALb-Nanoluc ( [symbol] )) and control EVs (hCD63-Nanoluc ( [symbol] )) is shown. As is shown by FIG. 6A all albumin-display EVs tested exhibited an improved half-life over control. Albumin-display EVs hAlb-LAMP2B-Nanoluc ( [symbol] ) and trunc.LIMP2-hALb-Nanoluc ( [symbol] ) had particularly good half-life with more than 10 times more circulating EVs over control.

Post injection half-life experiments were repeated for selected albumin display EVs hAlb-LAMP2B-Nanoluc (SEQ ID NOs: 1 and 3) and trunc.LIMP2-hALb-Nanoluc (truncated SEQ ID NO:4 and SEQ ID NO: 1)) and control EVs (hCD63-Nanoluc (SEQ ID NO:8)) according to same methodology as hereinbefore described.

Referring now to FIG. 7B, the percentage of injected EVs in plasma is plotted against time (Cm, 60 m, 120 m post injection). The half-life of the preferred albumin-display EVs hAlb-LAMP2B-Nanoluc ( [symbol] ) and trunc.LIMP2-hALb-Nanoluc ( [symbol] )) and control EVs (hCD63-Nanoluc ( [symbol] )) is shown. Improved half-life of the selected albumin-display EVs over control was confirmed. The inventors even observed that the half-life extension effect of these albumin-display EVs was more significant than the first half-life experiment conducted, as hereinbefore described, meaning that, on repeat the data even more convincing shows the half-life to be improved.

Referring more generally to FIG. 7, the display of albumin on the surface of an EV is shown to significantly increase the half-life of EVs in circulation, by making the EVs more stable at least up to the 2 h time point.

The significant drop in circulating EVs within the first hour post injection is thought to be due to uptake by phagocytic cells (monocytes and macrophages) in circulation. The tail off in circulating EVs observed during the second hour post injection is thought be due to saturation of said phagocytic cells, which after the first hour may be working less efficiently to clear any remaining EVs from circulation. The difference in gradient of the line plot between albumin-display EVs and control indicates that albumin-display EVs are not as readily removed from circulation by said phagocytes.

### Example 7: Biodistribution of Albumin-Display EVs Against Control

EVs are known to be predominantly taken up by the liver and the spleen, meaning that targeting organs other than the hepatosplenic system is limited without further modification. This is one problem that the present invention seeks to overcome. As such, it is desirable to divert EVs away from uptake by the liver and the spleen so that delivery to other organs is increased, thus improving biodistribution.

The inventors hypothesized that the improved half-life of EVs according to the present invention would impact on the biodistribution of said EVs. To investigate the biodistribution of albumin-display EVs the accumulation of exemplary albumin-display proteins according to the present invention (protein fusion comprising hAlb-TNFR-NanoLuc (SEQ ID NOs: 1 and 5), DIII-LAMP2B-NanoLuc (SEQ ID NOs: 2 and 4), hAlb-LAMP2B-NanoLuc (SEQ ID NOs: 1 and 3), LIMP2-DIII-NanoLuc (SEQ ID NOs: 4 and 2), Trunc.LIMP2-DIII-NanoLuc (truncated SEQ ID NO: 4 and SEQ ID NO: 2), trunc.LIMP2-hAlb-NLuc (truncated SEQ ID NO: 4 and SEQ ID NO:1)) were compared to control EVs (protein fusion hCD63-NanoLuc (SEQ ID NO: 8)) across a selection of organs.

The constructs and resulting fusion proteins of Example 7 include nanoluc. The bioluminenscent label, Nanoluc, is required to trace and measure the various fusion proteins in vivo. Fusion proteins are also envisaged where no nanoluc is included.

Albumin-display EVs in accordance with the present invention, were prepared from CAP cells according to the methods of Example 3. Each CAP-derived EV species was prepared for IV administration and was injected, via the tail vein, of NMRI mice.

Blood was then sampled, and the internal organs were harvested at 270 mins post injection. The albumin used in the constructs of Example 7 were hAlb (SEQ ID NOs: 1, 8) and DIII (SEQ ID NOs: 2, 9). Bioluminescence (Nluc) was measured and expressed as RLUs. Total RLUs in each organ was measured and the percentage of injected EVs was calculated based on RLU/injected EV number (Total RLU/1E11).

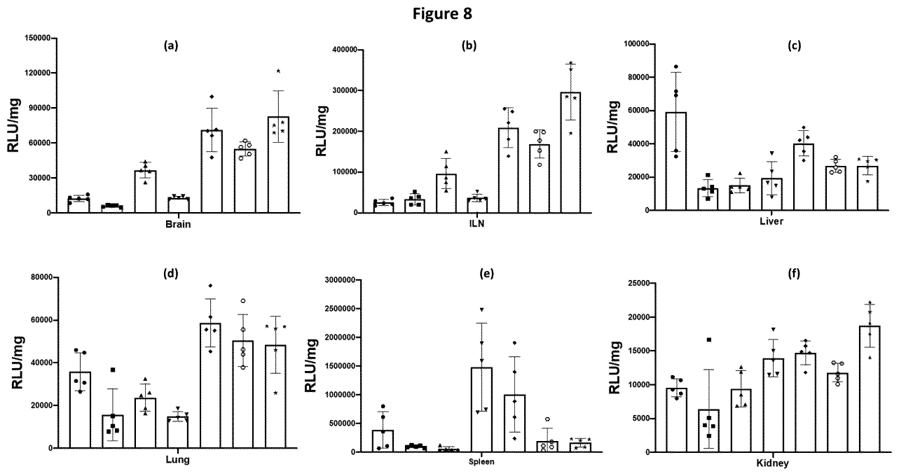

Referring generally to FIG. 8, the biodistribution profile of albumin-display EVs across a selection of organs is show.

Referring to FIG. 8(a), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) in the brain is shown.

The inventors found that Nluc bioluminescence was increased as compared to control samples, in the brain from mice that were administered the albumin-display EVs. The bioluminescence was particularly elevated in the albumin-display EV species LIMP2-DIII-Nanoluc (♦), trunc.LIMP2-DIII-Nanoluc ( [symbol] ) and trunc.LIMP2-hALb-Nanoluc (★).

The inventors concluded that accumulation of albumin-display EVs were significantly increased in the brain. This is especially so when a multi-pass transmembrane EV protein forms part of the fusion protein with the albumin protein.

Uptake into the brain is challenging and many therapeutics are hampered by their inability to cross the blood brain barrier (BBB) limiting their utility as a therapeutic. The inventors have surprisingly found that the EVs according to the present invention, especially where a multi-pass transmembrane protein is used, have the capability to cross the BBB, meaning that the EVs according to the present invention will greatly increase the utility of any therapeutic EVs which incorporate the inventive features of the present invention.

Referring now to FIG. 8(b), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) in the ILN is shown.

The inventors found that Nluc bioluminescence was increased in the Inguinal lymph nodes (ILN) from mice that were administered the albumin-display EVs as compared to control samples. The bioluminescence was particularly elevated in the albumin-display EV species LIMP2-DIII-Nanoluc (♦), trunc.LIMP2-DIII-Nanoluc ( [symbol] ) and trunc.LIMP2-hALb-Nanoluc (★). The inventors concluded that accumulation of albumin-display EVs were significantly increased in the ILN. This is especially so when a multi-pass transmembrane EV protein forms part of the fusion protein with the albumin protein.

Uptake into the lymph nodes is challenging and many therapeutics are hampered by their inability to accumulate at the lymph nodes, limiting their utility as a therapy and, specifically, as a cancer immunotherapy. The inventors have surprisingly found that the EVs according to the present invention, especially where a multi-pass transmembrane protein is used, have the capability to accumulate in the lymph nodes, meaning that the EVs according to the present invention will greatly increase the utility of any therapeutic EVs which incorporate the inventive features of the present invention, especially utility as a cancer immunotherapy.

Referring now to FIG. 8(c), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) in the Liver is shown.

The inventors found that Nluc bioluminescence was decreased in the liver from mice that were administered the albumin-display EVs versus control. The inventors concluded that accumulation of albumin-display EVs were significantly decreased in the liver. The data demonstrates that the EVs according to the present invention are diverted away from the liver, a major source by which EVs are excreted by the subject. Diverting EVs away from uptake by the liver enables increased delivery to other, harder to reach, organs, and thus improves biodistribution profile of the EVs.

The inventors have surprisingly found that the EVs according to the present invention, as a result of having an improved biodistribution profile, will greatly increase the utility of any therapeutic EVs which incorporate the inventive features of the present invention.

Referring to FIG. 8(d), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) the Lung is shown.

The inventors found that Nluc bioluminescence was increased as compared to control in the lung from mice that were administered the albumin-display EV species LIMP2-DIII-Nanoluc (♦), trunc.LIMP2-DIII-Nanoluc ( [symbol] ) and trunc.LIMP2-hALb-Nanoluc (★). Bioluminescence was deteriorated in the lung from mice that were administered the albumin-display EV species hAlb-TNFR-Nanoluc (▪), DIII-LAMP2B-Nanoluc (▴), hAlb-LAMP2B-Nanoluc (▾).

The inventors concluded that accumulation of several albumin-display EV species was decreased in the Lung while accumulation was increased in other albumin-display EV species. The data demonstrates that the EVs according to the present invention are diverted away from the lung, a major source by which EVs are excreted by the subject. Diverting EVs away from uptake by the lung enables increased delivery to other, harder to reach, organs, and thus improves biodistribution profile of the EVs.

The inventors have surprisingly found that the EVs according to the present invention, as a result of having an improved biodistribution profile, will greatly increase the utility of any therapeutic EVs which incorporate the inventive features of the present invention. This is especially so where a single-pass transmembrane EV protein forms part of the fusion protein with the albumin protein.

Referring to FIG. 8(e), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) the spleen is shown.

The inventors found that Nluc bioluminescence was increased as compared to control, in the spleen from mice that were administered the albumin-display EV species hAlb-LAMP2B-Nanoluc (▾), LIMP2-DIII-Nanoluc (♦). The inventors found that Nluc bioluminescence was about the same as compared to control, in the spleen from mice that were administered the albumin-display EV species trunc.LIMP2-DIII-Nanoluc ( [symbol] ) and trunc.LIMP2-hALb-Nanoluc (★). Bioluminescence was decreased in the spleen from mice that were administered the albumin-display EV species hAlb-TNFR-Nanoluc (▪), DIII-LAMP2B-Nanoluc (▴).

The inventors concluded that accumulation of several albumin-display EV species was decreased in the spleen while accumulation was increased in other albumin-display EV species. The data demonstrates that the EVs according to the present invention are moderately diverted away from the spleen, a major source by which EVs are excreted by the subject. Diverting EVs away from uptake by the spleen enables increased delivery to other, harder to reach, organs, and thus improves biodistribution profile of the EVs.

The inventors have surprisingly found that the EVs according to the present invention, as a result of having an improved biodistribution profile, will greatly increase the utility of any therapeutic EVs which incorporate the inventive features of the present invention. However, the choice of components making up the fusion protein would require careful consideration if diversion away from the spleen is required. The EVs according to the present invention remain a

Referring to FIG. 8(f), accumulation of several albumin-display EVs (hAlb-TNFR-Nanoluc (▪), DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★)) and control EVs (hCD63-Nanoluc ( [symbol] )) in the kidney is shown.

The inventors found that Nluc bioluminescence was marginally increased in the kidney from mice that were administered the albumin-display EVs. DIII-LAMP2B-Nanoluc (▴), hAlb-LAMP2B-Nanoluc (▾), LIMP2-DIII-Nanoluc (♦), trunc.LIMP2-DIII-Nanoluc ( [symbol] ) and trunc.LIMP2-hALb-Nanoluc (★). Bioluminescence was marginally decreased in the kidney from mice that were administered the albumin-display EV species hAlb-TNFR-Nanoluc (▪).

The inventors concluded that the EV accumulation in kidney did not significantly change with the type of albumin-display EV species administered.

Referring again more generally to FIG. 8, the presence of albumin protein is demonstrably beneficial for altering the biodistribution of EVs. This is especially true for EVs where the intended target organ is the brain or for the treatment of cancer, where high levels of EVs are desirable in the lymph nodes.

Moreover, while not shown, it will be appreciated that EVs in accordance with the present invention would be even more useful where EVs according to the present invention additionally comprise a targeting moiety; for instance, EVs which are engineered to comprise a targeting moiety that targets a particular organ or disease state of an organ, such as brain-targeted EVs or EVs comprising cancer targeting moieties.

### Example 8: Storage Stability of Albumin-Display EVs

Therapeutic EVs are commonly stored frozen at −20° C. or −80° C. The shelf-life of EVs stored at either of said temperatures is at most 1 year. This limits the accessibility of therapeutic EVs.

As shown from the examples above, the presence of albumin protein on the surface of EVs can increase the half-life, of EVs in vivo. This is attributable to the ability of the albumin protein according to the present invention to provide a corona about the EVS that act as a protective shield against environmental factors.

The inventors thus anticipate that the presence of the albumin protein according to the present invention may equally be advantageous to improve the stability of EVs in storage.

It will be appreciated that albumin-display EVs according to the present invention can be stored in any suitable storage buffer for said EVs.

To investigate this hypothesis, a cross-section of albumin-display EVs according to the present invention were added to buffer. Buffer lacking an albumin-display EV and/or buffer containing non-engineered EVs were used as control. Furthermore, to test if addition of albumin to the storage buffer imparts any additional benefit a cross-section of albumin-display EVs according to the present invention were added to an albumin enriched buffer.

Stocks of EVs are typically stored in a freezer, when not in use. As such preparations containing EVs are subject to freeze thaw. Display of albumin protein on the surface of the EVs according to the present invention coat the outer surface of the EV providing a protective shield of albumin protein. Such a protective shield is believed to protect the EVs against damage caused by freeze thaw cycles and thus improve the stability in formulation in addition to improving half-life in vivo. This stability is anticipated to advantageously result in a proven increase shelf-life of the pharmaceutical composition of the EVs, making a more robust versatile product that will remain bioactive for longer during storage.

Albumin-display EVs are obtained from an EV-producing genetically engineered and immortalized cell line cultured in bioreactors in conditioned medium (CM). CM containing the EVs is harvested from the bioreactors. EVs are then isolated from the CM by centrifugation, to remove cells and cell debris, and thereafter filtrated to remove any larger particles. The filtered CM is then run through a hollow fiber filter using a TFF system and concentrated down after diafiltration. The EVs are then combined with a formulation buffer comprising albumin and this formulation is then either a) stored for a predetermined period (at different temperatures (−80° C., −20° C., 4° C.) for up to 30 weeks); or b) subjected to repeated freeze thaw cycles.

The quality and robustness of the EV population is then tested following the prolonged periods of time in storage at different temperatures as well as the repeated freeze thaw cycles. EV number is tested using nanoparticle tracking analysis (NTA). The quality of the EVs is measured using several techniques:
i) by comparing the RNA content before and after storage/stress testing;
ii) EVs are tagged with a fluorescent and/or (bio)luminescent label such as GFP or nanoluc and the levels of fluorescence/bioluminescence are analyzed before and after storage/stress testing by spectrometer (SpectraMax) as well as using flow cytometry (e.g., CellStream®); and
iii) therapeutic functionality of EVs is tested before and after storage/stress testing using EVs loaded with a cargo, the effect of which can be observed in vivo. For instance, a splice switching cargo RNA may be added to the EVs. When those EVs are added to cells comprising a reporter that switches under the presence of the splice switching oligo, the effectiveness of the cargo loaded in the EVs is measurable. Alternatively, EVs expressing a decoy ligand on the surface, such as EVs genetically modified to display TNFalpha decoying receptors, may be tested in an NF-κB reporter cell model, which is genetically modified to express the NF-κB-luciferase reporter gene as a model for inflammation. The anti-inflammatory activity of the EVs comprising the TNFalpha decoying receptors can then be measured before and after storage/stress testing. In addition, albumin-display EVs carrying Cre can also be used to test the function of Cre using a Cre-Lox reporter in recipient cells before and after storage/stress testing.

### Example 9: Storage Stability of Lyophilised Albumin-Display EVs

Therapeutic EVs are commonly stored frozen at −20° C. or −80° C. The shelf-life of EVs stored at either of said temperatures is at most 1 year. This limits the accessibility of therapeutic EVs.

As shown from the examples above, the presence of albumin on the surface of EVs can increase the half-life, of EVs in vivo. This is attributable to the ability of the albumin protein according to the present invention.

The inventors thus anticipate that the presence of the albumin protein according to the present invention may equally be advantageous to improve the stability of EVs in storage.

It will be appreciated that albumin-display EVs according to the present invention can be stored in any suitable storage buffer for said EVs.

To investigate this hypothesis, a cross-section of albumin-display EVs according to the present invention were added to buffer. Buffer lacking an albumin-display EV and buffer containing non-engineered EVs were used as control. Furthermore, to test if addition of albumin to the storage buffer imparts any additional benefit a cross-section of albumin-display EVs according to the present invention were added to an albumin enriched buffer.

Stocks of EVs are typically stored in a freezer, when not in use. As such preparations containing EVs are subject to freeze thaw. Display of albumin protein on the surface of the EVs according to the present invention coat the outer surface of the EV providing a protective shield of albumin protein. Such a protective shield is believed to protect the EVs against damage caused by freeze thaw cycles and thus improve the stability in formulation in addition to improving half-life in vivo. This stability is anticipated to advantageously result in a proven increase shelf-life of the pharmaceutical composition of the EVs, making a more robust versatile product that will remain bioactive for longer during storage.

Albumin-display EVs are obtained from an EV-producing genetically engineered and immortalized cell line cultured in bioreactors in conditioned medium (CM). CM containing the EVs is harvested from the bioreactors. EVs are then isolated from the CM by centrifugation, to remove cells and cell debris, and thereafter filtrated to remove any larger particles. The filtered CM is then run through a hollow fiber filter using a TFF system and concentrated down after diafiltration. The EVs are then combined with a formulation buffer suitable for lyophilisation and comprising albumin. Protein expression was tested before (i.e., Pre-Lyo). This formulation is then freeze-dried and/or spray-dried using conventional methods.

This formulation is then either a) stored for a predetermined period (at different temperatures (e.g., −65° C., −20° C., 4° C.) for up to 30 weeks).

The lyophilised EV formulation was reconstituted at predetermined timepoints. Protein expression in the reconstituted formulation obtained post (Post-Lyo) lyophilization was tested. Pre-Lyo and Post-Lyo activity of the lyophilised EV formulation and controls across the chosen time-points was compared. Stability of the lyophilised EV formulation was then determined.

The quality and robustness of the EV population is then tested following the prolonged periods of time in storage at different temperatures as well as the repeated freeze thaw cycles. EV number, aggregates and other fragments are tested using nanoparticle tracking analysis (NTA). The quality of the EVs is measured using several techniques, for example those hereinbefore described in example 8.

### Example 10: Purification of Albumin-Display EVs

It is also predicted that the binding affinity of the albumin protein (ligand) according to the present invention for a corresponding receptor, for example FcRN, will allow the albumin-display EVs of the present invention to be purified using affinity chromatography. The steps of purification comprise: (i) contacting a medium comprising the albumin-display EVs with a chromatography matrix comprising FcRN (receptor), (ii) allowing the albumin protein displayed on the EVs of the present invention to adsorb to the FcRN, and (iii) eluting the albumin-display EVs by passing across the chromatography matrix a medium that releases the albumin-display EVs from the FcRN.

Albumin-display EVs are obtained from conditioned medium (CM) collected from genetically engineered EV-producing cell lines grown in a hollow-fibre bioreactor. The secreted EVs comprise albumin protein displayed on the surface. The CM obtained from the bioreactor is loaded onto a column connected to a chromatography system. The chromatography matrix comprises albumin bound to the surface. Flow rate settings for column equilibration, sample loading and column cleaning in place procedure are chosen according to the manufacturer's instructions. The medium comprising the albumin-display EVs is loaded onto the chromatography column and the albumin-display EVs bind to the matrix comprising albumin. An elution buffer is chosen to elute the albumin-display EVs from the column by altering the pH of the solution. The sample is then collected and stored at −80° C. for further downstream analysis, using flow cytometry, electron microscopy, and bioactivity assays.

Both the process of capturing the EVs and the process of releasing the EVs is repeated multiple times. The elution step comprises triggering the release of the albumin-display EVs from the FcRN by exposing the albumin-FcRN bond to a medium with a suitable pH. This is achieved by running the albumin-display EV-containing medium (i.e., the liquid phase) through a chromatography column comprising as stationary phase a chromatography matrix having attached to it a corresponding receptor suitable for binding the ligand (albumin protein), for example FcRN, letting the albumin protein of the EVs adsorb to the FcRN thereof present on the matrix, and then running a solution with a suitable pH through the chromatography column. The pH of the solution intended to trigger release of the EVs from the column may be below pH 8, below pH 7, or below pH 6.

The invention is not limited to the embodiments hereinbefore described which may be varied in both construction and detail.

SEQUENCE LISTING

Amino Acid Sequences of albumin protein
SEQ ID No 1: Human Serum Albumin (HSA)
MKWVTFISLLFLFSSAYSRGVFRRDAHKSEVAHRFKDLGEENFKALVLIAFA
QYLQQCPFEDHVKLVNEVTEFAKTCVADESAENCDKSLHTLFGDKLCTVAT
LRETYGEMADCCAKQEPERNECFLQHKDDNPNLPRLVRPEVDVMCTAFH
DNEETFLKKYLYEIARRHPYFYAPELLFFAKRYKAAFTECCQAADKAACLLP
KLDELRDEGKASSAKQGLKCASLQKFGERAFKAWAVARLSQRFPKAEFAE
VSKLVTDLTKVHTECCHGDLLECADDRADLAKYICENQDSISSKLKECCEKP
LLEKSHCIAEVENDEMPADLPSLAADFVGSKDVCKNYAEAKDVFLGMFLYE
YARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGS
KCCKHPEAKRMPCAEDCLSVFLNQLCVLHEKTPVSDRVTKCCTESLVNGR
PCFSALEVDETYVPKEFNAETFTFHADICTLSEKERQIKKQTALVELVKHKP
KATKEQLKAVMDDFAAFVEKCCKADDKETCFAEEGKKLVAASQAALGL
SEQ ID No 2: Human Albumin Domain III (DIII)
VEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESL
VNRRPCFSALEVDETYVPKEFNAETFTFHADICTLSEKERQIKKQTALVELV
KHKPKATKEQLKAVMDDFAAFVEKCCKADDKETCFAEEGKKLVAASQAAL
GL
Amino Acid Sequences of EV Proteins Used in Constructs
SEQ ID NO 3: LAMP2B
MVCFRLFPVPGSGLVLVCLVLGAVRSYALELNLTDSENATCLYAKWQMNFT
VRYETTNKTYKTVTISDHGTVTYNGSICGDDQNGPKIAVQFGPGFSWIANFT
KAASTYSIDSVSFSYNTGDNTTFPDAEDKGILTVDELLAIRIPLNDLFRCNSL
STLEKNDVVQHYWDVLVQAFVQNGTVSTNEFLCDKDKTSTVAPTIHTTVPS
PTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVF
SIANNNLSYWDAPLGSSYMCNKEQTVSVSGAFQINTFDLRVQPFNVTQGK
YSTAQECSLDDDTILIPIIVGAGLSGLIIVIVIAYVIGRRKSYAGYQTL
SEQ ID NO 4: LIMP2
MGRCCFYTAGTLSLLLLVTSVTLLVARVFQKAVDQSIEKKIVLRNGTEAFDS
WEKPPLPVYTQFYFFNVTNPEEILRGETPRVEEVGPYTYRELRNKANIQFG
DNGTTISAVSNKAYVFERDQSVGDPKIDLIRTLNIPVLTVIEWSQVHFLREIIE
AMLKAYQQKLFVTHTVDELLWGYKDEILSLIHVFRPDISPYFGLFYEKNGTN
DGDYVFLTGEDSYLNFTKIVEWNGKTSLDWWITDKCNMINGTDGDSFHPLI
TKDEVLYVFPSDFCRSVYITFSDYESVQGLPAFRYKVPAEILANTSDNAGFC
IPEGNCLGSGVLNVSICKNGAPIIMSFPHFYQADERFVSAIEGMHPNQEDHE
TFVDINPLTGIILKAAKRFQINIYVKKLDDFVETGDIRTMVFPVMYLNESVHID
KETASRLKSMINTTLIITNIPYIIMALGVFFGLVFTWLACKGQGSMDEGTADE
RAPLIRT
SEQ ID NO 5: TNFR
MGLSTVPDLLLPLVLLELLVGIYPSGVIGLVPHLGDREKRDSVCPQGKYIHPQ
NNSICCTKCHKGTYLYNDCPGPGQDTDCRECESGSFTASENHLRHCLSCS
KCRKEMGQVEISSCTVDRDTVCGCRKNQYRHYWSENLFQCFNCSLCLNG
TVHLSCQEKQNTVCTCHAGFFLRENECVSCSNCKKSLECTKLCLPQIENVK
GTEDSGTTVLLPLVIFFGLCLLSLLFIGLMYRYQRWKSKLYSIVCGKSTPEKE
GELEGTTTKPLAPNPSFSPTPGFTPTLGFSPVPSSTFTSSSTYTPGDCPNFA
APRREVAPPYQGADPILATALASDPIPNPLQKWEDSAHKPQSLDTDDPATLY
AVVENVPPLRWKEFVRRLGLSDHEIDRLELQNGRCLREAQYSMLATWRRR
TPRREATLELLGRVLRDMDLLGCLEDIEEALCGPAALPPAPSLLR
SEQ ID NO 6: TfR1
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTECERLA
GTESPVREEPGEDFPAARRLYWDDLKRKLSEKLDSTDFTGTIKLLNENSYV
PREAGSQKDENLALYVENQFREFKLSKVWRDQHFVKIQVKDSAQNSVIIVD
KNGRLVYLVENPGGYVAYSKAATVTGKLVHANFGTKKDFEDLYTPVNGSIVI
VRAGKITFAEKVANAESLNAIGVLIYMDQTKFPIVNAELSFFGHAHLGTGDPY
TPGFPSFNHTQFPPSRSSGLPNIPVQTISRAAAEKLFGNMEGDCPSDWKTD
STCRMVTSESKNVKLTVSNVLKEIKILNIFGVIKGFVEPDHYVVVGAQRDAW
GPGAAKSGVGTALLLKLAQMFSDMVLKDGFQPSRSIIFASWSAGDFGSVGA
TEWLEGYLSSLHLKAFTYINLDKAVLGTSNFKVSASPLLYTLIEKTMQNVKHP
VTGQFLYQDSNWASKVEKLTLDNAAFPFLAYSGIPAVSFCFCEDTDYPYLGT
TMDTYKELIERIPELNKVARAAAEVAGQFVIKLTHDVELNLDYERYNSQLLSF
VRDLNQYRADIKEMGLSLQWLYSARGDFFRATSRLTTDFGNAEKTDRFVM
KKLNDRVMRVEYHFLSPYVSPKESPFRHVFWGSGSHTLPALLENLKLRKQ
NNGAFNETLFRNQLALATWTIQGAANALSGDVWDIDNEF
Amino Acid Sequence of control Construct
SEQ ID No 7: hCD63-Nluc
MTGAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAAAIAGY
VFRDKVMSEFNNNFRQQMENYPKNNHTASILDRMQADFKCCGASGGSGS
GSFEANYTDWEKIPSMSKNRVPDSCCINVTVGCGINFNEKAIHKEGCVEKIG
GWLRKNVLVVAAAALGIAFVEVLGIVFACCLVKSIRSGYEVMACGSSGGSG
GGSGVFTLEDFVGDWRQTAGYNLDQVLEQGGVSSLFQNLGVSVTPIQRIV
LSGENGLKIDIHVIIPYEGLSGDQMGQIEKIFKVVYPVDDHHFKVILHYGTLVI
DGVTPNMIDYFGRPYEGIAVFDGKKITVTGTLWNGNKIIDERLINPDGSLLFR
VTINGVTGWRLCERILA
DNA Sequences of albumin protein
SEQ ID No 8: Human Serum Albumin (HSA)
ATGAAGTGGGTAACCTTTATTTCCCTTCTTTTTCTCTTTAGCTCGGCTTAT
TCCAGGGGTGTGTTTCGTCGAGATGCACACAAGAGTGAGGTTGCTCAT
CGGTTTAAAGATTTGGGAGAAGAAAATTTCAAAGCCTTGGTGTTGATTG
CCTTTGCTCAGTATCTTCAGCAGTGTCCATTTGAAGATCATGTAAAATTA
GTGAATGAAGTAACTGAATTTGCAAAAACtTGTGTTGCTGATGAGTCAGC
TGAAAATTGTGACAAATCACTTCATACCCTTTTcGGtGACAAATTATGCAC
AGTTGCAACTCTTCGTGAAACCTATGGTGAAATGGCTGACTGCTGTGCA
AAACAAGAACCTGAGAGAAATGAATGCTTCTTGCAACACAAAGATGACA
ACCCAAACCTCCCCCGATTGGTGAGACCAGAGGTTGATGTGATGTGCA
CTGCTTTTCATGACAATGAAGAGACATTTTTGAAAAAATACTTATATGAAA
TTGCCAGgAGACATCCaTAtTTcTATGCCCCGGAACTCCTTTTCTTTGCTA
AAAGGTATAAAGCTGCTTTTACAGAATGTTGCCAAGCTGCTGATAAAGCT
GCCTGCCTGTTGCCAAAGCTCGATGAACTTCGGGATGAAGGGAAGGCT
TCGTCTGCCAAACAGAGACTCAAGTGTGCCAGTCTCCAAAAATTTGGAG
AAAGAGCTTTCAAAGCATGGGCAGTAGCTCGCCTGAGCCAGAGATTTCC
CAAAGCTGAGTTTGCAGAAGTTTCCAAGTTAGTGACAGATCTTACCAAA
GTCCACACGGAATGCTGCCATGGAGATCTGCTTGAATGTGCTGATGACA
GGGCGGACCTTGCCAAGTATATCTGTGAAAATCAAGATTCGATCTCCAG
TAAACTGAAGGAATGCTGTGAAAAACCTCTGTTGGAAAAATCCCACTGC
ATTGCCGAAGTGGAAAATGATGAGATGCCTGCTGACTTGCCTTCATTAG
CTGCTGATTTTGTTGAAAGTAAGGATGTTTGCAAAAACTATGCTGAGGCA
AAGGATGTgTTCCTGGGCATGTTTTTGTATGAATATGCAAGAAGGCATCC
TGATTACTCTGTCGTGCTGCTGCTGAGACTTGCCAAGACATATGAAACC
ACTCTAGAGAAGTGCTGTGCCGCTGCAGATCCTCATGAATGCTATGCCA
AAGTGTTCGATGAATTTAAACCTCTTGTGGAAGAGCCTCAGAATTTAATC
AAACAAAATTGTGAGCTTTTTGAGCAGCTTGGAGAGTACAAATTCCAGAA
TGCGCTATTAGTTCGTTACACCAAGAAAGTACCCCAAGTGTCAACTCCA
ACTCTTGTAGAGGTCTCAAGAAACCTAGGAAAAGTGGGatcCAAATGTTG
TAAACATCCTGAAGCAAAAAGAATGCCCTGTGCAGAgGACTATCTATCC
GTGGTCCTGAACCAGTTATGTGTGTTGCATGAGAAAACGCCAGTAAGTG
ACAGAGTCACCAAATGCTGCACAGAATCCTTGGTGAACAGGCGACCAT
GCTTTTCAGCTCTGGAAGTCGATGAAACATACGTTCCCAAAGAGTTTAAT
GCTGAAACATTCACCTTCCATGCAGATATATGCACACTTTCTGAGAAGG
AGAGACAAATCAAGAAACAAACTGCACTTGTTGAGCTCGTGAAACACAA
GCCCAAGGCAACAAAAGAGCAACTGAAAGCTGTTATGGATGATTTCGCA
GCTTTTGTAGAGAAGTGCTGCAAGGCTGACGATAAGGAGACCTGCTTTG
CCGAGGAGGGTAAAAAACTTGTTGCTGCAAGTCAAGCTGCCTTAGGCTT
A
SEQ ID NO 9: DIII of human serum albumin
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAG
AAAGTACCCCAAGTGTCAACTCCAACTCTTGTAGAGGTCTCAAGAAACC
TAGGAAAAGTGGGGTCCAAATGTTGTAAACATCCTGAAGCAAAAAGAAT
GCCCTGTGCAGAgGACTATCTATCCGTGGTCCTGAACCAGTTATGTGTG
TTGCATGAGAAAACGCCAGTAAGTGACAGAGTCACCAAATGCTGCACAG
AATCCTTGGTGAACAGGCGACCATGCTTTTCAGCTCTGGAAGTCGATGA
AACATACGTTCCCAAAGAGTTTAATGCTGAAACATTCACCTTCCATGCAG
ATATATGCACACTTTCTGAGAAGGAGAGACAAATCAAGAAACAAACTGC
ACTTGTTGAGCTCGTGAAACACAAGCCCAAGGCAACAAAAGAGCAACTG
AAAGCTGTTATGGATGATTTCGCAGCTTTTGTAGAGAAGTGCTGCAAGG
CTGACGATAAGGAGACCTGCTTTGCCGAGGAGGGTAAAAAACTTGTTGC
TGCAAGTCAAGCTGCCTTAGGCTTA
DNA Sequences of EV Proteins Used in Constructs
SEQ ID NO 10: Lamp2B
ATG GTT TGC TTC AGG TTG TTT CCT GTT CCA GGT AGC GGC TTG GTT
CTC GTC TGT CTT GTA CTC GGC GCC GTA AGG AGC TAC GCT TTG
GAG CTG AAT CTT ACT GAC TCT GAA AAC GCC ACA TGT TTG TAT GCC
AAG TGG CAG ATG AAT TTC ACC GTC AGG TAC GAG ACG ACT AAC
AAG ACC TAC AAG ACG GTC ACT ATC AGC GAC CAC GGC ACT GTG
ACA TAT AAC GGG TCC ATC TGC GGG GAC GAT CAG AAC GGC CCA
AAG ATT GCT GTT CAA TTT GGC CCC GGC TTC AGT TGG ATT GCC AAC
TTT ACC AAA GCT GCG AGC ACT TAC TCT ATT GAT TCA GTC AGT TTC
TCA TAC AAC ACC GGC GAT AAC ACG ACA TTT CCG GAC GCA GAA
GAT AAG GGG ATT CTG ACT GTT GAT GAG CTG CTC GCT ATT CGC
ATC CCC CTT AAT GAC CTT TTT CGG TGC AAC AGT CTT TCC ACA CTC
GAG AAG AAT GAC GTC GTT CAG CAC TAC TGG GAC GTT CTG GTC
CAA GCA TTT GTG CAA AAT GGC ACG GTA AGC ACT AAC GAG TTT CTG
TGC GAC AAG GAC AAG ACT AGC ACT GTC GCC CCG ACT ATC CAT
ACA ACT GTC CCA AGC CCT ACG ACC ACT CCG ACT CCC AAG GAG
AAA CCA GAG GCT GGG ACG TAT AGC GTC AAC AAT GGG AAT GAT
ACA TGC TTG CTG GCC ACA ATG GGA CTC CAA CTG AAT ATC ACC
CAG GAC AAG GTG GCC TCA GTT ATA AAT ATA AAT CCT AAT ACA ACA
CAT AGT ACT GGA AGT TGT CGC TCC CAT ACC GCC TTG TTG AGG
TTG AAC AGC TCA ACG ATA AAG TAT CTC GAT TTC GTC TTT GCC GTA
AAG AAT GAA AAC CGA TTC TAT TTG AAG GAG GTC AAT ATA AGT ATG
TAC CTG GTA AAC GGA TCA GTA TTT AGT ATT GCT AAT AAC AAT CTC
TCT TAC TGG GAC GCT CCT CTC GGA AGT AGT TAT ATG TGT AAC AAG
GAA CAG ACC GTC TCT GTA TCC GGA GCG TTC CAG ATC AAT ACT TTC
GAC CTG CGC GTA CAA CCT TTC AAT GTT ACG CAA GGG AAG TAC
TCC ACA GCC CAG GAG TGT TCT CTT GAC GAT GAC ACC ATT TTG ATC
CCG ATC ATT GTT GGA GCA GGG TTG AGT GGG CTG ATT ATT GTT ATT
GTA ATC GCT TAT GTT ATA GGA CGA AGG AAG AGT TAC GCC GGC
TAT CAG ACG CTT
SEQ ID NO 11: LIMP2
ATG GGG CGA TGT TGC TTT TAC ACG GCG GGT ACC CTG AGC TTG
TTG CTC CTC GTG ACA AGT GTC ACT CTT TTG GTA GCT AGG GTG TTT
CAG AAG GCC GTA GAC CAA TCT ATA GAA AAA AAG ATT GTT CTT AGA
AAT GGT ACC GAA GCG TTC GAT TCT TGG GAG AAA CCA CCA CTC
CCG GTA TAT ACC CAG TTC TAC TTC TTC AAT GTA ACC AAC CCA GAG
GAG ATT CTT CGA GGT GAG ACC CCT CGC GTG GAA GAA GTA GGA
CCA TAC ACC TAT CGG GAA CTT CGC AAC AAA GCT AAT ATT CAA TTC
GGG GAT AAT GGC ACC ACG ATA AGC GCA GTA AGC AAT AAG GCG
TAT GTG TTT GAA CGA GAT CAG AGT GTG GGT GAT CCT AAG ATC GAC
CTC ATC CGC ACT CTC AAT ATC CCC GTA TTG ACA GTT ATT GAG TGG
TCT CAA GTT CAT TTT CTC CGA GAG ATT ATA GAA GCG ATG CTT AAG
GCG TAC CAA CAA AAG TTG TTC GTC ACC CAT ACT GTG GAT GAA CTC
CTG TGG GGA TAT AAG GAT GAA ATT TTG AGT TTG ATC CAC GTA TTT
AGA CCA GAC ATT TCA CCC TAT TTT GGC TTG TTC TAT GAG AAA AAT
GGA ACG AAC GAC GGG GAT TAC GTA TTC CTC ACC GGT GAG GAC
TCA TAC CTG AAC TTC ACT AAA ATA GTC GAG TGG AAC GGA AAG ACA
TCT CTG GAC TGG TGG ATT ACT GAT AAG TGC AAT ATG ATA AAC GGC
ACT GAC GGG GAT TCT TTC CAT CCC CTG ATC ACG AAA GAT GAG
GTA CTC TAC GTA TTC CCG AGC GAT TTC TGT CGG AGC GTC TAC ATC
ACA TTT TCC GAT TAC GAG AGC GTC CAA GGT CTG CCA GCC TTC
CGG TAT AAA GTA CCA GCT GAA ATA CTT GCT AAC ACC AGT GAC AAT
GCC GGA TTT TGT ATA CCA GAG GGT AAT TGT TTG GGG AGT GGA
GTG CTC AAC GTG TCA ATT TGC AAG AAC GGG GCA CCT ATA ATC ATG
TCT TTT CCA CAC TTC TAT CAA GCT GAT GAG CGG TTC GTA AGC GCC
ATA GAA GGT ATG CAT CCG AAT CAA GAG GAC CAT GAA ACC TTC
GTG GAT ATA AAC CCG CTC ACT GGC ATA ATT TTG AAG GCC GCA
AAG AGA TTC CAG ATT AAT ATA TAC GTT AAG AAG CTT GAT GAT TTC
GTT GAA ACC GGA GAC ATT CGG ACT ATG GTA TTT CCT GTA ATG TAC
TTG AAC GAA TCC GTA CAC ATC GAT AAG GAG ACA GCG AGT CGC
CTT AAA AGT ATG ATA AAT ACG ACT CTT ATA ATC ACA AAC ATC CCT
TAT ATT ATA ATG GCC TTG GGT GTG TTC TTT GGA TTG GTG TTT ACA
TGG TTG GCA TGT AAG GGC CAA GGG TCC ATG GAT GAG GGG ACG
GCT GAT GAA AGA GCC CCT TTG ATT CGC ACT
SEQ ID NO 12: TNFR
ATG GGC CTT AGT ACG GTT CCG GAT TTG TTG CTC CCA CTC GTC TTG
CTG GAG CTT CTT GTC GGC ATA TAT CCC AGC GGA GTC ATC GGC
TTG GTG CCG CAT CTT GGC GAC CGA GAG AAA CGG GAT AGT GTG
TGC CCG CAA GGG AAA TAT ATC CAT CCA CAG AAC AAT TCC ATT TGT
TGT ACA AAG TGT CAC AAG GGA ACA TAT TTG TAC AAC GAC TGT CCC
GGA CCT GGA CAA GAC ACC GAC TGT CGG GAG TGT GAA TCC GGG
TCT TTT ACT GCG AGC GAA AAC CAC CTC AGG CAT TGT CTC AGT TGC
TCC AAA TGC CGG AAA GAA ATG GGG CAG GTG GAA ATT TCA TCA
TGC ACT GTG GAC CGC GAC ACT GTT TGC GGT TGC CGG AAG AAC
CAA TAT CGG CAT TAC TGG AGC GAA AAT CTC TTT CAA TGT TTC AAC
TGT TCA CTG TGT CTC AAC GGT ACT GTA CAT CTC TCT TGT CAG GAG
AAG CAG AAT ACT GTT TGT ACT TGT CAC GCC GGG TTT TTT TTG AGG
GAA AAT GAG TGC GTT AGC TGC AGT AAT TGT AAG AAG TCA CTC GAA
TGC ACA AAG CTG TGC TTG CCC CAG ATA GAA AAC GTA AAG GGA
ACG GAA GAT AGT GGG ACG ACG GTG CTC CTC CCT CTG GTG ATA
TTT TTC GGA CTT TGC CTC CTG AGC CTG CTT TTT ATA GGG CTC ATG
TAT CGC TAC CAG CGG TGG AAG TCC AAG CTG TAC AGT ATA GTG
TGT GGT AAG TCT ACA CCT GAA AAG GAG GGG GAA CTC GAA GGA
ACC ACA ACG AAG CCT TTG GCA CCG AAC CCG TCA TTT TCA CCC
ACG CCG GGT TTC ACC CCG ACA CTG GGA TTC TCC CCC GTA CCT
TCT TCA ACA TTC ACA TCA AGC AGT ACA TAC ACT CCT GGG GAC TGT
CCC AAT TTC GCA GCT CCT AGG CGC GAG GTG GCA CCC CCC TAC
CAA GGT GCT GAT CCT ATT CTC GCA ACT GCA CTG GCG AGC GAT
CCA ATA CCG AAC CCC CTG CAG AAA TGG GAG GAT TCA GCC CAC
AAG CCG CAA TCT CTG GAT ACT GAC GAC CCA GCC ACT CTC TAT
GCC GTA GTT GAG AAC GTA CCG CCT CTC AGG TGG AAA GAA TTC
GTG CGA AGG TTG GGC CTG TCT GAT CAT GAA ATA GAC CGA CTC
GAA CTC CAG AAT GGT CGC TGC CTT CGA GAG GCT CAG TAC TCA
ATG CTC GCT ACC TGG CGC AGG AGG ACA CCA CGG AGG GAG GCT
ACC CTT GAA CTG CTT GGG CGC GTA CTG CGG GAC ATG GAT CTG
CTT GGC TGC CTT GAG GAT ATA GAG GAG GCA CTG TGC GGC CCT
GCC GCA CTG CCA CCA GCC CCG AGT TTG CTG CGG
SEQ ID NO 13: TfR1
ATG ATG GAC CAG GCG CGA TCC GCC TTT TCA AAC TTG TTC GGA
GGA GAG CCT CTG AGT TAT ACA AGG TTC AGT CTG GCC CGA CAA
GTG GAC GGA GAC AAT TCA CAC GTC GAG ATG AAA CTT GCT GTT
GAT GAG GAA GAG AAT GCG GAC AAT AAT ACG AAA GCA AAC GTG
ACA AAA CCC AAA AGA TGT AGC GGG TCC ATC TGC TAC GGA ACG
ATC GCC GTC ATC GTC TTT TTT CTT ATA GGG TTC ATG ATC GGA TAT
CTC GGG TAT TGT AAA GGC GTC GAG CCG AAA ACT GAA TGC GAA
CGA CTC GCT GGA ACA GAG TCT CCT GTT AGA GAG GAG CCT GGG
GAG GAT TTC CCT GCA GCG CGA CGG CTG TAT TGG GAT GAT TTG
AAG AGG AAA CTG TCT GAA AAG CTG GAT TCC ACG GAC TTC ACG
GGC ACG ATC AAA TTG CTT AAT GAG AAT TCC TAT GTC CCG CGG
GAG GCT GGT TCA CAA AAA GAC GAG AAC CTG GCG TTG TAT GTC
GAA AAT CAA TTC CGC GAA TTC AAG TTG AGT AAG GTA TGG CGA GAT
CAA CAC TTC GTA AAA ATA CAA GTC AAG GAT TCC GCG CAA AAC AGT
GTA ATC ATC GTT GAT AAA AAT GGA CGG CTT GTG TAT TTG GTG GAA
AAT CCG GGC GGG TAT GTG GCA TAT AGC AAG GCC GCA ACT GTA
ACA GGC AAG CTT GTA CAC GCC AAT TTC GGC ACC AAG AAA GAT TTT
GAG GAC CTT TAC ACT CCC GTC AAT GGA AGT ATA GTG ATT GTG AGA
GCG GGC AAG ATT ACC TTT GCG GAA AAA GTA GCT AAT GCA GAG
TCC TTG AAC GCG ATA GGG GTC CTC ATC TAT ATG GAT CAG ACG
AAG TTC CCG ATT GTA AAC GCC GAG CTG AGC TTC TTC GGA CAC
GCG CAC CTG GGA ACG GGG GAT CCT TAC ACA CCG GGA TTT CCA
AGT TTC AAT CAC ACT CAG TTT CCG CCG AGT CGA TCC AGT GGA CTC
CCT AAC ATT CCT GTC CAG ACG ATA TCC CGA GCA GCG GCA GAA
AAA CTT TTC GGG AAT ATG GAA GGC GAC TGC CCT AGC GAC TGG
AAG ACT GAT TCA ACT TGT AGA ATG GTG ACC TCT GAG AGC AAG AAT
GTC AAG CTG ACC GTC TCC AAT GTC CTT AAG GAG ATT AAG ATT CTG
AAT ATC TTC GGA GTC ATA AAG GGG TTC GTA GAG CCG GAT CAC
TAC GTC GTG GTT GGA GCA CAG AGG GAC GCG TGG GGA CCC GGT
GCT GCA AAG AGT GGA GTG GGC ACA GCG TTG CTT CTT AAG CTC
GCG CAG ATG TTC TCC GAC ATG GTG CTT AAA GAT GGG TTT CAA
CCC TCT AGA AGC ATT ATT TTC GCA TCT TGG TCT GCA GGC GAT TTC
GGG TCA GTT GGG GCG ACA GAA TGG CTT GAG GGC TAC CTC AGT
TCT CTG CAT CTC AAG GCC TTC ACT TAT ATT AAC CTG GAC AAA GCT
GTA TTG GGT ACG TCA AAC TTC AAG GTG AGT GCT TCC CCA CTC CTG
TAC ACA CTG ATA GAA AAG ACT ATG CAA AAT GTA AAG CAC CCC GTG
ACG GGC CAA TTC CTT TAC CAG GAC TCA AAT TGG GCC TCA AAG
GTG GAA AAA CTC ACT CTC GAC AAC GCA GCG TTC CCC TTC CTG
GCC TAT AGC GGC ATC CCG GCG GTG TCT TTC TGC TTT TGT GAA
GAC ACC GAC TAC CCC TAT TTG GGT ACC ACG ATG GAT ACC TAT AAA
GAG CTG ATA GAG AGG ATC CCG GAA CTC AAT AAA GTT GCC AGG
GCA GCG GCC GAG GTG GCA GGA CAG TTC GTG ATT AAA CTC ACT
CAC GAT GTC GAA CTG AAT CTC GAT TAT GAA CGG TAT AAC TCT CAA
CTC CTG TCC TTT GTC CGC GAT CTG AAC CAA TAC AGA GCG GAC ATT
AAA GAA ATG GGA CTG TCA CTC CAG TGG CTC TAC TCT GCT CGC
GGT GAC TTC TTT AGG GCA ACG TCC AGA CTG ACG ACT GAT TTC
GGT AAT GCT GAG AAA ACC GAT CGC TTC GTG ATG AAG AAA CTG
AAT GAC AGG GTG ATG CGG GTC GAA TAT CAC TTC CTC TCA CCT TAT
GTT TCT CCA AAG GAG TCC CCT TTC CGG CAT GTC TTT TGG GGC
AGC GGA AGC CAT ACG CTG CCC GCA TTG CTT GAA AAT CTG AAA
CTG AGA AAG CAG AAT AAT GGA GCA TTT AAT GAA ACG TTG TTT CGC
AAC CAG CTT GCC CTC GCG ACG TGG ACT ATA CAG GGT GCG GCG
AAT GCT TTG AGC GGA GAT GTT TGG GAT ATA GAC AAT GAA TTT
DNA Sequence of Control Construct and Exemplary Reporter
SEQ ID NO 14: hCD63-Nluc
ATGACCGGTGCTGTGGAAGGCGGCATGAAATGCGTGAAGTTTCTGCTG
TATGTTCTGCTCTTGGCATTTTGTGCGTGTGCCGTTGGCCTTATTGCAGT
CGGTGTAGGTGCTCAGTTGGTACTCAGTCAAACAATTATTCAGGGCGCA
ACACCCGGCTCTTTGTTGCCGGTGGTGATTATCGCTGTTGGTGTATTCC
TTTTTTTGGTGGCCTTTGTTGGTTGCTGCGGGGCGTGCAAGGAGAATTA
CTGTTTGATGATTACCTTTGCCATATTTCTGTCATTGATTATGCTGGTAG
AGGTTGCAGCGGCGATTGCAGGGTACGTGTTTAGAGATAAAGTGATGT
CAGAGTTTAACAACAATTTTAGGCAGCAGATGGAAAATTACCCGAAAAA
CAACCACACAGCTTCAATCCTGGACAGAATGCAAGCGGACTTTAAATGT
TGCGGTGCCTCCGGAGGTAGCGGCAGCGGTAGCTTCGAAGCGAACTAT
ACTGATTGGGAGAAAATTCCGAGTATGTCTAAGAATCGAGTACCAGATA
GTTGCTGTATAAATGTGACGGTCGGATGCGGAATAAACTTCAATGAAAA
GGCTATTCACAAAGAGGGATGTGTCGAGAAAATTGGTGGATGGCTGAG
GAAGAATGTACTGGTGGTTGCTGCCGCCGCCCTGGGCATCGCATTCGT
AGAGGTCCTCGGAATCGTATTTGCGTGTTGTTTGGTTAAGTCTATACGG
AGCGGGTATGAAGTCATGGCATGCGGCAGTTCTGGAGGCTCAGGCGG
CGGATCTGGCGTCTTCACATTGGAAGACTTCGTTGGGGATTGGAGGCA
GACCGCCGGGTATAACTTGGATCAGGTACTCGAACAAGGCGGAGTATC
TTCCCTGTTCCAAAATCTGGGGGTTAGCGTCACGCCCATTCAACGCATT
GTGCTCTCCGGTGAGAATGGACTGAAGATCGATATACACGTAATTATCC
CCTATGAGGGGCTGTCTGGTGATCAGATGGGGCAGATAGAGAAAATCT
TTAAGGTAGTTTACCCCGTGGATGATCACCACTTCAAAGTAATCCTCCAT
TATGGTACACTCGTGATTGACGGGGTCACACCAAACATGATCGATTACT
TCGGCAGACCGTATGAGGGCATAGCAGTATTCGACGGAAAGAAGATAA
CAGTCACGGGGACATTGTGGAATGGAAACAAAATAATTGATGAACGCCT
CATCAACCCGGACGGAAGTCTTCTTTTTCGCGTCACAATCAACGGCGTG
ACGGGGTGGCGGCTCTGTGAAAGGATCTTGGCGTAG
SEQ ID NO 15: Nluc
GTCTTCACATTGGAAGACTTCGTTGGGGATTGGAGGCAGACCGCCGGG
TATAACTTGGATCAGGTACTCGAACAAGGCGGAGTATCTTCCCTGTTCC
AAAATCTGGGGGTTAGCGTCACGCCCATTCAACGCATTGTGCTCTCCGG
TGAGAATGGACTGAAGATCGATATACACGTAATTATCCCCTATGAGGGG
CTGTCTGGTGATCAGATGGGGCAGATAGAGAAAATCTTTAAGGTAGTTT
ACCCCGTGGATGATCACCACTTCAAAGTAATCCTCCATTATGGTACACT
CGTGATTGACGGGGTCACACCAAACATGATCGATTACTTCGGCAGACC
GTATGAGGGCATAGCAGTATTCGACGGAAAGAAGATAACAGTCACGGG
GACATTGTGGAATGGAAACAAAATAATTGATGAACGCCTCATCAACCCG
GACGGAAGTCTTCTTTTTCGCGTCACAATCAACGGCGTGACGGGGTGG
CGGCTCTGTGAAAGGATCTTGGCG

1. An Extracellular Vesicle (EV) modified to comprise an albumin protein present on the surface of the EV.

2. The EV according to claim 1, wherein the albumin protein forms part of a fusion protein with an EV protein.

3. The EV according to claim 2, wherein the EV protein is selected from an EV transmembrane protein or EV membrane associated protein.

4. The EV according to claim 3, wherein the transmembrane protein is a single-pass transmembrane protein or a multi-pass transmembrane protein.

5. The EV according to claim 3, wherein the transmembrane protein is a tetraspanin.

6. The EV according to claim 2, wherein the EV protein is selected from one or more of CD44, CD47, CD55, LAMP2B, TFNR, TfR1, CD63, CD81, CD82, CD9, LIMP2, ICAMs, ARRDC1, TSPAN2, TSPAN3 and derivatives, domains, variants, mutants, or regions thereof.

7. The EV according to claim 2, wherein the albumin protein is fused to the EV protein at the N-terminus, C-terminus, at least a portion of a transmembrane domain, or at least a portion of extravesicular loop of the EV protein.

8. (canceled)

9. The EV according to claim 1, wherein the EV comprises a plurality of albumin proteins.

10. The EV according to claim 9, wherein at least two of the albumin proteins of the plurality of albumin proteins are present in the same fusion protein as each other.

11. The EV according to claim 9, wherein the albumin proteins are fused to the same extracellular loop of the same fusion protein as one another.

12. The modified EV according to claim 9, wherein the albumins or modified albumins are fused to a different extracellular loop of the same fusion protein from one another.

13. The EV according to claim 9, wherein at least two of the albumin proteins of the plurality of albumin proteins are present in a different fusion protein from each other.

14. The EV according to claim 1, wherein the EV comprises a cargo.

15. The EV according to claim 14, wherein the cargo is a therapeutic cargo selected from one or more of a peptide, a protein, a nucleic acid, a virus, a viral genome, an antigen, a small molecule or a biologic.

16. The EV according to claim 14, wherein the cargo is present on the inside of the EV, on the outside of the EV and/or in the membrane of the EV.

17. The EV according to claim 14, wherein the therapeutic cargo forms part of the albumin fusion protein.

18. The EV according to claim 1, wherein the EV further comprises a targeting moiety.

19. The EV according to claim 17, wherein the targeting moiety forms part of the albumin fusion protein.

20. The EV according to claim 1, wherein the EV is an exosome.

21. A method for producing the EV according to claim 1, wherein the method comprises the steps of:
(i) introducing into an EV-producing cell at least one polynucleotide construct encoding an albumin-EV protein fusion construct; and
(ii) expressing said construct in the EV-producing cell, thereby generating an EV comprising one or more of an albumin protein present on the surface of the EV.

22. A pharmaceutical composition comprising at least one EV according to claim 1 and one or more pharmaceutically acceptable excipient, diluent, vehicle, solvent or carrier.

23. (canceled)

24. A method of treating a disease or condition in a subject in need thereof comprising, administering to the subject a therapeutically effective amount of the EV according to claim 1.
